(19) United States
(12) Patent Application Publication
(10) Pub. No.: US 2024/0239907 A1
(43) Pub. Date: Jul. 18, 2024

(54) C-X-C Motif Chemokine Receptor 6 (CXCR6) Binding Molecules, and Methods of Using the Same

(71) Applicant: Edelweiss Immune Inc, Cambridge, MA (US)

(72) Inventors: Lifei Hou, Dover, MA (US); Eileen Remold-O'Donnell, Brookline, MA (US)

(21) Appl. No.: 18/563,720

(22) PCT Filed: May 25, 2022
(86) PCT No.: PCT/US2022/072573

§ 371 Date: Nov. 22, 2023

Related U.S. Application Data
(60) Provisional application No. 63/193,060, filed on May 25, 2021.

(51) Int. Cl. C07K 16/28; A61K 31/137; A61K 31/506; A61K 31/519; A61K 31/5377; A61K 31/573; A61K 31/675; A61K 39/395; A61K 47/68; A61K 49/00

(52) U.S. Cl. CPC C07K 16/2866; A61K 31/137; A61K 31/506; A61K 31/519; A61K 31/5377; A61K 31/573; A61K 31/675; A61K 39/3955; A61K 47/6849; A61K 49/0002; C07K 2317/622; C07K 2319/30

(57) ABSTRACT

The invention provides, in various embodiments, polypeptides that specifically bind to C-X-C Motif Chemokine Receptor 6 (CXCR6) (e.g., human CXCR6). The invention also provides, in various embodiments, fusion proteins comprising one or more of the polypeptides, polynucleotides encoding the polypeptides, vectors and host cells suitable for expressing the polypeptides, and compositions and methods for treating, diagnosing, and/or prognosing diseases or disorders (e.g., diseases of the immune system or cancer).

### RELATED APPLICATION(S)

This application claims the benefit of U.S. Provisional Application No. 63/193,060, filed on May 25, 2021. The entire teachings of the above application(s) are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

File name: 57471000001_SequenceListing.txt; created May 24, 2022, 164,000 Bytes in size.

### BACKGROUND

T lymphocytes, including both CD4 and CD8 T cells, play a pivotal role in immune disorders and in tumors. The activation of T cells includes two essential signals. First, the T cell receptor (TCR) binds to the antigen-loaded MHC molecule on the antigen-presenting cells (APCs). Second, CD28 and CD40L on T cells interact with B7 and CD40 on APCs, respectively (co-stimulatory signaling). Appropriate T cell activation signaling results in cytokine production and proliferation as well as active killing of tumor cells. To restrict overzealous activation, T cells are induced to express inhibitory molecules, such as cytotoxic T-lymphocyte-associated protein 4 (CTLA-4) and programmed cell death protein 1 (PD-1), to transduce co-inhibitory signals by binding to B7 expressed by APCs and PD-L1, respectively. PD-L1 is expressed by tumors and APCs.

Pathogenic T cells, a subset of highly differentiated hyper-activated T cells, are the driving force for inflammation and tissue damage in immune disorders such as autoimmune diseases, allograft rejection, acute or chronic graft versus host disease, bone marrow transplantation, T cell-driven cytokine storm, allergic disease and acute viral infection. Currently available immunosuppressive drugs include steroids, cyclosporine A, cytotoxic agents (e.g., cyclophosphamide and methotrexate), natalizumab (blocking leukocyte adhesion), and fingolimod (preventing lymphocyte egress from lymphoid tissues), etc. While these drugs can ameliorate disease symptoms, because they also impair the normal immune system and immune defense, they often cause severe side effects and intolerable risks of infection. Thus, new therapeutics that selectively target pathogenic T cells, are needed for treating immune disorders.

In the contrasting scenario of tumor pathology, T cells infiltrate into the tumor to eliminate the tumor cells. However, due to the immunosuppressive microenvironment within the tumor, the infiltrated T cells are often altered toward functional exhaustion with impaired cytokine production, cytotoxicity, proliferation and survival. Currently available antibody therapeutics block immune checkpoint inhibitors such as cytotoxic T-lymphocyte-associated protein 4 (CTLA4), programmed cell death protein 1 (PD1), and programmed death-ligand 1 (PD-L1). Because those drugs non-selectively activate the immune system, they have resulted in isolated cases of serious autoimmune symptoms. Thus, new therapeutics that selectively target tumor-infiltrating T cells, are needed for treating cancer.

### SUMMARY

The invention disclosed herein is based, at least in part, on the discovery that C-X-C motif chemokine receptor 6 (CXCR6) is an exquisite marker of pathogenic T cells as well as a specific marker of immune-competent T cells infiltrated in a tumor.

In one aspect, the invention provides a polypeptide that specifically binds a CXCR6, the polypeptide comprising heavy chain complementarity determining regions HCDR1, HCDR2 and HCDR3 that are at least about 90% identical to the HCDR1, HCDR2 and HCDR3, respectively, of at least one immunoglobulin heavy chain variable region (V_{H}) set forth in SEQ ID NOs: 11-46. In some embodiments, the polypeptide binds human and cyno monkey CXCR6 (e.g., the polypeptide comprises heavy chain complementarity determining regions HCDR1, HCDR2 and HCDR3 that are at least about 90% identical to the HCDR1, HCDR2 and HCDR3, respectively, of at least one immunoglobulin heavy chain variable region (V_{H}) set forth in at least one immunoglobulin heavy chain variable region (V_{H}) set forth in SEQ ID NOS: 11-17). In some embodiments, the polypeptide binds human CXCR6 but not cyno monkey CXCR6 (e.g., the polypeptide comprises heavy chain complementarity determining regions HCDR1, HCDR2 and HCDR3 that are at least about 90% identical to the HCDR1, HCDR2 and HCDR3, respectively, of at least one immunoglobulin heavy chain variable region (V_{H}) set forth in at least one immunoglobulin heavy chain variable region (V_{H}) set forth in SEQ ID NOS: 18-46).

In some embodiments, the polypeptide further comprises light chain complementarity determining regions LCDR1, LCDR2 and LCDR3 that are at least 90% identical to the LCDR1, LCDR2 and LCDR3, respectively, of at least one immunoglobulin light chain variable region (V_{L}) set forth in SEQ ID NOs:47-82.

In another aspect, the invention provides a fusion protein comprising one or more of the polypeptides described herein.

In another aspect, the invention provides one or more polynucleotides encoding one or more of the polypeptides or fusion proteins described herein.

In another aspect, the invention provides an expression vector comprising one or more of the polynucleotides described herein.

In another aspect, the invention provides a host cell (e.g., an expression host cell) comprising one or more of the polynucleotides or expression vectors described herein.

In another aspect, the invention provides a composition comprising one or more of the polypeptides, fusion proteins, polynucleotides, expression vectors or host cells described herein. In some embodiments, the composition is a pharmaceutical composition. In some embodiments, the composition comprises one or more of the polypeptides or fusion proteins described herein.

In another aspect, the invention provides a method of modulating a CXCR6-positive leukocyte, comprising contacting the CXCR6-positive leukocyte with one or more of the polypeptides, fusion proteins or compositions described herein.

In another aspect, the invention provides a method of blocking chemotaxis of a CXCR6-positive leukocyte, comprising contacting the CXCR6-positive leukocyte with one or more of the polypeptides, fusion proteins or compositions described herein.

In another aspect, the invention provides a method of inactivating a CXCR6-positive leukocyte, comprising contacting the CXCR6-positive leukocyte with one or more of the polypeptides, fusion proteins or compositions described herein.

In another aspect, the invention provides a method of killing a CXCR6-positive leukocyte, comprising contacting the CXCR6-positive leukocyte with one or more of the polypeptides, fusion proteins or compositions described herein.

In another aspect, the invention provides a method of activating a CXCR6-positive leukocyte, comprising contacting the CXCR6-positive leukocyte with one or more of the polypeptides, fusion proteins or compositions described herein.

In another aspect, the invention provides a method of maintaining the survival of a CXCR6-positive leukocyte, comprising contacting the CXCR6-positive leukocyte with one or more of the polypeptides, fusion proteins or compositions described herein.

In another aspect, the invention provides a method of depleting of CXCR6-positive leukocytes in a subject in need thereof, comprising administering to the subject an effective amount of one or more of the compositions (e.g., pharmaceutical compositions) described herein.

In another aspect, the invention provides a method of treating or preventing a disease in a subject in need thereof, comprising administering to the subject an effective amount with one or more of the compositions (e.g., pharmaceutical compositions) described herein.

In another aspect, the invention provides a method of activating CXCR6-positive cells in a subject in need thereof, comprising administering to the subject an effective amount of one or more of the compositions (e.g., pharmaceutical compositions) described herein.

In another aspect, the invention provides a method of maintaining the survival of CXCR6-positive cells in a subject in need thereof, comprising administering to the subject an effective amount of one or more of the compositions (e.g., pharmaceutical compositions) described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 depicts a scheme of acute Graft versus Host Disease (aGvHD) treatment via CXCR6 monoclonal antibody (mAb)-mediated depletion of CXCR6-positive pathogenic T cells.

FIG. 2 depicts a scheme of cancer therapy via CXCR6 mAb-mediated expansion of CXCR6-positive immune-competent T cells.

FIGS. 3A-3B show that CXCR6-positive CD4 T cells are the major population in collagen-induced arthritis that produces pro-inflammatory cytokines interleukin (IL)-17 and interferon gamma (IFNγ). FIG. 3A shows cytokine profiles in CXCR6-negative or CXCR6-positive CD4 T cells of the spleen. FIG. 3B shows cytokine profiles in CXCR6-negative or CXCR6-positive CD4 T cells of the lymph node. Each symbol represents an individual mouse. **: p<0.01; ***: p<0.001. Black circle: CXCR6-negative CD4 T cell. Black triangle: CXCR6-positive CD4 T cell.

FIGS. 4A-4C show that CXCR6-positive T cells accumulate in aGvHD (C57BL/6 into BALB/C). FIG. 4A shows decreased body weight in aGvHD diseased mice. FIG. 4B shows early death in aGvHD diseased mice. FIG. 4C compares percent of CXCR6-positive CD4 and CD8 T cells in the lymph node and spleen between naïve and aGvHD mice. Each symbol represents an individual mouse. ***: p<0.001.

FIG. 5 shows that CXCR6-positive T cells are the major pathogenic T cells in aGvHD. IFNγ and GzmB double positive cells, a highly proinflammatory subpopulation, were exclusively CXCR6 positive in both CD4 and CD8 cells. Each symbol represents an individual mouse. *: p<0.05; **: p<0.01; ***: p<0.001.

FIG. 6 shows that CXCR6 is dispensable in aGvHD development. BALB/C mice that received either WT C57BL/6 or Cxcr6^{-/-} C57BL/6 donor cells developed equally severe aGvHD as judged by their similarly substantial and prolonged decrease of bodyweight, and deficiency of Cxcr6 on donor cells did not increase the survival.

FIG. 7 shows that anti-CXCR6 antibody treatment increased the survival of aGvHD mice. BALB/C mice which received WT C57BL/6 donor cells were treated with isotype control antibody or anti-CXCR6 antibody at indicated days and survival was monitored.

FIGS. 8A-8B show that CXCR6⁺ CD4 and CD8 T cells are enriched in tumor-infiltrating lymphocytes (TIL). FIG. 8A shows mean (±SEM) percent of CXCR6-expressing CD4 and CD8 T cells in TIL in B16 melanoma mice treated with isotype control mAb or anti-PD-1 mAb. FIG. 8B shows mean (±SEM) percent of CXCR6 expressing CD4 and CD8 T cells in TIL in the MC38 colon tumor mice. Symbols represent individual mice. *: p<0.05; **: p<0.01; ***: p<0.001.

FIGS. 9A-9B show that CXCR6-positive CD4 T cells are the major IFN-γ and GM-CSF producers in TIL in the B16 melanoma model. Compared with CXCR6-negative CD4 T cells, CXCR6-positive cells are more competent to produce IFN-γ and GM-CSF. FIG. 9A shows representative flow cytometry results. FIG. 9B shows statistical analysis of the data. Symbols represent individual mice. *: p<0.05; **: p<0.01.

FIGS. 10A-10B show that CXCR6-positive CD8 T cells are the major Granzyme B producers in TIL in the B16 melanoma model. Compared with CXCR6-negative CD8 T cells, CXCR6-positive cells are more competent to produce both IFN-γ and Granzyme B. FIG. 10A shows representative flow cytometry results. FIG. 10B shows statistical analysis of the data. Symbols represent individual mice. *: p<0.05; **: p<0.01.

FIG. 11 shows binding of nine candidate antibodies to cells overexpressing human, cyno monkey or mouse CXCR6. CNGs-1B7, CNE-6E10, COXs-1B10, COXs-2D2 and Beacon1-G12 bind to human CXCR6. Genovac1-G2, Beacon2-G5, Genovac2-C1 and Beacon1-B2 bind to both human and cyno monkey CXCR6.

FIG. 12A shows that human-specific mAbs bind to epitope residues within the extracellular N-terminus of human CXCR6. FIG. 12B shows that human/cyno-monkey cross-species mAbs do not bind to epitope residues within the extracellular N-terminus of human CXCR6.

FIG. 13 shows that human/cyno-monkey cross-species mAbs do not bind to epitope residues within the extracellular N-terminus of cyno-CXCR6.

FIGS. 14A and 14B show that COXs-1B10 but not Genovac2-C1 competed with R&D system-derived anti-human CXCR6 mAb for binding to human CXCR6. FIG. 14B is a color version of FIG. 14A.

FIGS. 15A and 15B show that chimeric antibodies retain the binding capability and high affinity of full-length murine IgG1. FIG. 15A shows that COXs-1B10 and Genovac2-C1 bind to human CXCR6. FIG. 15B shows that COXs-1B10 and Genovac2-C1 bind to cyno CXCR6.

FIG. 16 shows that binding of chimeric Genovac2-C1 to human CXCR6 over-expressing cells in the presence or absence of CXCL-16.

FIG. 17 shows antibody-dependent cellular cytotoxicity (ADCC) assay of human/mouse chimeric CXCR6 mAbs.

### DETAILED DESCRIPTION

A description of example embodiments follows.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The invention is not limited by the illustrated ordering of acts or events, as some acts may occur in different orders and/or concurrently with other acts or events. Furthermore, not all illustrated acts, steps or events are required to implement a methodology in accordance with the invention. Many of the techniques and procedures described, or referenced herein, are well understood and commonly employed using conventional methodology by those skilled in the art.

### CXCR6-Binding Polypeptides of the Disclosure

The present invention generally relates to polypeptides (e.g., antibodies, or antigen-binding fragments thereof) that bind to C-X-C motif chemokine receptor 6 (CXCR6) protein(s), and uses thereof. In one aspect, the invention provides a polypeptide that binds CXCR6, the polypeptide comprising heavy chain complementarity determining regions HCDR1, HCDR2 and HCDR3 that are at least 80% identical to the HCDR1, HCDR2 and HCDR3, respectively, of at least one immunoglobulin heavy chain variable region (V_{H}) set forth in SEQ ID NOs: 11-46. The sequences identified as SEQ ID NOs: 11-46 are shown in Table 2 herein.

The term “polypeptide,” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

The term “specifically binding” or “specifically binds” refers to preferential interaction, i.e., significantly higher binding affinity, e.g., between an antibody, or an antigen-binding fragment thereof, and its epitope relative to other antigens or amino acid sequences. In some embodiments, the polypeptide, e.g., antibody, specifically binds human CXCR6.

In some embodiments, the polypeptide, fusion protein or composition binds to a wild type CXCR6 protein, for example, wild type human CXCR6 (SEQ ID NO:1), wild type cynomolgus monkey (cyno) CXCR6 (SEQ ID NO:6), or both. The sequences identified as SEQ ID NO: 1 and SEQ ID NO:6 are shown in Table 1 herein.

In some embodiments, the polypeptide, fusion protein or composition binds to an extracellular N-terminus of a CXCR6 protein, e.g., the extracellular N-terminus of human CXCR6 (SEQ ID NO:2), the extracellular N-terminus of cyno CXCR6 (SEQ ID NO:7), or both. The sequences identified as SEQ ID NO:2 and SEQ ID NO:7 are shown in Table 1 herein.

In some embodiments, the polypeptide binds to one or more extracellular loops of a CXCR6 protein, e.g., extracellular loop 1 of human CXCR6 (SEQ ID NO:3), extracellular loop 1 of cyno CXCR6 (SEQ ID NO:8), extracellular loop 2 of human CXCR6 (SEQ ID NO:4), extracellular loop 2 of cyno CXCR6 (SEQ ID NO:9), extracellular loop 3 of human CXCR6 (SEQ ID NO:5), extracellular loop 3 of cyno CXCR6 (SEQ ID NO:10), or a combination thereof. The sequences identified as SEQ ID NOs:3-5 and 8-10 are shown in Table 1 herein.

In some embodiments, the polypeptide binds to a variant of CXCR6 protein comprising one or more amino acid substitutions, deletions and/or insertions relative to the wild type CXCR6 (e.g., relative to SEQ ID NO: 1 or SEQ ID NO:6). In some embodiments, the CXCR6 variant comprises an amino acid sequence that has at least about 90% sequence identity to the wild type CXCR6 sequence, for example, at least about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the wild type CXCR6 sequence. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%.

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. For instance, two sequences are considered to have 80% sequence identity when, upon alignment to achieve a maximal level of identity, the test sequence has the same nucleotide or amino acid residue at 80% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. See, for example, the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see, generally, Ausubel et al., Current Protocols in Molecular Biology).

In one example, when using a sequence comparison algorithm, test and reference sequences are input into a computer, subsequent coordinates are designated, if necessary, and sequence algorithm program parameters are designated. The sequence comparison algorithm then calculates the percent sequence identity for the test sequence (or test sequences) relative to the reference sequence, based on the designated program parameters. A commonly-used tool for determining percent sequence identity is Protein Basic Local Alignment Search Tool (BLASTP) available through National Center for Biotechnology Information, National Library of Medicine, of the United States National Institutes of Health (NIH) (See Altschul et al., 1990).

As used herein, the term “complementarity determining regions (CDRs)” refers to antigen binding sites in an antibody. CDRs may be defined using various terms: (i) HCDR1, HCDR2, HCDR3, LCDR1, LCDR2 and LCDR3, based on sequence variability (Wu and Kabat, J. Exp. Med. 132:211-50 (1970); Kabat et al., Sequences of Proteins of Immunological Interest, 5th Ed. Public Health Service, National Institutes of Health, Bethesda, Md. (1991)); (ii) “Hypervariable regions” (HVR or HV) H1, H2, H3, L1, L2 and L3, based on structure as defined by Chothia and Lesk (Chothia & Lesk, Mol. Biol. 196:901-17 (1987)); (iii) the International ImMunoGeneTics (IMGT) database (www_imgt_org) provides a standardized numbering and definition of antigen-binding sites. The correspondence between CDRs, HVs and IMGT delineations is described in Lefranc et al., Dev. Comparat. Immunol. 27:55-77 (2003). The terms “CDR”, “HCDR1”, “HCDR2”, “HCDR3”, “LCDR1”, “LCDR2” and “LCDR3” as used herein include CDRs defined by any of the methods described supra, in Kabat, Chothia and Lesk, or IMGT, unless explicitly stated otherwise. Tables 5 and 6 set forth the HCDR and LCDR amino acid sequences defined based on IMGT. Tables 7 and 8 set forth the HCDR and LCDR amino acid sequences defined based on Kabat et al. (see, e.g., http://www.bioinf.org.uk/abs/).

Substitution of one or more CDR residues or omission of one or more CDRs is possible. See, e.g., Padlan et al., Identification of specificity-determining residues in antibodies, FASEB J. 9(1): 133-9 (1995). Analysis of the contact regions between antibodies and their antigens, based on published crystal structures, led to the conclusion that only about one fifth to one third of CDR residues actually contact the antigen. One or more CDR residues may be omitted, or substituted with an amino acid occupying the corresponding position in another human antibody sequence or a consensus of such sequences. Positions for substitution and amino acids to substitute can be selected empirically.

In some embodiments, the polypeptide comprises HCDR1, HCDR2 and HCDR3 that are at least about 85% identical to the HCDR1, HCDR2 and HCDR3, respectively, of at least one immunoglobulin V_{H} set forth in SEQ ID NOs: 11-46. For example, the sequence identity can be at least about: 90%, 95%, 98% or 99%; or about: 85%, 86%, 87%, 88%, 89%, 90%, 91%, 92%, 93%, 94%, 95%, 96%, 97%, 98% or 99%. In some embodiments, the polypeptide comprises HCDR1, HCDR2 and HCDR3 that are at least about 85% (e.g., at least about 90%) identical to the HCDR1, HCDR2 and HCDR3, respectively, of at least one immunoglobulin V_{H} set forth in SEQ ID NO:11, SEQ ID NO:12, SEQ ID NO:13, SEQ ID NO:17, SEQ ID NO:19, SEQ ID NO:20 and SEQ ID NO:21 (e.g., SEQ ID NO: 11 or SEQ ID NO:20).

In some embodiments, the polypeptide comprises HCDR1, HCDR2 and HCDR3 that are identical to the HCDR1, HCDR2 and HCDR3, respectively, of an immunoglobulin V_{H} set forth in SEQ ID NOs:11-46. In some embodiments, the polypeptide comprises HCDR1, HCDR2 and HCDR3 that are identical to the HCDR1, HCDR2 and HCDR3, respectively, of an immunoglobulin V_{H} set forth in SEQ ID NO:11, SEQ ID NO: 12, SEQ ID NO:13, SEQ ID NO:17, SEQ ID NO:19, SEQ ID NO:20 and SEQ ID NO:21 (e.g., SEQ ID NO:11 or SEQ ID NO:20).

In some embodiments, the polypeptide further comprises LCDR1, LCDR2 and LCDR3 that are at least about 80% identical to the LCDR1, LCDR2 and LCDR3, respectively, of at least one immunoglobulin light chain variable region (V_{L}) set forth in SEQ ID NOs:47-82. For example, the sequence identity can be at least about: 85%, 90%, 95%, 98% or 99% or about: 81%, 82%, 83%, 84%, 85%, 86%, 87%, 88%, 89%, 90%, 91%, 92%, 93%, 94%, 95%, 96%, 97%, 98% or 99%. In some embodiments, the polypeptide further comprises LCDR1, LCDR2 and LCDR3 that are at least about 85% (e.g., at least about 90%) identical to the LCDR1, LCDR2 and LCDR3, respectively, of at least one immunoglobulin V_{L} set forth in SEQ ID NO:47, SEQ ID NO:48, SEQ ID NO:49, SEQ ID NO:53, SEQ ID NO:55, SEQ ID NO:56 and SEQ ID NO:57 (e.g., SEQ ID NO:47 or SEQ ID NO:56).

In some embodiments, the polypeptide comprises LCDR1, LCDR2 and LCDR3 that are identical to the LCDR1, LCDR2 and LCDR3, respectively, of an immunoglobulin V_{L} set forth in SEQ ID NOs:47-82. In some embodiments, the polypeptide comprises LCDR1, LCDR2 and LCDR3 that are identical to the LCDR1, LCDR2 and LCDR3, respectively, of an immunoglobulin V_{L} set forth in SEQ ID NO:47, SEQ ID NO:48, SEQ ID NO:49, SEQ ID NO:53, SEQ ID NO:55, SEQ ID NO:56 and SEQ ID NO:57 (e.g., SEQ ID NO:47 or SEQ ID NO:56).

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H} and a V_{L}, wherein the V_{H} and V_{L} of the antibody are selected from the following V_{H}/V_{L} combinations:
SEQ ID NO:11/SEQ ID NO:47 (Genovac2-C1);
SEQ ID NO:12/SEQ ID NO:48 (Genovac1-G2);
SEQ ID NO:13/SEQ ID NO:49 (Beacon2-G5);
SEQ ID NO:14/SEQ ID NO:50 (Beacon2-F1);
SEQ ID NO:15/SEQ ID NO:51 (CNE-6E5-C1);
SEQ ID NO:16/SEQ ID NO:52 (CNGs-3B2);
SEQ ID NO:17/SEQ ID NO:53 (Beacon1-B2);
SEQ ID NO:18/SEQ ID NO:54 (CNGs-1B7);
SEQ ID NO:19/SEQ ID NO:55 (CNE-6E10);
SEQ ID NO:20/SEQ ID NO:56 (COXs-1B10);
SEQ ID NO:21/SEQ ID NO:57 (COXs-2D2);
SEQ ID NO:22/SEQ ID NO:58 (Beacon1-D10);
SEQ ID NO:23/SEQ ID NO:59 (Beacon1-G12);
SEQ ID NO:24/SEQ ID NO:60 (COXs-1B11);
SEQ ID NO:25/SEQ ID NO:61 (COXs-2D4);
SEQ ID NO:23/SEQ ID NO:62 (COXs-4H5);
SEQ ID NO:26/SEQ ID NO:63 (COXs-7A7);
SEQ ID NO:27/SEQ ID NO:64 (COXs-7E6);
SEQ ID NO:28/SEQ ID NO:65 (COXs-9D6);
SEQ ID NO:29/SEQ ID NO:65 (COXs-11E5);
SEQ ID NO:30/SEQ ID NO:66 (COXs-11H4);
SEQ ID NO:31/SEQ ID NO:67 (COXs-12E7);
SEQ ID NO:32/SEQ ID NO:68 (CNE-7B1);
SEQ ID NO:33/SEQ ID NO:69 (CNF-2F8);
SEQ ID NO:34/SEQ ID NO:70 (CNGs-1C1);
SEQ ID NO:35/SEQ ID NO:71 (CNGs-2F11);
SEQ ID NO:36/SEQ ID NO:72 (CNE-6H7-G7);
SEQ ID NO:37/SEQ ID NO:73 (CNGs-1B7-E4);
SEQ ID NO:38/SEQ ID NO:74 (CNGs-1E1-G5);
SEQ ID NO:39/SEQ ID NO:75 (CNGs-3G6-G5);
SEQ ID NO:40/SEQ ID NO:76 (CNGs-3H2-B7);
SEQ ID NO:41/SEQ ID NO:77 (Beacon2-A3);
SEQ ID NO:42/SEQ ID NO:78 (Beacon2-A7);
SEQ ID NO:43/SEQ ID NO:69 (Beacon2-D5);
SEQ ID NO:44/SEQ ID NO:79 (Beacon2-E5);
SEQ ID NO:45/SEQ ID NO:80 (Genovac1-D12);
SEQ ID NO:18/SEQ ID NO:81 (Genovac2-H2); and
SEQ ID NO:46/SEQ ID NO:82 (Beacon1-A4).

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H} and a V_{L}, wherein the V_{H} and V_{L} of the antibody are selected from the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:17/SEQ ID NO:53;
SEQ ID NO:19/SEQ ID NO:55;
SEQ ID NO:20/SEQ ID NO:56; or
SEQ ID NO:21/SEQ ID NO:57.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO: 11/SEQ ID NO:47.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:12/SEQ ID NO:48.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:13/SEQ ID NO:49.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:17/SEQ ID NO:53.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:19/SEQ ID NO:55.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:20/SEQ ID NO:56.

In some embodiments, the polypeptide comprises HCDR1, HCDR2 and HCDR3, and LCDR1, LCDR2 and LCDR3, that are at least about 80% identical (e.g., at least about: 85%, 90%, 95% or 99% identical, or identical) to the HCDR1, HCDR2 and HCDR3, and LCDR1, LCDR2 and LCDR3, respectively, of an antibody comprising a V_{H}/V_{L} combination set forth in SEQ ID NO:21/SEQ ID NO:57.

In some embodiments, the polypeptide is an immunoglobulin molecule that comprises a V_{H} and a V_{L}.

In some embodiments, the polypeptide comprises:
a) a V_{H} that is at least about 60% identical to at least one sequence set forth in SEQ ID NOs: 11-46;
b) a V_{L} that is at least about 60% identical to at least one sequence set forth in SEQ ID NOs:47-82; or
c) both a) and b).

In some embodiments, the polypeptide comprises a V_{H} that is at least about 60% identical to at least one sequence set forth in SEQ ID NOs: 11-46. For example, sequence identity can be at least about: 65%, 70%, 75%, 80%, 85%, 90% or 95%, or about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some embodiments, the polypeptide comprises a V_{H} that is at least about 60% (e.g., at least about: 70%, 80%, 90% or 95%) identical to at least one sequence set forth in SEQ ID NO:11, SEQ ID NO: 12, SEQ ID NO: 13, SEQ ID NO: 17, SEQ ID NO: 19, SEQ ID NO:20 or SEQ ID NO:21 (e.g., SEQ ID NO:11 or SEQ ID NO:20).

In some embodiments, the polypeptide comprises a V_{L} that is at least about 60% identical to at least one sequence set forth in SEQ ID NOs:47-82. For example, sequence identity can be at least about: 65%, 70%, 75%, 80%, 85%, 90% or 95%, or about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some embodiments, the polypeptide comprises a V_{L} that is at least about 60% (e.g., at least about: 70%, 80%, 90% or 95%) identical to at least one sequence set forth in SEQ ID NO:47, SEQ ID NO:48, SEQ ID NO:49, SEQ ID NO:53, SEQ ID NO:55, SEQ ID NO:56 and SEQ ID NO:57 (e.g., SEQ ID NO:47 or SEQ ID NO:56).

In some embodiments, the polypeptide comprises:
a) a V_{H} comprising one or more amino acid substitutions relative to at least one sequence set forth in SEQ ID NOs: 11-46;
b) a V_{L} comprising one or more amino acid substitutions relative to at least one sequence set forth in SEQ ID NOs:47-82; or
c) both a) and b).

In some embodiments, the polypeptide comprises a V_{H} comprising one or more amino acid substitutions relative to at least one sequence set forth in SEQ ID NOs: 11-46. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the V_{H} comprises about 1-20, 1-15, 1-10 or 1-5 amino acid substitutions, relative to at least one sequence set forth in SEQ ID NOs:11-46. In some embodiments, the V_{H} comprises about 1-20, 1-15, 1-10 or 1-5 amino acid substitutions, relative to at least one sequence set forth in SEQ ID NO:11, SEQ ID NO: 12, SEQ ID NO:13, SEQ ID NO: 17, SEQ ID NO: 19, SEQ ID NO:20 or SEQ ID NO:21 (e.g., SEQ ID NO:11 or SEQ ID NO:20).

In some embodiments, the polypeptide comprises a V_{L} comprising one or more amino acid substitutions relative to at least one sequence set forth in SEQ ID NOs:47-82. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the V_{L} comprises about 1-20, 1-15, 1-10 or 1-5 amino acid substitutions, relative to at least one sequence set forth in SEQ ID NOs:47-82. In some embodiments, the V_{L} comprises about 1-20, 1-15, 1-10 or 1-5 amino acid substitutions, relative to at least one sequence set forth in SEQ ID NO:47, SEQ ID NO:48, SEQ ID NO:49, SEQ ID NO:53, SEQ ID NO:55, SEQ ID NO:56 and SEQ ID NO:57 (e.g., SEQ ID NO:47 or SEQ ID NO:56).

In some embodiments, the one or more amino acid substitutions are conservative substitutions. As used herein, the term “conservative amino acid substitution(s)” or “conservative substitution(s)” refers to an amino acid substitution having a value of 0 or greater in the BLOSUM62 matrix for amino acid substitutions.

In some embodiments, the one or more amino acid substitutions are highly conservative substitutions. As used herein, the term “highly conservative amino acid substitution(s)” or “highly conservative substitution(s)” refers to an amino acid substitution having a value of at least 1 (e.g., 2 or more) in BLOSUM62.

In some embodiments, the polypeptide comprises:
a) a V_{H} comprising a sequence set forth in any one of SEQ ID NOs: 11-46;
b) a V_{L} comprising a sequence set forth in any one of SEQ ID NOs:47-82; or
c) both a) and b).

In some embodiments, the polypeptide comprises a V_{H}/V_{L} combination that is identical to any one of the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:14/SEQ ID NO:50;
SEQ ID NO:15/SEQ ID NO:51;
SEQ ID NO:16/SEQ ID NO:52;
SEQ ID NO:17/SEQ ID NO:53;
SEQ ID NO:18/SEQ ID NO:54;
SEQ ID NO:19/SEQ ID NO:55;
SEQ ID NO:20/SEQ ID NO:56;
SEQ ID NO:21/SEQ ID NO:57;
SEQ ID NO:22/SEQ ID NO:58;
SEQ ID NO:23/SEQ ID NO:59;
SEQ ID NO:24/SEQ ID NO:60;
SEQ ID NO:25/SEQ ID NO:61;
SEQ ID NO:23/SEQ ID NO:62;
SEQ ID NO:26/SEQ ID NO:63;
SEQ ID NO:27/SEQ ID NO:64;
SEQ ID NO:28/SEQ ID NO:65;
SEQ ID NO:29/SEQ ID NO:65;
SEQ ID NO:30/SEQ ID NO:66;
SEQ ID NO:31/SEQ ID NO:67;
SEQ ID NO:32/SEQ ID NO:68;
SEQ ID NO:33/SEQ ID NO:69;
SEQ ID NO:34/SEQ ID NO:70;
SEQ ID NO:35/SEQ ID NO:71;
SEQ ID NO:36/SEQ ID NO:72;
SEQ ID NO:37/SEQ ID NO:73;
SEQ ID NO:38/SEQ ID NO:74;
SEQ ID NO:39/SEQ ID NO:75;
SEQ ID NO:40/SEQ ID NO:76;
SEQ ID NO:41/SEQ ID NO:77;
SEQ ID NO:42/SEQ ID NO:78;
SEQ ID NO:43/SEQ ID NO:69;
SEQ ID NO:44/SEQ ID NO:79;
SEQ ID NO:45/SEQ ID NO:80;
SEQ ID NO:18/SEQ ID NO:81; and
SEQ ID NO:46/SEQ ID NO:82.

In some embodiments, the polypeptide comprises a V_{H}/V_{L} combination that is identical to any one of the following V_{H}/V_{L} combinations:
SEQ ID NO:11/SEQ ID NO:47;
SEQ ID NO:12/SEQ ID NO:48;
SEQ ID NO: 13/SEQ ID NO:49;
SEQ ID NO:17/SEQ ID NO:53;
SEQ ID NO:19/SEQ ID NO:55;
SEQ ID NO:20/SEQ ID NO:56; and
SEQ ID NO:21/SEQ ID NO:57.

In some embodiments, the polypeptide comprises a V_{H}/V_{L} combination set forth in SEQ ID NO:11/SEQ ID NO:47. In some embodiments, the polypeptide comprises a V_{H}/V_{L} combination set forth in SEQ ID NO:12/SEQ ID NO:48. In some embodiments, the polypeptide comprises a V_{H}/V_{L} combination set forth in SEQ ID NO:13/SEQ ID NO:49. In some embodiments, the polypeptide comprises a V_{H}/V_{L} combination set forth in SEQ ID NO:17/SEQ ID NO:53. In some embodiments, the polypeptide comprises a V_{H}/V_{L} combination set forth in SEQ ID NO:19/SEQ ID NO:55. In some embodiments, the polypeptide comprises a V_{H}/V_{L} combination set forth in SEQ ID NO:20/SEQ ID NO:56. In some embodiments, the polypeptide comprises a V_{H}/V_{L} combination set forth in SEQ ID NO:21/SEQ ID NO:57.

In some embodiments, the polypeptide further comprises:
a) an antibody heavy chain constant region sequence;
b) an antibody light chain constant region sequence; or
c) both a) and b).

In some embodiments, the polypeptide is an immunoglobulin molecule, such as an antibody (e.g., a whole antibody, an intact antibody) or an antigen-binding fragment thereof.

Immunoglobulins may be assigned to five major classes: IgA, IgD, IgE, IgG and IgM, depending on the heavy chain constant domain amino acid sequence. IgA is further sub-classified as the isotypes IgA₁, IgA₂. IgG is further sub-classified as IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

In some embodiments, the antibody heavy chain constant region is selected from the group consisting of an IgA constant region, an IgD constant region, an IgE constant region, an IgG constant region and an IgM constant region. In some embodiments, the antibody heavy chain constant region is an IgG constant region. In some embodiments, the IgG constant region is an IgG1 constant region, an IgG2 constant region, an IgG3 constant region or an IgG4 constant region. In some embodiments, the IgG2 constant region is an IgG2a, an IgG2b constant region or an IgG₂c constant region. In some embodiments, the antibody heavy chain constant region is an IgG1 constant region (e.g., IGHV5-51). In some embodiments, the antibody heavy chain constant region is an IgG4 constant region.

In some embodiments, the polypeptide disclosed herein is an antibody. The term “antibody” refers to an immunoglobulin molecule capable of specific binding to a target, such as a polypeptide, carbohydrate, polynucleotide or lipid, through at least one antigen recognition site, located in the variable region of the immunoglobulin molecule. As used herein, the term “antibody” refers to a full-length antibody comprising two heavy (H) chains and two light (L) chains inter-connected by disulfide bonds or multimers thereof (for example, IgM). Each heavy chain comprises a heavy chain variable region (V_{H}) and a heavy chain constant region (comprising domains CH1, hinge CH2 and CH3). Each light chain comprises a light chain variable region (V_{L}) and a light chain constant region (CL). The V_{H} and the V_{L} regions may be further subdivided into regions of hypervariability, termed CDRs, interspersed within framework regions (FR). V_{H} and V_{L} each comprise three CDRs and four FR segments, arranged from the amino-terminus to the carboxy-terminus in the following order: FR1, CDR1, FR2, CDR2, FR3, CDR3, and FR4.

In some embodiments, the polypeptide is a monoclonal antibody. “Monoclonal antibody” refers to an antibody population with a single amino acid composition in each heavy and each light chain, except for possible well-known alterations such as removal of C-terminal lysine from the antibody heavy chain. Monoclonal antibodies may have heterogeneous glycosylation within the antibody population. A monoclonal antibody may be monovalent, bivalent or multivalent. A monoclonal antibody may be monospecific (binds one antigenic epitope) or multispecific (binds two (bispecific) or more distinct antigens or epitopes).

In some embodiments, the polypeptide is a bispecific antibody that binds an immune checkpoint protein and CXCR6. In some embodiments, the polypeptide is a bispecific antibody that binds programmed cell death protein 1 (PD-1) and CXCR6. In some embodiments, the polypeptide is a bispecific antibody that binds programmed death-ligand 1 (PD-L1) and CXCR6.

In some embodiments, the polypeptide is an isolated antibody or an antigen-binding fragment thereof, i.e., is substantially free of other antibodies (e.g., antibodies that do not specifically bind a CXCR6 protein). In some embodiments, the anti-CXCR6 antibody or antigen-binding fragment thereof is at least 80% pure, e.g., about: 80%, 81%, 82%, 83%, 84%, 85%, 86%, 87%, 88%, 89%, 90%, 91%, 92%, 93%, 94%, 95%, 96%, 97%, 98%, 99% or 100% pure.

The antibody can be of any species, such as a murine antibody or a human antibody. In some embodiments, the polypeptide is a chimeric antibody, a humanized antibody or a human antibody. In some embodiments, the polypeptide is a human antibody.

In some embodiments, the V_{H}, the V_{L} or both, of the polypeptide, comprise one or more human framework regions.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of “human antibody.”

As will be appreciated by a person of ordinary skill in the art, an antibody having a particular Fc isotype can be converted to an antibody with a different Fc isotype. For example, an antibody with a mouse IgG1 Fc can be converted to an antibody with a human IgG4 without altering variable domains, and the binding properties to antigen are expected to be identical or substantially similar regardless of the nature of the Fc domain. In some embodiments, the IgG4 Fc domain comprises two or more amino acid changes as disclosed in US Pat. Publ. No. US20100331527. In some embodiments, the human IgG4 Fc comprises a serine to proline mutation in the hinge region (S108P) to promote dimer stabilization.

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90%, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immuno-globulin gene libraries displayed on phage (see, e.g., Shi et al., J. Mol. Biol. 397:385-96 (2010) and Int. Pat. Publ. No. WO2009/085462).

In some embodiments, the antibody heavy chain constant region sequence is at least about 60% identical to human IgG1 (e.g., SEQ ID NO:345) or human IgG4 (e.g., SEQ ID NO:346). For example, the antibody heavy chain constant region sequence can be at least about: 65%, 70%, 75%, 80%, 85%, 90% or 95%; or about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to human IgG1 (e.g., SEQ ID NO:345) or human IgG4 (e.g., SEQ ID NO:346). In some embodiments, the antibody heavy chain constant region sequence is identical to human IgG1 (e.g., SEQ ID NO:345) or human IgG4 (e.g., SEQ ID NO:346). The sequences identified as SEQ ID NO:345 and SEQ ID NO:346 are shown below:

(SEQ ID NO: 345)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.
(SEQ ID NO: 346)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In some embodiments, the antibody heavy chain constant region sequence comprises one or more amino acid substitutions relative to human IgG1 (e.g., SEQ ID NO:345) or human IgG4 (e.g., SEQ ID NO:346). For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the antibody heavy chain constant region sequence comprises about 1-10 amino acid substitutions, relative to human IgG1 (e.g., SEQ ID NO:345) or human IgG4 (e.g., SEQ ID NO:346). In some embodiments, the one or more amino acid substitutions are conservative substitutions. In some embodiments, the one or more amino acid substitutions are highly conservative substitutions.

In some embodiments, the antibody light chain constant region is selected from the group consisting of a κ constant region and a À constant region. In some embodiments, the antibody light chain constant region is a k constant region.

In some embodiments, the antibody light chain constant region sequence is at least about 60% identical to SEQ ID NO:347 or SEQ ID NO:348. For example, the antibody light chain constant region sequence can be at least about: 65%, 70%, 75%, 80%, 85%, 90% or 95%; or about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:347 or SEQ ID NO:348. In some embodiments, the antibody light chain constant region sequence is identical to SEQ ID NO:347. In some embodiments, the antibody light chain constant region sequence is identical to SEQ ID NO:348. The sequences identified as SEQ ID NO:347 or SEQ ID NO:348 are shown below:

(SEQ ID NO: 347)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.
(SEQ ID NO: 348)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS.

In some embodiments, the antibody light chain constant region sequence comprises one or more amino acid substitutions relative to SEQ ID NO:347 or SEQ ID NO:348. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the antibody light chain constant region sequence comprises about 1-10 amino acid substitutions, relative to SEQ ID NO:347 or SEQ ID NO:348. In some embodiments, the one or more amino acid substitutions are conservative substitutions. In some embodiments, the one or more amino acid substitutions are highly conservative substitutions.

In some embodiments, the antibody heavy chain constant region is an IgG1 constant region, and the antibody light chain constant region is a κ constant region. In some embodiments, the antibody heavy chain constant region is an IgG1 constant region, and the antibody light chain constant region is a λ constant region.

In some embodiments, the polypeptide disclosed herein is a fragment, e.g., an antigen-binding fragment. The term “antigen-binding fragment” refers to a portion of an immunoglobulin molecule (e.g., an antibody) that retains the antigen binding properties of the parental full-length antibody. Non-limiting examples of antigen-binding fragments include LCDR1, 2 and/or 3, HCDR1, 2 and/or 3, a V_{H} region, a V_{L} region, an Fab fragment, an F(ab′)2 fragment, an Fd fragment, an Fv fragment, and a domain antibody (dAb) consisting of one V_{H} domain or one V_{L} domain, etc. V_{H} and V_{L} domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody. See, for example, Int. Pat. Publ. Nos. WO1998/44001, WO1988/01649, WO1994/13804 and WO1992/01047, the contents of which are incorporated herein in their entirety. In some embodiments, the polypeptide disclosed herein is an antigen binding fragment selected from Fab, F(ab′)2, Fab′, scFv, or Fv. In some embodiments, the polypeptide is a scFv.

In some embodiments, the polypeptide is an antibody mimetic. The term “antibody mimetic” refers to a polypeptide capable of mimicking an antibody's ability to bind an antigen, but structurally differ from native antibody structures. Non-limiting examples of antibody mimetics include Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

In some embodiments, the polypeptide is recombinantly produced. “Recombinant” includes antibodies and other proteins that are prepared, expressed, created or isolated by recombinant means.

In some embodiments, the polypeptide is synthetically produced.

In some embodiments, the polypeptide is conjugated to a heterologous moiety. The term “conjugated” refers to attached, via a covalent or noncovalent interaction. Conjugation can employ any suitable linking agent. Non-limiting examples include peptide linkers, compound linkers, and chemical cross-linking agents.

In some embodiments, the heterologous moiety is a therapeutic agent, a diagnostic agent or a combination thereof.

In some embodiments, the heterologous moiety is a diagnostic agent. In some embodiments, the diagnostic agent comprises a detectable label, a reporter molecule, or both. Non-limiting examples of diagnostic agents include radioisotopes (e.g., ³H, ¹⁴C, ³²P, ³⁵S, or ¹²⁵I), fluorescent moieties (e.g., fluorescein isothiocyanate, or rhodamine), chemiluminescent moieties and enzymes (e.g., alkaline phosphatase, β-galactosidase, horseradish peroxidase, or luciferase).

In some embodiments, the heterologous moiety comprises a human proinflammatory mediator, a human cytokine, or a combination thereof. In some embodiments, the heterologous moiety comprises a human proinflammatory mediator. Non-limiting examples of human proinflammatory mediators include perforin-A and granzyme A (GzmA). In some embodiments, the heterologous moiety comprises a cytokine, e.g., a human cytokine. Non-limiting examples of human cytokines include tumor necrosis factor α (TNF-α), interleukin (IL)-17 (IL-17), IL-6, IL-12, IL-21, IL-23, interferon gamma (IFNγ), granulocyte-macrophage colony-stimulating factor (GM-CSF)), IL-2, IL-7 and IL-15. In some embodiments, the heterologous moiety comprises TNF-α, IL-17, IL-6, IL-12, IL-21, IL-23, IFNγ or GM-CSF, or a combination thereof. In some embodiments, the heterologous moiety comprises IL-2, IL-7 or IL-15, or a combination thereof.

In some embodiments, the heterologous moiety is polyethylene glycol (PEG), ahexadecanoic acid, nanoparticles, hydrogel(s), multimerization domain(s) and/or carrier peptide(s). In some embodiments, the nanoparticle is a lipid nanoparticle. In some embodiments, the nanoparticle is a polymer nanoparticle. In some embodiments, the polymer is an amphiphilic polymer. In other embodiments, the polymer is a hydrophobic or hydrophilic polymer. Examples of polymers include, but are not limited to: poly(lactic acid)-poly(ethylene glycol), poly(lactic-co-glycolic acid)-poly(ethylene glycol), poly(lactic-co-glycolic) acid (PLGA), poly(lactic-co-glycolic acid)-d-α-tocopheryl polyethylene glycol succinate, poly(lactic-co-glycolic acid)-ethylene oxide fumarate, polycaprolactone-poly(ethylene glycol), poly(glycolic acid)-poly(ethylene glycol), or any salts thereof. In some embodiments, the polymer nanoparticle comprises poly(lactic-co-glycolic) acid (PLGA).

In some embodiments, the polypeptide is a “neutralizing antibody.” As used herein, the term “neutralizing antibody” refers to an antibody whose binding to CXCR6 results in inhibiting at least one biological activity of CXCR6. For example, an antibody of the invention may block the chemotaxis function at a certain environment such as NK T cells in liver pathology.

### Fusion Proteins

In another aspect, the invention provides a fusion protein comprising one or more of the polypeptides described herein.

The term “fusion protein” refers to a synthetic, semi-synthetic or recombinant single protein molecule. A fusion protein can comprise all or a portion of two or more different proteins and/or polypeptides that are attached by covalent bonds (e.g., peptide bonds).

Fusion proteins of the invention can be produced, e.g., recombinantly or synthetically, using routine methods and reagents that are well known in the art. For example, a fusion protein of the invention can be produced recombinantly in a suitable host cell (e.g., a bacterial cell) according to methods known in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press. For example, a nucleic acid molecule comprising a nucleotide sequence encoding a fusion protein described herein can be introduced and expressed in a suitable host cell (e.g., E. coli), and the expressed fusion protein can be isolated/purified from the host cell (e.g., in inclusion bodies) using routine methods and readily available reagents. For example, DNA fragments coding for different protein sequences (e.g., a light-responsive domain, a heterologous peptide component) can be ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

### Nucleic Acids, Expression Vectors, Expression Host Cells

In another aspect, the invention provides one or more polynucleotides encoding any one of the polypeptides or fusion proteins described herein. In some embodiments, the polypeptide or fusion protein of the invention is encoded by a single polynucleotide. In some embodiments, the polypeptide or fusion protein of the invention is encoded by multiple polynucleotides. Non-limiting examples of polynucleotides include linear deoxyribonucleic acid (DNA), linear ribonucleic acid (RNA), circular DNA and circular RNA, etc.

In some embodiments, the polynucleotide comprises a nucleotide sequence that is codon-optimized for a chosen host cell.

In another aspect, the invention provides an expression vector comprising any one or more of the polynucleotides described herein.

The term “expression vector” refers to a replicable nucleic acid from which one or more proteins can be expressed when the expression vector is transformed into a suitable expression host cell.

In some embodiments, the expression vector further comprises an expression control polynucleotide sequence operably linked to the polynucleotide, a polynucleotide sequence encoding a selectable marker, or both. In some embodiments, the expression control polynucleotide sequence comprises a promoter sequence, an enhancer sequence, or both. In some embodiments, the expression control polynucleotide sequence comprises an inducible promoter sequence. The term “promoter” refers to a region of DNA to which RNA polymerase binds and initiates the transcription of a gene. The term “operably linked” means that the nucleic acid is positioned in the recombinant polynucleotide, e.g., vector, in such a way that enables expression of the nucleic acid under control of the element (e.g., promoter) to which it is linked. The term “selectable marker element” is an element that confers a trait suitable for artificial selection. Selectable marker elements can be negative or positive selection markers.

In another aspect, the invention provides an expression host cell comprising any one or more of the polynucleotides or expression vectors described herein.

The term “expression host cell” refers to a cell useful for receiving, maintaining, reproducing and amplifying a vector. Non-limiting examples of expression host cells include mammalian cells such as hybridoma cells, Chinese hamster ovary (CHO) cells, COS cells, human embryonic kidney (HEK), yeast cells such as Pichia pastoris cells, or bacterial cells such as DH5α, etc.

### Compositions

In another aspect, the invention provides a composition comprising one or more of the polypeptides, fusion proteins, polynucleotides, expression vectors or host cells described herein. In some embodiments, the composition comprises one or more of the polypeptides or fusion proteins described herein. In some embodiments, the composition is a pharmaceutical composition.

In some embodiments, the composition (e.g., pharmaceutical composition) further comprises pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

Various delivery systems can be used to administer a pharmaceutical composition of the invention, e.g., encapsulation in liposomes (see, e.g., Langer, New methods of drug delivery, Science 249(4976): 1527-33 (1990)), microparticles, microcapsules, recombinant cells capable of expressing the mutant viruses, receptor mediated endocytosis (see, e.g., Wu & Wu, Receptor-mediated in vitro gene transformation by a soluble DNA carrier system, J Biol Chem. 262(10):4429-32 (1987)). The use of nanoparticles to deliver a composition (e.g., pharmaceutical composition) of the present invention is also contemplated herein. For additional information on antibody-conjugated nanoparticles and methods of preparation and use, see, e.g., Arruebo et al., Antibody-Conjugated Nanoparticles for Biomedical Applications, Journal of Nanomaterials (2009) and U.S. Pat. Nos. 8,257,740, and 8,246,995, the contents of which are incorporated herein in their entirety.

In some embodiments, a composition (e.g., pharmaceutical composition) of the invention is formulated for a suitable administration route. Non-limiting examples of administration routes include oral, nasal, vaginal, rectal, mucosal, intravenous, intramuscular, subcutaneous, intradermal, intraperitoneal, pulmonary, topical and transmucosal (oral, intranasal, intravaginal or rectal). In some embodiments, the composition is formulated for intravenous administration. In some embodiments, the composition is formulated for subcutaneous administration. In some embodiments, the composition (e.g., pharmaceutical composition) is stored in the form of an aqueous solution or a dried formulation (e.g., lyophilized). In some embodiments, non-limiting examples of administration routes include oral, nasal, vaginal, rectal, mucosal, intravenous, intramuscular, subcutaneous and topical.

In some embodiments, the composition (e.g., pharmaceutical composition) is formulated to be administered with an additional therapeutic agent (e.g., a second therapeutic agent) as a combination therapy.

In some embodiments, the composition (e.g., pharmaceutical composition) comprises a polypeptide comprising a V_{H}/V_{L} combination of:
SEQ ID NO:11/SEQ ID NO:47;
SEQ ID NO:12/SEQ ID NO:48;
SEQ ID NO:13/SEQ ID NO:49;
SEQ ID NO:14/SEQ ID NO:50;
SEQ ID NO:15/SEQ ID NO:51;
SEQ ID NO:16/SEQ ID NO:52;
SEQ ID NO:17/SEQ ID NO:53;
SEQ ID NO:18/SEQ ID NO:54;
SEQ ID NO:19/SEQ ID NO:55;
SEQ ID NO:20/SEQ ID NO:56;
SEQ ID NO:21/SEQ ID NO:57;
SEQ ID NO:22/SEQ ID NO:58;
SEQ ID NO:23/SEQ ID NO:59;
SEQ ID NO:24/SEQ ID NO:60;
SEQ ID NO:25/SEQ ID NO:61;
SEQ ID NO:23/SEQ ID NO:62;
SEQ ID NO:26/SEQ ID NO:63;
SEQ ID NO:27/SEQ ID NO:64;
SEQ ID NO:28/SEQ ID NO:65;
SEQ ID NO:29/SEQ ID NO:65;
SEQ ID NO:30/SEQ ID NO:66;
SEQ ID NO:31/SEQ ID NO:67;
SEQ ID NO:32/SEQ ID NO:68;
SEQ ID NO:33/SEQ ID NO:69;
SEQ ID NO:34/SEQ ID NO:70;
SEQ ID NO:35/SEQ ID NO:71;
SEQ ID NO:36/SEQ ID NO:72;
SEQ ID NO:37/SEQ ID NO:73;
SEQ ID NO:38/SEQ ID NO:74;
SEQ ID NO:39/SEQ ID NO:75;
SEQ ID NO:40/SEQ ID NO:76;
SEQ ID NO:41/SEQ ID NO:77;
SEQ ID NO:42/SEQ ID NO:78;
SEQ ID NO:43/SEQ ID NO:69;
SEQ ID NO:44/SEQ ID NO:79;
SEQ ID NO:45/SEQ ID NO:80;
SEQ ID NO:18/SEQ ID NO:81; or
SEQ ID NO:46/SEQ ID NO:82.

In some embodiments, the composition (e.g., pharmaceutical composition) comprises a polypeptide comprising a V_{H}/V_{L} combination of:
SEQ ID NO:11/SEQ ID NO:47;
SEQ ID NO:12/SEQ ID NO:48;
SEQ ID NO:13/SEQ ID NO:49;
SEQ ID NO:17/SEQ ID NO:53;
SEQ ID NO:19/SEQ ID NO:55;
SEQ ID NO:20/SEQ ID NO:56; or
SEQ ID NO:21/SEQ ID NO:57.

In some embodiments, the composition (e.g., pharmaceutical composition) comprises a polypeptide comprising a V_{H}/V_{L} combination of SEQ ID NO: 11/SEQ ID NO:47. In some embodiments, the composition (e.g., pharmaceutical composition) comprises a polypeptide comprising a V_{H}/V_{L} combination of SEQ ID NO: 12/SEQ ID NO:48. In some embodiments, the composition (e.g., pharmaceutical composition) comprises a polypeptide comprising a V_{H}/V_{L} combination of SEQ ID NO: 13/SEQ ID NO:49. In some embodiments, the composition (e.g., pharmaceutical composition) comprises a polypeptide comprising a V_{H}/V_{L} combination of SEQ ID NO:17/SEQ ID NO:53. In some embodiments, the composition (e.g., pharmaceutical composition) comprises a polypeptide comprising a V_{H}/V_{L} combination of SEQ ID NO: 19/SEQ ID NO:55. In some embodiments, the composition (e.g., pharmaceutical composition) comprises a polypeptide comprising a V_{H}/V_{L} combination of SEQ ID NO:20/SEQ ID NO:56. In some embodiments, the composition (e.g., pharmaceutical composition) comprises a polypeptide comprising a V_{H}/V_{L} combination of SEQ ID NO:21/SEQ ID NO:57.

### Methods of Use

In another aspect, the invention provides a method of modulating a CXCR6-positive leukocyte, comprising contacting the CXCR6-positive leukocyte with one or more of the polypeptides, fusion proteins or compositions described herein, thereby modulating the CXCR6-positive leukocyte.

In some embodiments, the CXCR6-positive leukocyte comprises CD4 T cells, CD8 T cells, Gamma delta (γδ) T cells, natural killer (NK) T cells, natural killer (NK) cells or neutrophils, or combinations thereof.

In some embodiments, the method comprises contacting the CXCR6-positive leukocyte with a polypeptide of the invention (e.g., an anti-CXCR6 antibody) at a concentration of at least about 0.01 μg/ml. For example, in some embodiments, the concentration is at least about: 0.02 μg/ml, 0.03 μg/ml, 0.04 μg/ml, 0.05 μg/ml, 0.10 μg/ml, 0.25 μg/ml, 0.50 μg/ml, 0.75 μg/ml, 1 μg/ml, 2 μg/ml, 3 μg/ml, 4 μg/ml, 5 μg/ml, 6 μg/ml, 7 μg/ml, 8 μg/ml, 9 g/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 25 μg/ml, 30 μg/ml, 40 μg/ml, 50 μg/ml or 100 g/ml; or about: 0.01-100 μg/ml, 0.02-100 μg/ml, 0.02-50 g/ml, 0.03-50 μg/ml, 0.03-40 μg/ml, 0.04-40 μg/ml, 0.04-30 μg/ml, 0.05-30 μg/ml or 0.05-20 μg/ml.

In some embodiments, the method comprises contacting the CXCR6-positive leukocyte with a polypeptide of the invention (e.g., an anti-CXCR6 antibody) for at least about 1 minute, e.g., at least about: 2 minutes, 3 minutes, 4 minutes, 5 minutes, 6 minutes, 7 minutes, 8 minutes, 9 minutes, 10 minutes, 12 minutes, 15 minutes, 20 minutes, 25 minutes, 30 minutes, 35 minutes, 40 minutes, 45 minutes, 50 minutes, 55 minutes, 60 minutes, 2 hours, 3 hours, 4 hours, 5 hours, 6 hours, 7 hours, 8 hours, 9 hours, 10 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 5 days, 6 days, 7 days, 8 days, 9 days or 10 days. In some embodiments, the method comprises contacting the CXCR6-positive leukocyte with a polypeptide of the invention (e.g., an anti-CXCR6 antibody) for about 1 minute to 10 days, for example, 3 minutes to 10 days, 3 minutes to 8 days, 10 minutes to 8 days, 10 minutes to 6 days, 15 minutes to 6 days, 15 minutes to 4 days, 30 minutes to 4 days, 30 minutes to 48 hours, 45 minutes to 48 hours, 45 minutes to 36 hours, 1-36 hours, 1-24 hours, 2-24 hours, 2-18 hours, 3-18 hours, 3-12 hours, 4-12 hours or 4-6 hours.

In some embodiments, the method kills the CXCR6-positive leukocyte. In some embodiments, the method kills the CXCR6-positive leukocyte by antibody-dependent cellular cytotoxicity (ADCC). In some embodiments, the method kills the CXCR6-positive leukocyte by complement-dependent cytotoxicity (CDC). In some embodiments, the method kills the CXCR6-positive leukocyte by antibody-dependent cellular phagocytosis (ADCP). In some embodiments, the method kills the CXCR6-positive leukocyte by altering intermediate metabolism and/or inducing necrosis. In some embodiments, the method kills the CXCR6-positive leukocyte by ADCC, CDC or ADCP, or inducing necrosis, or a combination thereof.

In some embodiments, the method decreases (e.g., inhibits or blocks) chemotaxis of the CXCR6-positive leukocyte.

In some embodiments, the method inactivates the CXCR6-positive leukocyte (e.g., via signal transduction). In some embodiments, the method of inactivating the CXCR6-positive leukocyte comprises contacting the CXCR6-positive leukocyte with a polypeptide of the invention (e.g., an anti-CXCR6 antibody) at a concentration of about 0.05-20 μg/ml, e.g., for several minutes up to several days following the usual range of antibody's half-life in human body. Antibody half-life in humans varies, ranging from e.g., a couple of days to longer than 10 days, depending on, for example, the dose (a higher dose can significantly prolong the half-life) and the immunogenicity of antibody (a presence of an anti-drug-antibody can significantly shorten the half-life). In some embodiments, the method of inactivating the CXCR6-positive leukocyte comprises contacting the CXCR6-positive leukocyte with a polypeptide of the invention (e.g., an anti-CXCR6 antibody) for at least about 1 minute, e.g., at least about: 2 minutes, 3 minutes, 4 minutes, 5 minutes, 6 minutes, 7 minutes, 8 minutes, 9 minutes, 10 minutes, 12 minutes, 15 minutes, 20 minutes, 25 minutes, 30 minutes, 35 minutes, 40 minutes, 45 minutes, 50 minutes, 55 minutes, 60 minutes, 2 hours, 3 hours, 4 hours, 5 hours, 6 hours, 7 hours, 8 hours, 9 hours, 10 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 5 days, 6 days, 7 days, 8 days, 9 days or 10 days. In some embodiments, the method of inactivating the survival of the CXCR6-positive leukocyte comprises contacting the CXCR6-positive leukocyte with a polypeptide of the invention (e.g., an anti-CXCR6 antibody) for about 1 minute to 10 days, for example, 3 minutes to 10 days, 3 minutes to 8 days, 10 minutes to 8 days, 10 minutes to 6 days, 15 minutes to 6 days, 15 minutes to 4 days, 30 minutes to 4 days, 30 minutes to 48 hours, 45 minutes to 48 hours, 45 minutes to 36 hours, 1-36 hours, 1-24 hours, 2-24 hours, 2-18 hours, 3-18 hours, 3-12 hours, 4-12 hours or 4-6 hours.

In some embodiments, the method:
a) activates the CXCR6-positive leukocyte by increasing the proliferation and/or cytokine production;
b) maintains the survival of the CXCR6-positive leukocyte; or
c) both a) and b).

In some embodiments, the method activates the CXCR6-positive leukocyte. In some embodiments, the method maintains the survival of the CXCR6-positive leukocyte. In some embodiments, the method of activating and/or maintaining the survival of the CXCR6-positive leukocyte comprises contacting the CXCR6-positive leukocyte with a polypeptide, fusion protein or composition of the invention (e.g., an anti-CXCR6 antibody), e.g., at a concentration of about 0.05-20 μg/ml for several minutes up to several days following the usual range of the antibody's half-life in human body. In some embodiments, the method of activating and/or maintaining the survival of the CXCR6-positive leukocyte comprises contacting the CXCR6-positive leukocyte with a polypeptide of the invention (e.g., an anti-CXCR6 antibody) for at least about 1 minute, e.g., at least about: 2 minutes, 3 minutes, 4 minutes, 5 minutes, 6 minutes, 7 minutes, 8 minutes, 9 minutes, 10 minutes, 12 minutes, 15 minutes, 20 minutes, 25 minutes, 30 minutes, 35 minutes, 40 minutes, 45 minutes, 50 minutes, 55 minutes, 60 minutes, 2 hours, 3 hours, 4 hours, 5 hours, 6 hours, 7 hours, 8 hours, 9 hours, 10 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 5 days, 6 days, 7 days, 8 days, 9 days or 10 days. In some embodiments, the method of activating and/or maintaining the survival of the CXCR6-positive leukocyte comprises contacting the CXCR6-positive leukocyte with a polypeptide of the invention (e.g., an anti-CXCR6 antibody) for about 1 minute to 10 days, for example, about: 3 minutes to 10 days, 3 minutes to 8 days, 10 minutes to 8 days, 10 minutes to 6 days, 15 minutes to 6 days, 15 minutes to 4 days, 30 minutes to 4 days, 30 minutes to 48 hours, 45 minutes to 48 hours, 45 minutes to 36 hours, 1-36 hours, 1-24 hours, 2-24 hours, 2-18 hours, 3-18 hours, 3-12 hours, 4-12 hours or 4-6 hours.

In another aspect, the invention provides a method of depleting of CXCR6-positive leukocytes in a subject in need thereof, comprising administering an effective amount of a composition described herein (e.g., a pharmaceutical composition comprising a pharmaceutically acceptable carrier and, as an active ingredient, a polypeptide and/or fusion protein described herein) to the subject.

The term “subject” or “patient” refers to an animal (e.g., a mammal). In some embodiments, the subject is a mammal. In some embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate (e.g., a cynomolgus). In some embodiments, the subject is a human.

In some embodiments, the subject is a pediatric patient. In some embodiments, the subject is a juvenile patient. In some embodiments, the subject is an adult patient.

In some embodiments, the subject is two years of age or older, for example, at least: 3, 4, 5, 6, 7, 8, 9, 10, 11, 12 years of age or older. In some embodiments, the subject is 4 years of age or older. In some embodiments, the subject is 5 years of age or older. In some embodiments, the subject is 6 years of age or older. In some embodiments, the subject is 12 years of age or older. In some embodiments, the subject is 18 years of age or older. In some embodiments, the subject is 18-75 years of age. In some embodiments, the subject is 40 years of age or older, e.g., at least: 45, 50, 55, 60, 65, 70, 75, 80, 85 or 90 years old.

In some embodiments, the subject has or is at risk of developing a disease (e.g., an autoimmune disease). In some embodiments, the subject has or is at risk of developing acute GvHD (aGvHD), chronic GvHD (cGvHD), allograft rejection, asthma, autoimmune hepatitis, autoimmune uveitis, contact dermatitis, Crohn's Disease, juvenile rheumatoid arthritis, multiple sclerosis, nonalcoholic steatohepatitis, psoriasis, psoriatic arthritis, rheumatoid arthritis, systemic lupus erythematosus, uveitis or ulcerative colitis. In some embodiments, the subject has or is at risk of developing aGvHD.

In another aspect, the invention provides a method of treat or preventing a disease (e.g., an autoimmune disease) in a subject in need thereof, comprising administering an effective amount of a composition of the invention (e.g., a pharmaceutical composition described herein) to the subject.

The term “treating” or “treatment” refers to the medical management of a subject with the intent to improve, ameliorate, stabilize (i.e., not worsen), prevent or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease, disorder or condition; preventing spread of the disease, disorder or condition; delay or slowing the progress of the disease, disorder or condition; amelioration or palliation of the disease, disorder or condition; and remission (whether partial or total), whether detectable or undetectable. “Ameliorating” or “palliating” a disease, disorder or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival in the absence of treatment. Those in need of treatment include those already with (e.g., having, comprising, suffering from) the disease, disorder, or condition, as well as those prone to have the condition or disorder or those in which the disease, disorder, or condition is to be prevented.

A subject to be treated according to the methods described herein may be one who has been diagnosed with a particular condition (e.g., an autoimmune disease), or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the disease is aGvHD, cGvHD, allograft rejection, asthma, autoimmune hepatitis, autoimmune uveitis, contact dermatitis, Crohn's Disease, juvenile rheumatoid arthritis, multiple sclerosis, nonalcoholic steatohepatitis, psoriasis, psoriatic arthritis, rheumatoid arthritis, systemic lupus erythematosus, uveitis or ulcerative colitis. In some embodiments, the disease is aGvHD.

In some embodiments, the method is used for prophylactic therapy. In some embodiments, the effective dosage is sufficient to prevent the subject of developing a disease or condition (e.g., an autoimmune disease).

In some embodiments, the polypeptide comprises a heterologous moiety comprising tumor necrosis factor alpha (TNF-α), IL-1B, IL-17, IL-6, IL-12, IL-21, IL-23, IFNγ or GM-CSF, or a combination thereof.

In some embodiments, the method further comprises administering a therapeutically effective amount of at least one additional therapeutic agent to the subject. Non-limiting examples of the additional therapeutic agents include monoclonal antibodies, steroids (e.g., corticosteroids) and immunosuppressants (e.g., agents targeting the IL-23/IL-17 axis). In some embodiments, the polypeptide, fusion protein or composition (e.g., pharmaceutical composition) and the at least one additional therapeutic agent are administered simultaneously. In some embodiments, the composition and the at least one additional therapeutic agent are administered sequentially or separately.

In some embodiments, the additional agent is an antibody, e.g., a monoclonal antibody. In some embodiments, the monoclonal antibody is an anti-CD20 monoclonal antibody (e.g., rituximab), an anti-IL-1β monoclonal antibody (e.g., canakinumab), an anti-IL-6 monoclonal antibody (e.g., clazakizumab, siltuximab or sirukumab) or an anti-IL-6 receptor monoclonal antibody (e.g., olokizumab, sarilumab, satralizumab, tocilizumab or vobarilizumab), an anti-IL-12/IL-23p40 inhibitor (e.g., ustekinumab) or an anti-TNFα monoclonal antibody (e.g., adalimumab, certolizumab pegol or infliximab).

In some embodiments, the steroid is prednisone.

In some embodiments, the immunosuppressant is cyclophosphamide, fingolimod, methotrexate or a Janus kinase (JAK) inhibitor. In some embodiments, the immunosuppressant is cyclophosphamide, fingolimod or methotrexate. In some embodiments, the immunosuppressant is a JAK inhibitor. In some embodiments, the JAK inhibitor is Ruxolitinib (INCB018424), Momelotinib, fedratinib (TG101348) or tofacitinib.

In another aspect, the invention provides a method of treating cancer in a subject in need thereof, comprising administering an effective amount of a polypeptide, fusion protein and/or composition described herein (e.g., a pharmaceutical composition comprising a pharmaceutically acceptable carrier and, as an active ingredient, a polypeptide and/or fusion protein described herein) to the subject.

In some embodiments, the subject is a human patient who has cancer. In some embodiments, the subject is a human patient who is at risk of developing cancer. In some embodiments, the subject is 15 years of age or older.

In some embodiments, the cancer is breast cancer, colon cancer, gastric cancer, liver cancer, lung cancer, lymphoma, melanoma, non-small cell lung cancer or oesophageal cancer (i.e., esophageal cancer). In some embodiments, the cancer is colon cancer.

In some embodiments, the polypeptide comprises a human IgG4 constant region sequence (e.g., SEQ ID NO:346). IgG4 would not kill the tumor-infiltrating T cells via ADCC or CDC.

In some embodiments, the polypeptide is a bispecific antibody that binds an immune checkpoint protein and CXCR6. In some embodiments, the polypeptide is a bispecific antibody that binds PD-1 and CXCR6. In some embodiments, the polypeptide is a bispecific antibody that binds PD-L1 and CXCR6.

In some embodiments, the polypeptide comprises a heterologous moiety comprising IL-2, IL-7 or IL-15, or a combination thereof.

In some embodiments, the composition (e.g., pharmaceutical composition) is administered in combination with a chemotherapeutic agent, a targeted anti-cancer therapy, a standard of care drug for treatment of cancer, or an immune checkpoint inhibitor.

In some embodiments, the immune checkpoint inhibitor is an anti-PD-1 antibody, an anti-PD-L1 antibody, an anti-PD-L2 antibody, an anti-lymphocyte activation gene-3 (LAG-3) antibody, an anti-TIM3 antibody, or an anti-CTLA-4 antibody.

In some embodiments, the immune checkpoint inhibitor is an anti-PD-1 antibody. In some embodiments, the anti-PD-1 antibody is nivolumab, pembrolizumab, spartalizumab, cemiplimab, camrelizumab, tislelizumab, dostarlimab, MEDI-0680 or SSI-361. In some embodiments, the anti-PD-1 antibody is nivolumab. In some embodiments, the anti-PD-1 antibody is pembrolizumab. For additional information on non-limiting examples of anti-PD-1 antibodies, see, e.g., U.S. Pat. No. 7,595,048 (nivolumab), U.S. Pat. No. 8,952,136 (pembrolizumab), U.S. Pat. No. 9,683,048 (spartalizumab), U.S. Pat. No. 9,987,500 (cemiplimab), U.S. Pat. No. 10,344,090 (camrelizumab), U.S. Pat. No. 8,735,553 (tislelizumab), U.S. Pat. No. 9,815,897 (dostarlimab), U.S. Pat. No. 8,609,089 (MEDI-0680) and U.S. Pat. No. 10,913,797 (SSI-361), the contents of which are incorporated herein in their entirety.

In some embodiments, the anti-PD-1 antibody comprises V_{H} and V_{L} amino acid sequences of:
a) SEQ ID NO:349 and SEQ ID NO:350, respectively;
b) SEQ ID NO:351 and SEQ ID NO:352, respectively;
c) SEQ ID NO:353 and SEQ ID NO:354, respectively; or
d) SEQ ID NO:355 and SEQ ID NO:356, respectively.

The sequences identified as SEQ ID NOs:349-356 are shown in Table 9 herein.

In some embodiments, the immune checkpoint inhibitor is an anti-PD-L1 antibody. In some embodiments, the anti-PD-L1 antibody is atezolizumab, avelumab, durvalumab, BMS-936559 or envafolimab. In some embodiments, the anti-PD-L1 antibody is atezolizumab. In some embodiments, the anti-PD-L1 antibody is avelumab. In some embodiments, the anti-PD-L1 antibody is durvalumab. For additional information on non-limiting examples of anti-PD-L1 antibodies, see, e.g., U.S. Pat. No. 8,217,149 (atezolizumab), U.S. Pat. No. 9,624,298 (avelumab) and U.S. Pat. No. 8,779,108 (durvalumab), U.S. Pat. No. 7,943,743 (BMS-936559) and United States Patent Publication No: US20180327494 (envafolimab), the contents of which are incorporated herein in their entirety.

In some embodiments, the anti-PD-L1 antibody comprises V_{H} and V_{L} amino acid sequences of:
a) SEQ ID NO:357 and SEQ ID NO:358, respectively;
b) SEQ ID NO:359 and SEQ ID NO:360, respectively; or
c) SEQ ID NO:361 and SEQ ID NO:362, respectively.

The sequences identified as SEQ ID NOs:357-362 are shown in Table 9 herein.

In some embodiments, the immune checkpoint inhibitor is an anti-PD-L2 antibody.

In some embodiments, the immune checkpoint inhibitor is an anti-LAG-3 antibody. In some embodiments, the anti-LAG-3 antibody is relatlimab, BMS-986016 or REGN3767. Also see, e.g., U.S. Pat. No. 10,344,089, the contents of which are incorporated herein in their entirety.

In some embodiments, the immune checkpoint inhibitor is an anti-TIM-3 antibody. In some embodiments, the anti-TIM-3 antibody is MGB453, TSR-022, Sym023, BGBA425, R07121661, LY3321367, ICAGN02390 or BMS-986258. For additional information on anti-TIM-3 antibodies, see, e.g., Acharya et al., Tim-3 finds its place in the cancer immunotherapy landscape, J Immunother Cancer 8(1):e000911 (2020), the contents of which are incorporated herein in their entirety.

In some embodiments, the anti-TIM-3 antibody comprises V_{H} and V_{L} amino acid sequences of:
a) SEQ ID NO:363 and SEQ ID NO:364, respectively; or
b) SEQ ID NO:365 and SEQ ID NO:366, respectively.

The sequences identified as SEQ ID NOs:363-366 are shown in Table 9 herein.

In some embodiments, the immune checkpoint inhibitor is an anti-CTLA-4 antibody. In some embodiments, the anti-CTLA-4 antibody is ipilimumab.

The anti-PD-1, anti-PD-L1, anti-PD-L2, anti-LAG3, anti-TIM3 and anti-CTLA-4 antibodies may be generated de novo.

In some embodiments, the immune checkpoint inhibitor is a probody (e.g., CX-072 (pacmilimab)).

In some embodiments, the immune checkpoint inhibitor is a recombinant fusion protein (e.g., AMP-224).

In some embodiments, the polypeptide, fusion protein or composition (e.g., pharmaceutical composition) and the immune checkpoint inhibitor are administered simultaneously. In some embodiments, the polypeptide, fusion protein or composition and the immune checkpoint inhibitor are administered sequentially or separately.

In some embodiments, a method of the invention further comprises administering radiation therapy, chemotherapy, surgery or a combination thereof. Non-limiting examples of radiation therapies include external beam radiation, intensity modulated radiation therapy (IMRT), focused radiation, and any form of radiosurgery including GAMMA KNIFE®, CYBERKNIFE®, linear accelerator (Linac), and interstitial radiation (e.g., implanted radioactive seeds, GliaSite balloon).

Focused radiation methods that may be used include stereotactic radiosurgery, fractionated stereotactic radiosurgery, and intensity-modulated radiation therapy (IMRT). It is apparent that stereotactic radiosurgery involves the precise delivery of radiation to a tumorous tissue, for example, a brain tumor, while avoiding the surrounding nontumorous, normal tissue. The dosage of radiation applied using stereotactic radiosurgery may vary, typically from 1 Gy to about 30 Gy, and may encompass intermediate ranges including, for example, from 1 to 5, 10, 15, 20, 25, up to 30 Gy in dose. Because of noninvasive fixation devices, stereotactic radiation need not be delivered in a single treatment. The treatment plan may be reliably duplicated day-to-day, thereby allowing multiple fractionated doses of radiation to be delivered. When used to treat a tumor over time, the radiosurgery is referred to as “fractionated stereotactic radiosurgery” or FSR. In contrast, stereotactic radiosurgery refers to a one-session treatment. Fractionated stereotactic radiosurgery may result in a high therapeutic ratio, i.e., a high rate of killing of tumor cells and a low effect on normal tissue. The tumor and the normal tissue respond differently to high single doses of radiation vs. multiple smaller doses of radiation. Single large doses of radiation may kill more normal tissue than several smaller doses of radiation may. Accordingly, multiple smaller doses of radiation can kill more tumor cells while sparing normal tissue. The dosage of radiation applied using fractionated stereotactic radiation may vary from range from 1 Gy to about 50 Gy, and may encompass intermediate ranges including, for example, from 1 to 5, 10, 15, 20, 25, 30, 40, up to 50 Gy in hypofractionated doses. Intensity-modulated radiation therapy (IMRT) may also be used. IMRT is an advanced mode of high-precision three-dimensional conformal radiation therapy (3DCRT), which uses computer-controlled linear accelerators to deliver precise radiation doses to a malignant tumor or specific areas within the tumor. In 3DCRT, the profile of each radiation beam is shaped to fit the profile of the target from a beam's eye view (BEV) using a multileaf collimator (MLC), thereby producing a number of beams. IMRT allows the radiation dose to conform more precisely to the three-dimensional (3-D) shape of the tumor by modulating the intensity of the radiation beam in multiple small volumes. Accordingly, IMRT allows higher radiation doses to be focused to regions within the tumor while minimizing the dose to surrounding normal critical structures. IMRT improves the ability to conform the treatment volume to concave tumor shapes, for example, when the tumor is wrapped around a vulnerable structure, such as the spinal cord or a major organ or blood vessel.

### Therapeutic Administration

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent (e.g., compound or composition described herein) to be administered can be determined by a clinician of ordinary skill using the guidance provided herein and other methods known in the art. Relevant factors include the given agent, the pharmaceutical formulation, the route of administration, the type of disease or disorder, the identity of the subject (e.g., age, sex, weight) or host being treated, and the like. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments it is an amount of the composition sufficient to achieve a treatment response as compared to the response obtained without administration of the composition. In other embodiments, it is an amount that results in a beneficial or desired result in a subject as compared to a control. As defined herein, a therapeutically effective amount of a composition of the present disclosure may be readily determined by one of ordinary skill by routine methods known in the art. Dosage regimen and route of administration may be adjusted to provide the optimum therapeutic response.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. The carrier may be diluent, adjuvant, excipient, or vehicle with which the polypeptide is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the polypeptide antibody in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

The mode of administration, e.g., of the polypeptides, fusion proteins, polynucleotides, expression vectors or host cells or compositions (e.g., pharmaceutical compositions) may be any suitable parenteral or nonparenteral administration, including those described herein. Non-limiting examples of administration routes include oral, nasal, vaginal, rectal, mucosal, intravenous, intramuscular, subcutaneous, intradermal, intraperitoneal, pulmonary, topical and transmucosal (oral, intranasal, intravaginal or rectal). In some embodiments, the composition is formulated for intravenous administration.

In some embodiments, the composition (e.g., pharmaceutical composition) is administered by intravenous infusion. In some embodiments, the intravenous infusion is given over 15, 30, 45 or 60 minutes. In some embodiments, the intravenous infusion is given over 1.5, 2, 3, 4, 5, 6, 7, 8, 9, 10, 11 or 12 hours.

The dose, e.g., the dose of a composition, polynucleotide, fusion protein or polypeptide given to a subject (e.g., a human patient) is sufficient to alleviate or at least partially arrest the disease being treated (“therapeutically effective amount”). Non-limiting examples of therapeutically effective amounts include about 0.005 mg to about 100 mg/kg, e.g. about: 0.05-30, 5-25, 1, 2, 3, 4, 5, 6, 7, 8, 9, 10, 15, 16, 17, 18, 19, 20, 21, 22, 23, 24, 25, 30, 40, 50, 60, 70, 80, 90 or 100 mg/kg. In some embodiments, the dose is based on the patient's surface area, e.g., 500, 400, 300, 250, 200, or 100 mg/m². A fixed unit dose may also be given, for example, as an initial dose of at least about 0.1 mg, for example, at least about: 1, 5, 10, 50, 100, 200, 500 or 1000 mg; about: 0.1-800 mg, 1-600 mg, 5-500 mg, or 10-400 mg.

The dosage may also depend on the disease. Usually between 1 and 8 doses, e.g., 1, 2, 3, 4, 5, 6, 7 or 8, may be administered. In some embodiments, 9, 10, 11, 12, 13, 14, 15, 16, 17, 18, 19, 20 or more doses may be administered.

The administration, e.g., administration of the polypeptide, fusion protein or composition (e.g., pharmaceutical composition) may be repeated. In some embodiments, the initial dose is followed by administration of a second or a plurality of subsequent doses of the composition (e.g., pharmaceutical composition), for example, wherein the subsequent doses are separated by at least 1-3 days, e.g., at least: 1, 2, 3, 4, 5 or 6 days, 1, 2, 3, 4, 5, 6 or 7 weeks, or 1, 2, 3, 4, 5 or 6 months, or longer.

In some embodiments, the repeated administration is in an amount that is approximately the same or less than that of the initial dose. In some embodiments, the repeated administration is in an amount that is at a higher dose. For example, the polypeptide (e.g., antibody) may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. In some embodiments, the administration is in cycles, e.g., weekly, biweekly, 28-day or monthly. In some embodiments, the subject had previously received or been administered a therapy described herein. In other embodiments, the subject is treatment naïve.

### Method of Detection

In another aspect, the invention provides a method of detecting and/or quantifying CXCR6, comprising contacting a sample (e.g., a biological sample from a subject) with a polypeptide, a fusion protein, or a composition described herein and determining whether CXCR6 is present in the sample and/or measuring the level of CXCR6 in the sample. In some embodiments, the method is used for diagnosing or prognosing a CXCR6-associated disease or disorder (e.g., an autoimmune disease) in a subject.

In some embodiments, the polypeptide is conjugated with a detectable label or reporter molecule (e.g., at or near its N- or C-terminus). Alternatively, an unlabeled polypeptide of the invention (e.g., an anti-CXCR6 antibody) is used in combination with a detectably labeled secondary antibody. Non-limiting examples of detectable labels or reporter molecules include a biotin, a radioisotope (e.g., ³H, ¹⁴C, ³²P, ³⁵S, or ¹²⁵I), a fluorescent or chemiluminescent moiety (e.g., fluorescein isothiocyanate, or rhodamine), and an enzyme (e.g., alkaline phosphatase, β-galactosidase, horseradish peroxidase, or luciferase).

Non-limiting examples of suitable assays include enzyme-linked immunosorbent assay (ELISA), fluorescence-activated cell sorting (FACS) and radioimmunoassay (RIA).

Non-limiting examples of biological samples include any tissue or fluid sample obtainable from a subject (e.g., human patient) that contains a detectable quantity of CXCR6 protein or fragment(s) thereof, under normal or pathological conditions.

In some embodiments, the level of CXCR6 in the biological sample is compared to a baseline, or standard, level of CXCR6. In some embodiments, a level of CXCR6 in a biological sample obtained from a subject not afflicted with a disease associated with CXCR6 (e.g., a healthy subject) is used as a baseline, or standard, level of CXCR6.

### Exemplification

The principle behind the therapy for autoimmune disorders is to selectively delete or compromise the pathogenic T cells without affecting the non-pathogenic T cells, thereby minimizing side effects and preserving protective T cell functions. The principle for tumor treatment is to selectively expand and/or activate tumor-infiltrated T cells while leaving other T cell populations minimally manipulated to avoid the side effects. As the consequence, the major challenge in both cases is to find a marker that can be used to specifically identify the pathogenic T cells in immune disorders and the immune competent T cells infiltrated in the tumor.

CXCR6, also named CD186 or Bonzo or STRL33, belongs to the family of G-protein-coupled receptors commonly called GPCRs. CXCR6 is a chemokine receptor initially found to be expressed by a subset of activated T cells, γδ T cells, NK cells and NK T cells. CXCR6 positive T cells have been involved in many immune disorders including psoriasis, allograft rejection, multiple sclerosis, Crohn's disease, pulmonary infection with Francisella tularensis live vaccine strain, etc. However, genetic deficiency of CXCR6 in murine models only marginally ameliorates or exerts no effects on immunopathology of all of the above-mentioned immune disorders. Thus, altering expression of CXCR6 may not be effective in treating inflammation and immune disorders.

The significance of CXCR6 was defined as an exquisite marker of pathogenic T cells in multiple sclerosis, which have the properties of: rapidly proliferating; highly producing proinflammatory cytokine IL-17, GM-CSF and IFNγ; highly enriched in the inflamed tissue; containing cytotoxic granules. Depletion of this population by anti-CXCR6 monoclonal antibody (mAb) dramatically reverted the animal model of multiple sclerosis, confirming that CXCR6 marks the pathogenic CD4 cells. As disclosed herein, CXCR6 is highly expressed by most pathogenic CD4 and/or CD8 T cells in collagen-induced arthritis and acute graft versus host disease (aGvHD). Moreover, while a deficiency of CXCR6 molecule in grafted immune cells did not ameliorate aGvHD, administration of an anti-CXCR6 mAb largely improved aGvHD disease, indicating that CXCR6 expression identifies the pathogenic cells in aGvHD.

CXCR6 is further defined herein as a specific marker of the immune-competent T cells infiltrated in the tumor. CXCR6 is expressed primarily by tumor-infiltrated CD4 and CD8 T cells, particularly the immune competent cells with higher IFN-γ and GM-CSF production among CD4 T cells, and higher granzyme-B expression among CD8 T cells. Because CXCR6⁺ T cells are only a minor population within the peripheral immune organ, their activation is unlikely to induce systemic immune activation or severe side effects.

Of note, although CXCR6 is dispensable for the function of CD4 and CD8 T cells in these disorders, CXCR6 was reported to be essential for a subset of NK T cells to mediate the hepatic inflammation and fibrogenesis. Also, CXCR6-positive CD8 T cells were reported to mediate the nonalcoholic steatohepatitis (NASH) through an auto-aggressive activation mechanism.

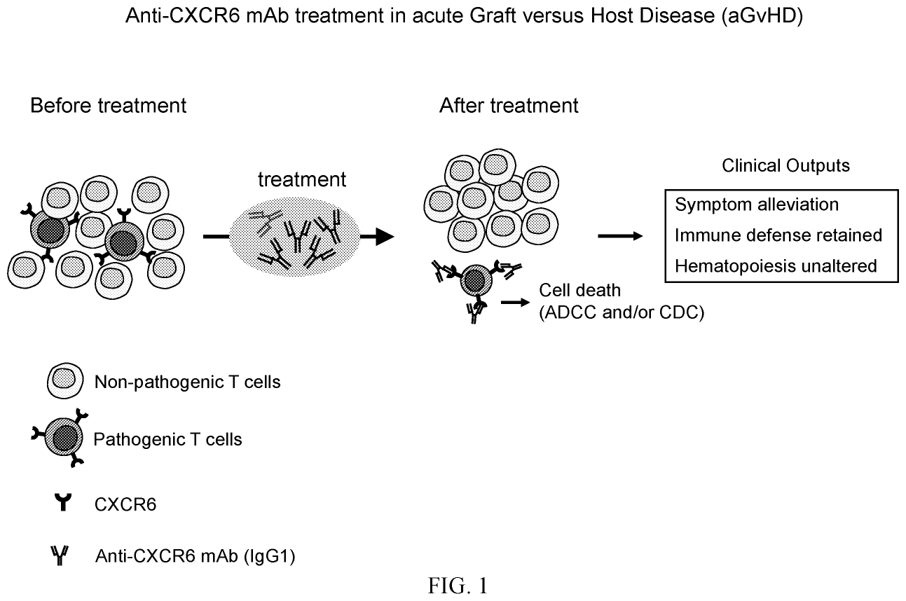

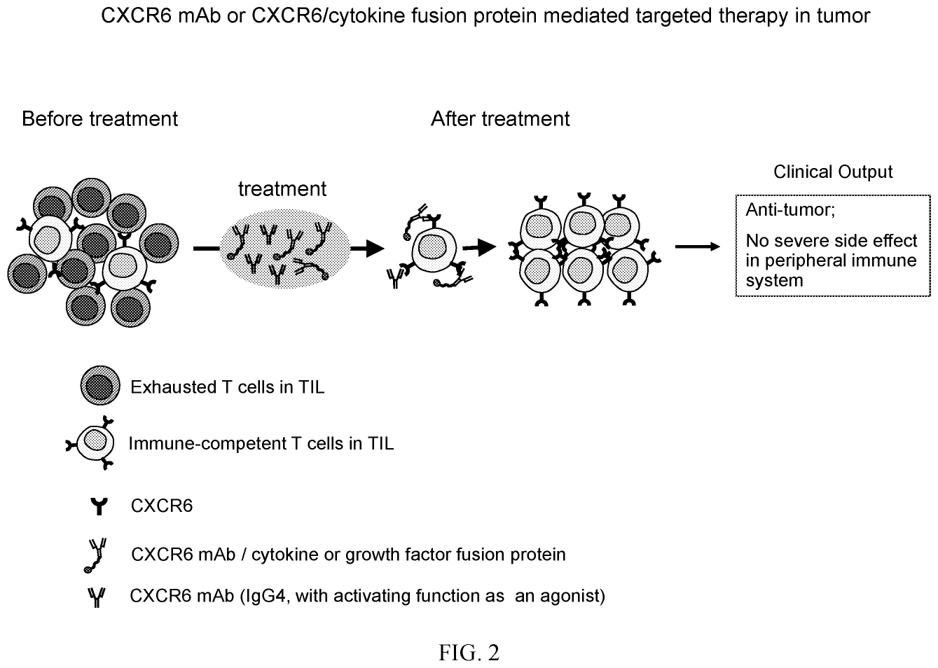

Thus, novel agents such as novel peptides (e.g., antibodies or antigen-binding fragments thereof) that specifically bind CXCR6 are needed. For example, anti-CXCR6 antibodies that specifically bind both CXCR6 of human origin and CXCR6 of a non-human mammalian origin may be useful as therapeutics and for preclinical and clinical in vivo studies. Specific, high affinity anti-CXCR6 antibodies may be needed for blocking CXCR6 interaction with its natural ligand, CXCL-16. Anti-CXCR6 antibodies inducing ADCC, ADCP and/or CDC may be needed for depleting of the CXCR6-positive pathogenic T cells in diseases such as autoimmune diseases, NASH, aGvHD, allograft rejection, acute viral infection or T cell-driven cytokine storm (FIG. 1). Anti-CXCR6 antibodies with or without inducing ADCC, ADCP, and CDC, may also be needed for activating tumor-infiltrated CXCR6-positive T cells to treat a tumor, e.g., via direct activation or a conjugated growth factor (FIG. 2).

### Example 1. CXCR6 Identifies Pathogenic CD4 T Cells in Collagen-Induced Arthritis

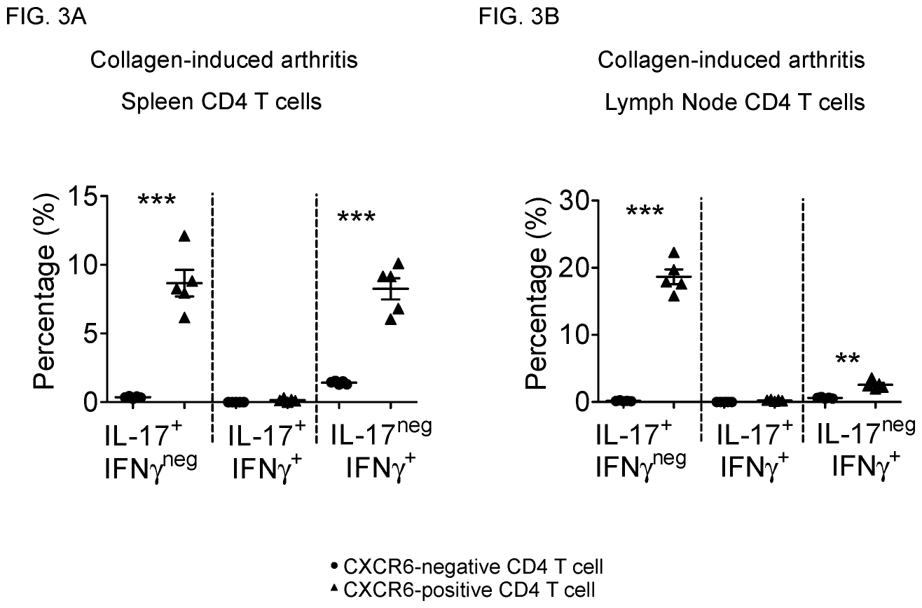

The experiments illustrated in FIGS. 3A-3B demonstrate that CXCR6 identifies pathogenic CD4 T cells in collagen-induced arthritis. Collagen-induced arthritis was induced in male DBA/1 mice by immunization with an emulsion of complete Freund's adjuvant and type II collagen on day 1 and boosting with the same emulsion on day 21. Following the boost, almost 100% of the mice showed arthritis symptoms similar to human rheumatoid arthritis by day 29. In this model, autoreactive CD4 T cells are the driving force for arthritis. Mice were sacrificed on day 35, and both spleen (FIG. 3A) and lymph node (FIG. 3B) were subjected to immunological evaluation. Cytokine profiles in CXCR6-negative and CXCR6-positive CD4 T cells suggest that CXCR6-positive CD4 T cells are the major population producing pro-inflammatory cytokine IL-17 and IFNγ.

### Example 2. CXCR6-Positive T Cells are the Major Pathogenic T Cells in aGvHD

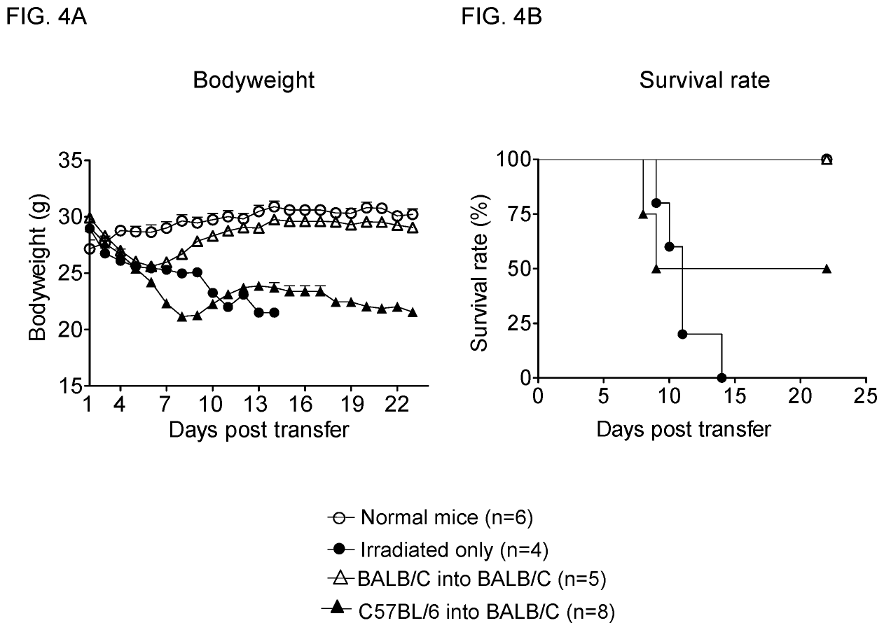

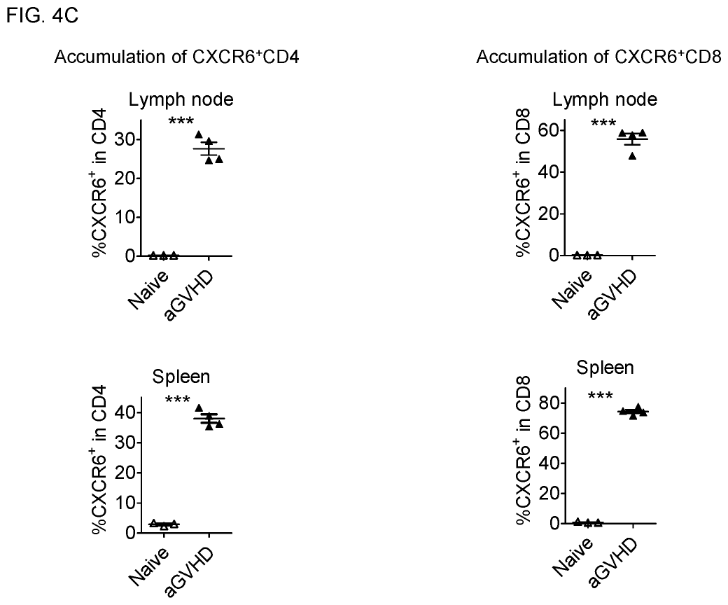

The experiments illustrated in FIGS. 4A-4C demonstrate that CXCR6-positive T cells are the major pathogenic T cells in acute graft versus host disease (aGvHD). aGvHD was induced by transferring bone marrow cells and splenocytes derived from C57BL/6 mice (donors) into lethally irradiated BALB/C mice (recipients). One group of irradiated recipients, identified as “irradiated only,” did not receive cell transfer and died by day 14. Another group of irradiated recipients, identified as “BALB/C into BALB/C,” received bone marrow and splenocytes from the same strain and survived normally. The recipient BALB/C mice that received C57BL/6-derived bone marrow cells and splenocytes, identified as “C57BL/6 into BALB/C,” developed aGvHD disease manifested as decreased body weight (FIG. 4A) and early death (FIG. 4B). On day 6 post transfer, significant accumulations of CXCR6-positive CD4 and CD8 T cells were observed in the spleen and lymph node in the recipient mice (FIG. 4C), which were not detected in naive BALB/C mice.

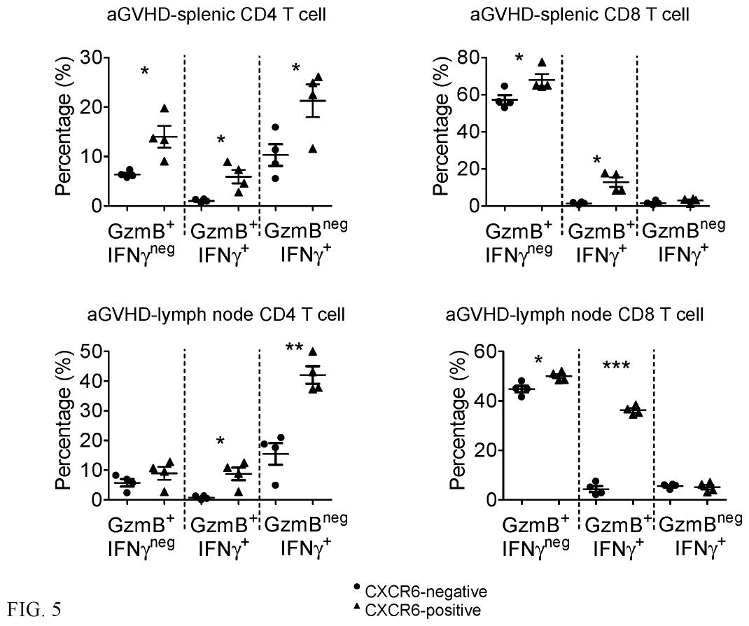

CXCR6-positive CD4 and CD8 T cells were highly pathogenic, producing significantly increased cytokines compared to CXCR6-negative cells. In particular, the IFNγ and GzmB double positive cells, a highly proinflammatory subpopulation, were exclusively CXCR6 positive in both CD4 and CD8 cells (FIG. 5).

### Example 3. CXCR6 is Dispensable in aGvHD Development while Depleting CXCR6-Positive T Cells Ameliorates aGvHD

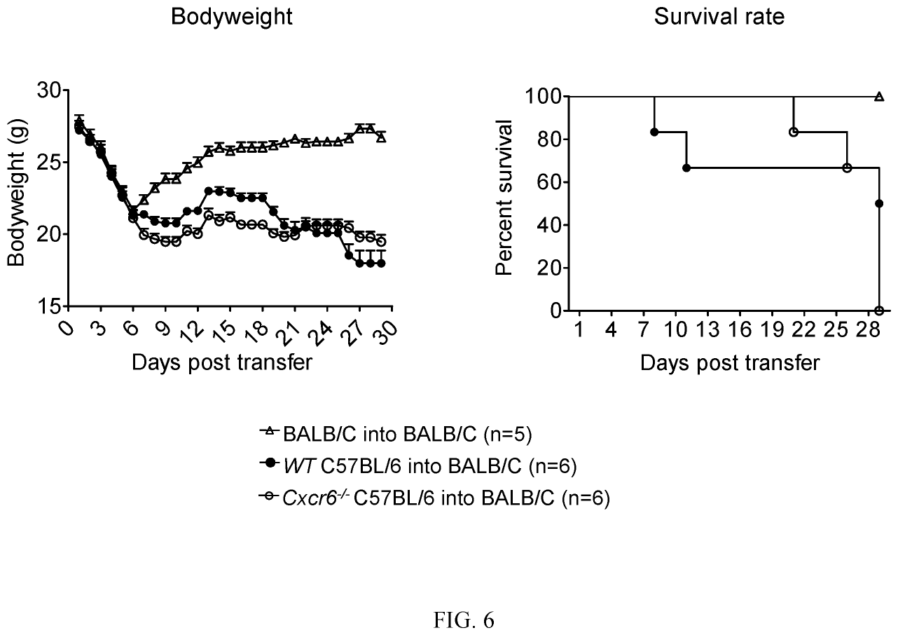

The experiments illustrated in FIG. 6 demonstrate that CXCR6 is dispensable in aGvHD development. BALB/C mice were lethally irradiated and transferred with bone marrow and splenocytes from WT C57BL/6 or Cxcr6^{-/-} C57BL/6 mice. The BALB/C mice that received either WT C57BL/6 or Cxcr6^{-/-} C57BL/6 donor cells developed equally severe aGvHD, as evidenced by their similarly substantial and prolonged decreased bodyweight in both groups; and deficiency of Cxcr6 on donor cells didn't increase the survival (FIG. 6). In contrast, mice from the control group, transferred with cells from BALB/C donors, recovered well from an initial bodyweight loss and all survived (FIG. 6).

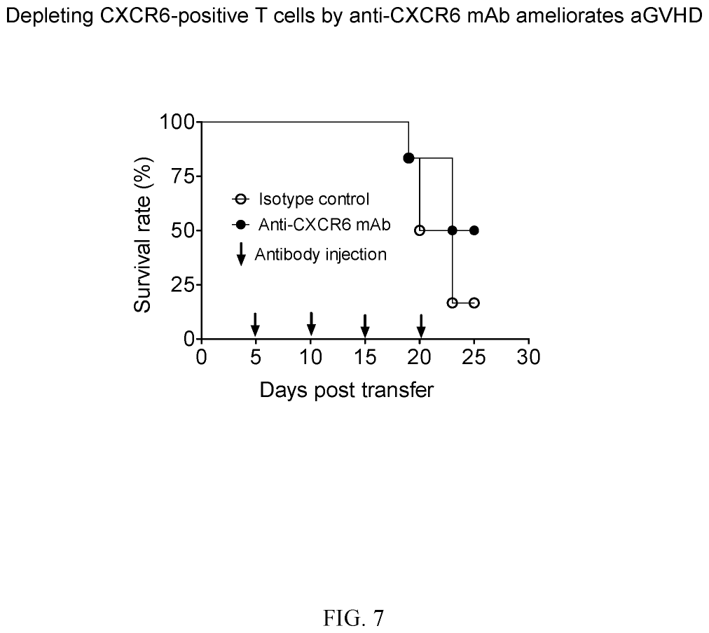

A different experiment, illustrated in FIG. 7, demonstrates that CXCR6 may only serve as the marker for pathogenic T cells in aGvHD, and anti-CXCR6 antibody treatment ameliorates the aGvHD disease by improving the survival.

### Example 4. CXCR6⁺ T Cells in B16 Melanoma and MC38 Colon Tumor Models

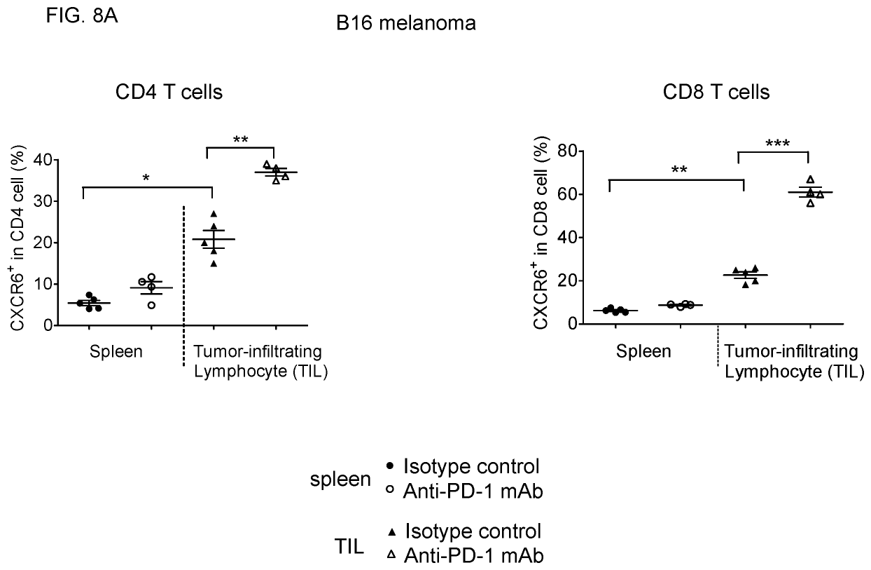

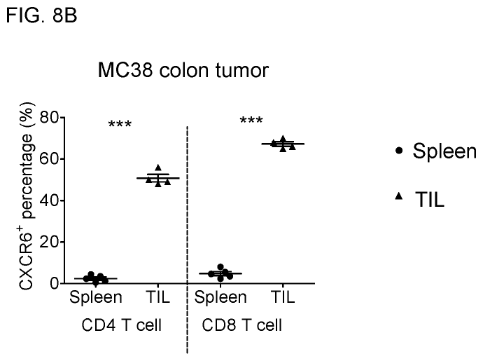

The experiments illustrated in FIGS. 8A-10B demonstrate that CXCR6-positive T cells are enriched in tumor-infiltrating lymphocytes (TIL). B16 is a “cold tumor” that does not trigger a robust immune response. In contrast, MC38 does trigger a robust immune response. To generate the B16 melanoma model, wild type CB7BL/6 mice were inoculated with B16 melanoma and treated with isotype control mAb or anti-PD-1 mAb every 4 days. Mice were sacrificed on day 20, and splenocytes and TIL were analyzed by flow cytometry. To generate the MC38 colon tumor model, wild type CB7BL/6 mice were inoculated with MC38 colon tumor cells and sacrificed on day 20. Splenocytes and TIL were analyzed by flow cytometry. CXCR6⁺ CD4 and CD8 T cells were enriched in TIL in both B16 melanoma model and MC38 colon tumor model (FIGS. 8A-8B). CXCR6⁺ T cells were preferentially accumulated in TIL (FIGS. 8A-8B) and could be expanded by PD-1 antibody treatment (FIG. 8A).

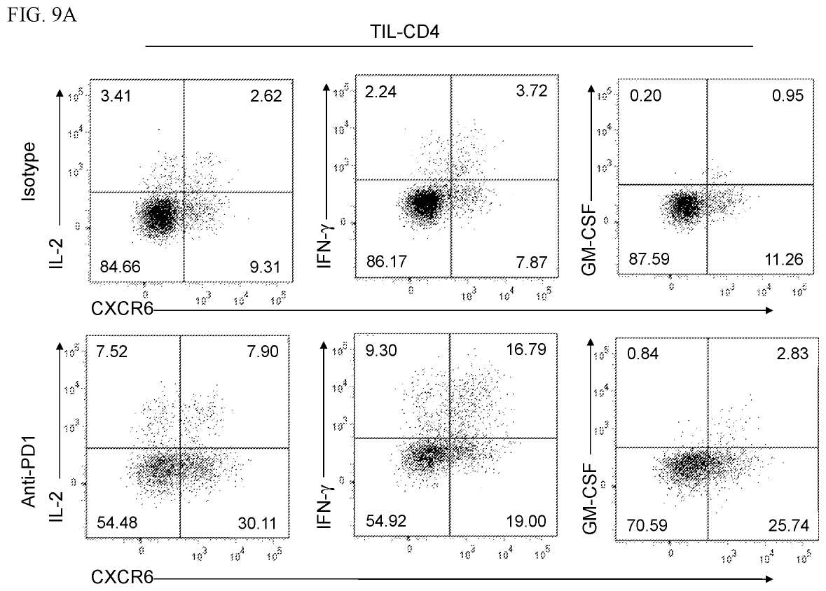

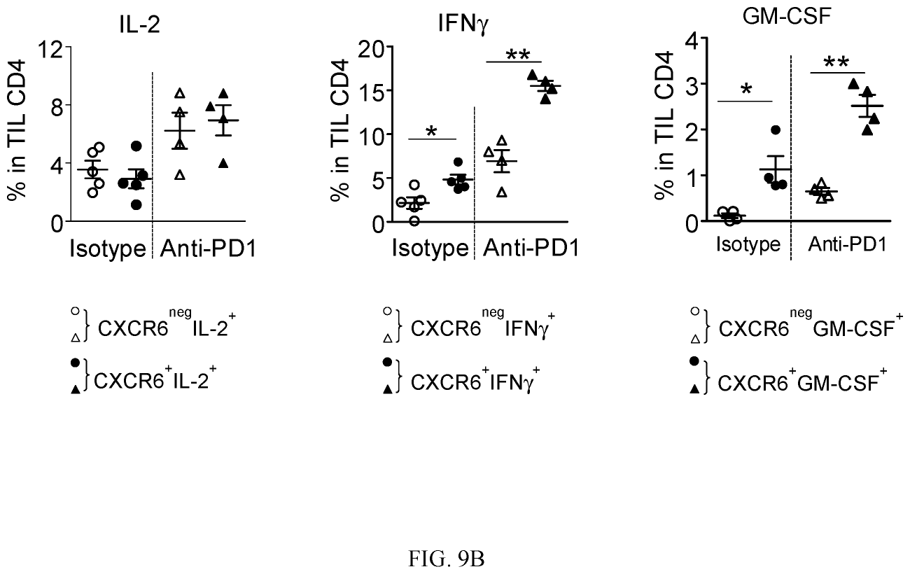

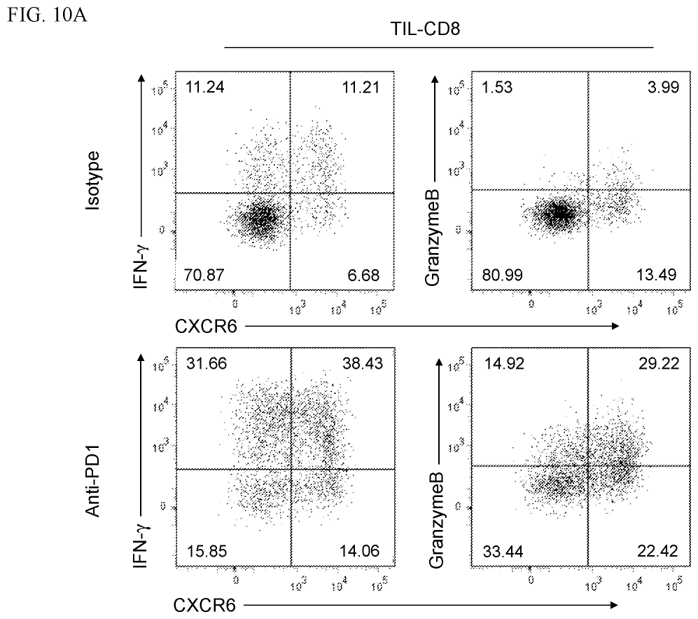

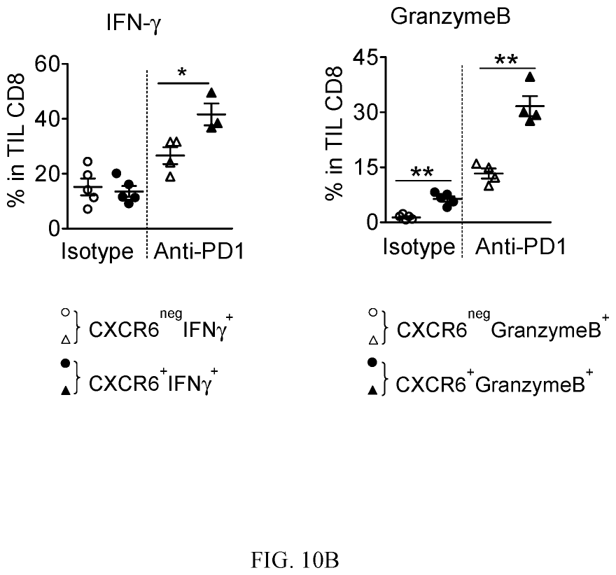

In the B16 melanoma model, mice were sacrificed on day 20, and TIL were stimulated with phorbol myristate acetate (PMA)/ionomycin for four hours in the presence of Brefeldin A (BFA) (10 μg/ml) before analyzed by flow cytometry for cytokine expression. As shown in FIGS. 9A-9B, CXCR6-positive CD4 T cells are the major IFN-γ and GM-CSF producers in TIL, suggesting that they are the major effector cells in TIL. As shown in FIGS. 10A-10B, CXCR6-positive CD8 T cells are the major Granzyme B producers in TIL, suggesting that they are the major killer cells in TIL.

### Example 5. Antibody Production and Characterization

Wild type C57BL/6 mice were immunized and boosted with a full-length human CXCR6-vector. Prior to immunization, the vector was tested for cell surface expression of native human CXCR6 on transfected mammalian cell lines by flow cytometry to ensure a successful immunization and screening. Genetic immunization was performed by ballistically delivering DNA-coated gold particles into the skin of wild type C57BL/6 mice to elicit a target-specific immune response. After the serum test showing satisfactory titer of antibody binding to both human CXCR6 and cyno monkey CXCR6, mice were sacrificed. A single B cell platform was used to sequentially screen bone marrow antibody-producing plasma cells for binding to both human and cyno monkey CXCR6. The conventional hybridoma technique was used on splenocytes and lymph node cells. Candidate clones that specifically bind to human and/or cyno monkey CXCR6 over-expressing cells but not parental cells were identified. After sequencing the V_{H} and V_{L}, clones with distinct CDRs were further expressed as full-length murine antibodies or human chimeric antibodies. The abilities of the individual antibodies to bind to cells overexpressing human-CXCR6 (hu-CXCR6), cyno monkey CXCR6 (cyno-CXCR6) or mouse CXCR6 were tested.

Cells overexpressing human-CXCR6 (hu-CXCR6), cyno monkey CXCR6 (cyno-CXCR6) or mouse CXCR6 were cultured in DMEM medium containing 10% FCS. Before using, cells were collected, washed, and re-suspended in ice-cold phosphate-buffered saline (PBS) contained 0.5% Bovine serum albumin (BSA), and stained with antibodies with serial dilutions. After two rounds of washing with PBS/0.5% BSA buffer, cells were further stained with fluorescence dye-labeled secondary antibody. After two more rounds of washing, cells were resuspended in PBS/0.5% BSA, fixed, and collected by flow cytometry and analyzed.

All of the antibodies in Tables 2 and 3 were tested for binding to human or cynomonkey CXCR6 or blank cell line at the screening stage (with one single concentration to get “yes” or “no”). SEQ ID NOs: 11-17 are human/cyno cross species binders; all the rest (SEQ ID NOs: 18-46) are human-only binders.

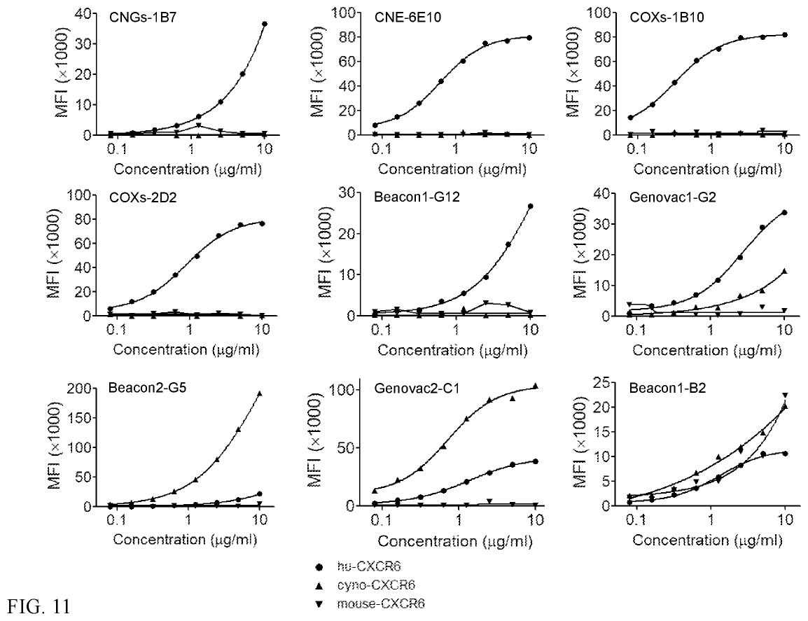

FIG. 11 is the further analysis of the top hits with various concentration of antibodies. Based on the initial screening data, 5 top hits of human-only binders were chosen (CNGs-1B7, CNE-6E10, COXs-1B10, COXs-2D2 and Beacon1-G12), and 3 of them (CNE-6E10, COXs-1B10, and COXs-2D2) were found to have higher affinity than the other two clones (CNGs-1B7 and Beacon1-G12), which can be concluded from FIG. 11. Then, 3 human only-binders (CNE-6E10, COXs-1B10, and COXs-2D2) were chosen for further characterization.

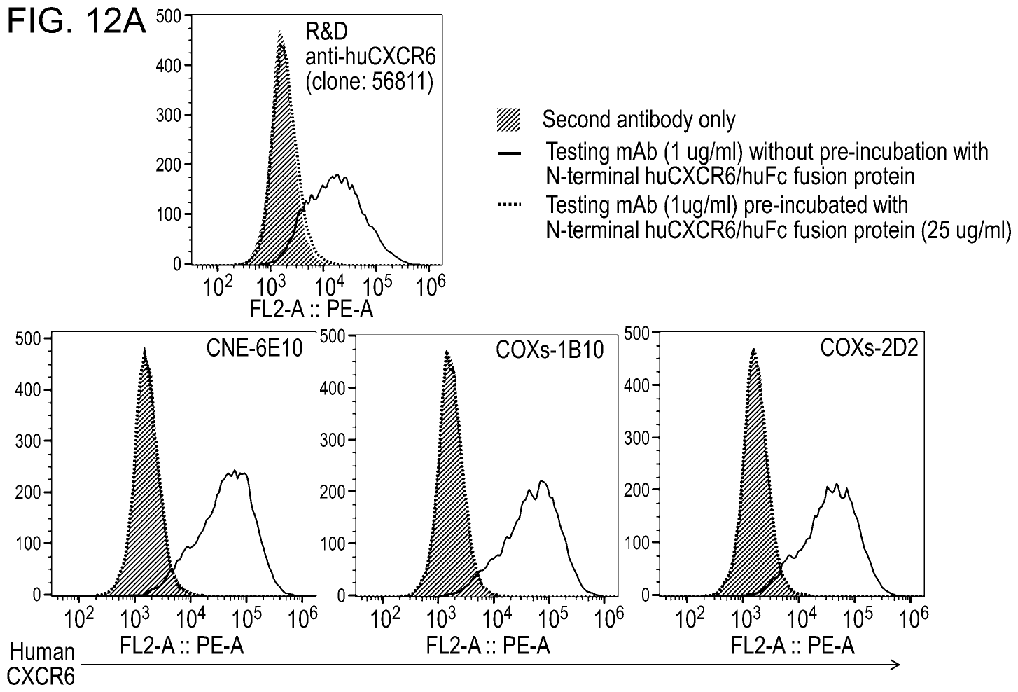

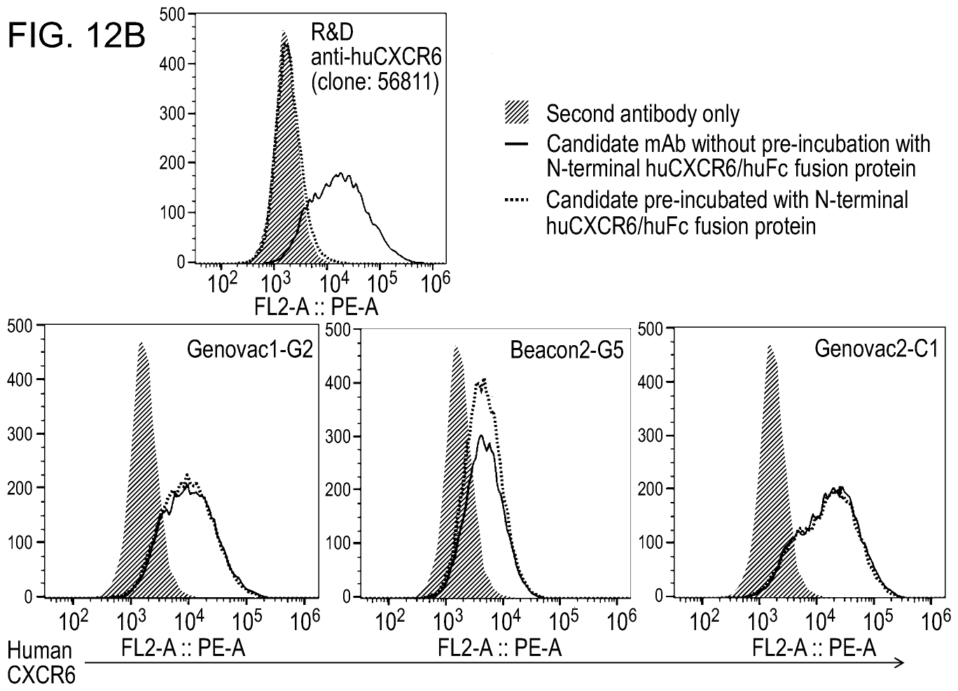

Regarding human/cyno monkey cross-species binders, 3 out of 4 tested clones in FIG. 11 were carried forward for further characterization (FIG. 12). Beacon1-B2 was not further characterized in the experiment illustrated in FIG. 12, since that antibody binds to all of human, cyno monkey, and mouse CXCR6, which is unique. In addition, Beacon1-B2's affinity to human CXCR6 is not as excellent as the other 3 human/cyno monkey cross-species binders.

The experiments illustrated in FIGS. 12A-14 demonstrate that human-specific monoclonal antibodies and human/cyno-monkey cross-species monoclonal antibodies bind to different epitope residues of CXCR6. Three candidate antibodies that bind only to human CXCR6 and three candidate antibodies that bind to both human and cyno CXCR6 were further analyzed to locate their epitope residues. An N-terminal fusion protein was generated by linking the extracellular N terminus of human CXCR6 and the Fc region of human IgG1. Prior to staining cells over-expressing human CXCR6, the candidate antibodies (1 μg/ml) were pre-incubated with the N-terminal fusion protein (25 μg/ml) for 15 minutes at room temperature. A commercial antibody (R&D system, Minneapolis, MN) that only binds to human CXCR6 but not cyno- or mouse-CXCR6, was used as a control antibody. As shown in FIG. 12A, the human-only binders bind to the extracellular N-terminus of human CXCR6. As shown in FIG. 12B, the human/cyno-dual binders do not bind to the extracellular N-terminus of human CXCR6. The results suggest that the human/cyno-dual binders bind to one or more extracellular loops of human CXCR6.

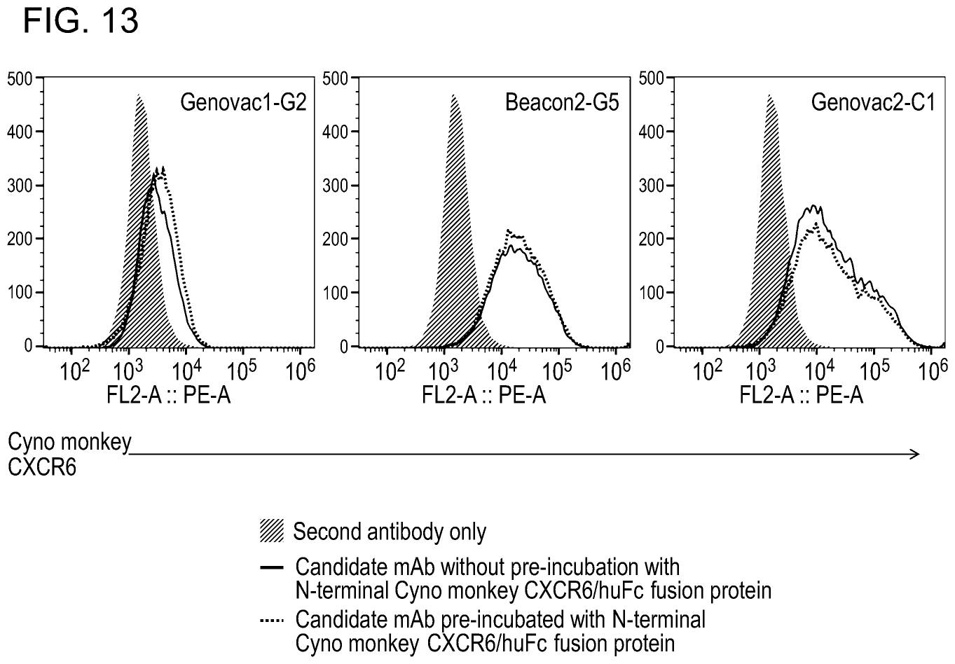

To independently test whether these three human/cyno-dual binders bind to the extracellular N terminus of cyno-CXCR6, a fusion protein linking the extracellular N terminus of cyno monkey CXCR6 and the Fc region of human IgG1 was generated. Prior to staining cells over-expressing cyno monkey CXCR6, the antibodies (1 μg/ml) were pre-incubated with the fusion protein (25 μg/ml) for 15 min at room temperature. As shown in FIG. 13, the human/cyno-dual binders also do not bind to the extracellular N terminus of cyno-CXCR6, suggesting that they likely bind to one or more extracellular loops of cyno-CXCR6.

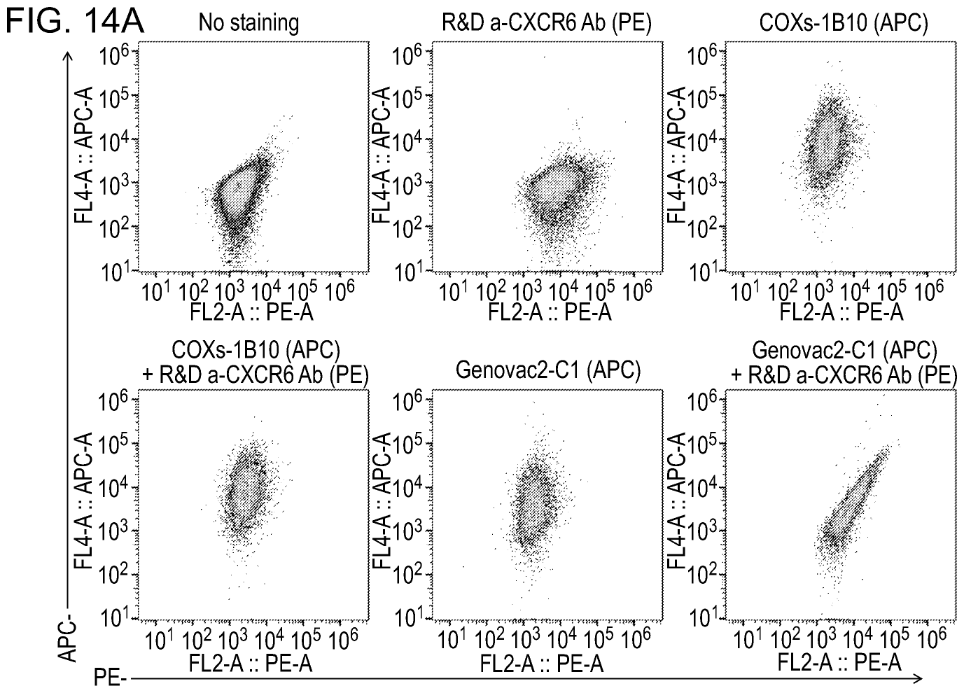

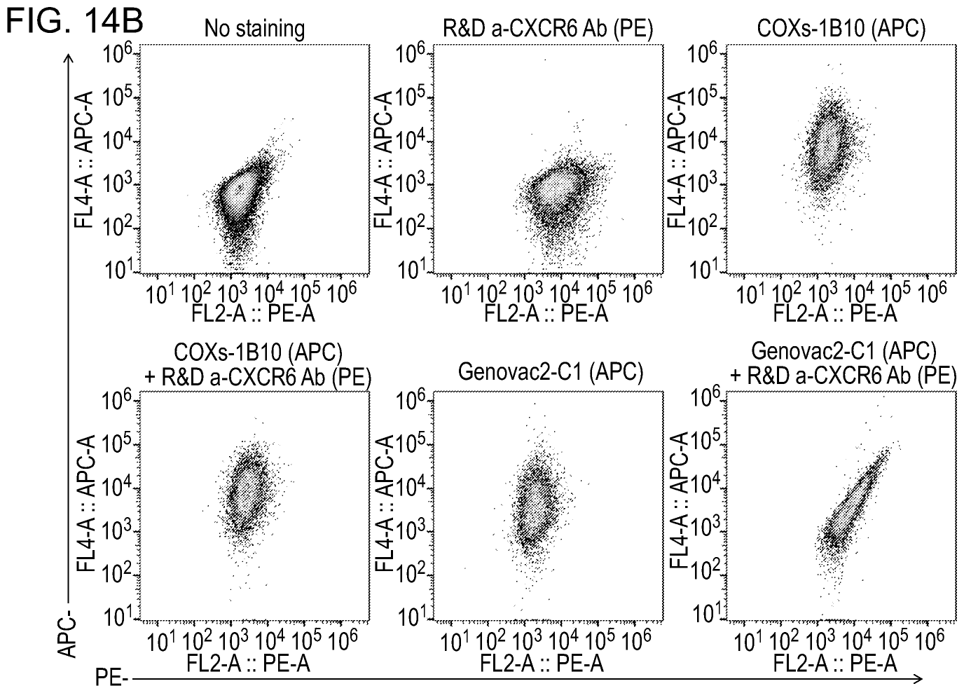

These conclusions are consistent with results from the antibody competition experiments (FIGS. 14A-14B). Human CXCR6 over-expressing cells were pre-incubated with either COXs-1B10 (a human-only binder) or Genovac2-C1 (a human/cyno monkey cross-species binder) for 15 min, followed by staining with APC-labeled anti-mouse IgG (secondary antibody). The cells were washed and stained with a phycoerythrin (PE)-labeled, human-only binder (R&D system, Minneapolis, MN). As shown in FIGS. 14A-14B, only the human/cyno monkey cross-species binder (Genovac2-C1), but not the human-only binder (COXs-1B10), could co-stain cells over-expressing human CXCR6 with the commercial human-only binder (R&D system, Minneapolis, MN). Thus, Genovac2-C1 binds to one or more extracellular loops of human CXCR6, while COXs-1B10 binds to the extracellular N-terminus of human CXCR6.

### Example 6. COXs-1B10 and Genovac2-C1 Chimeric Antibodies

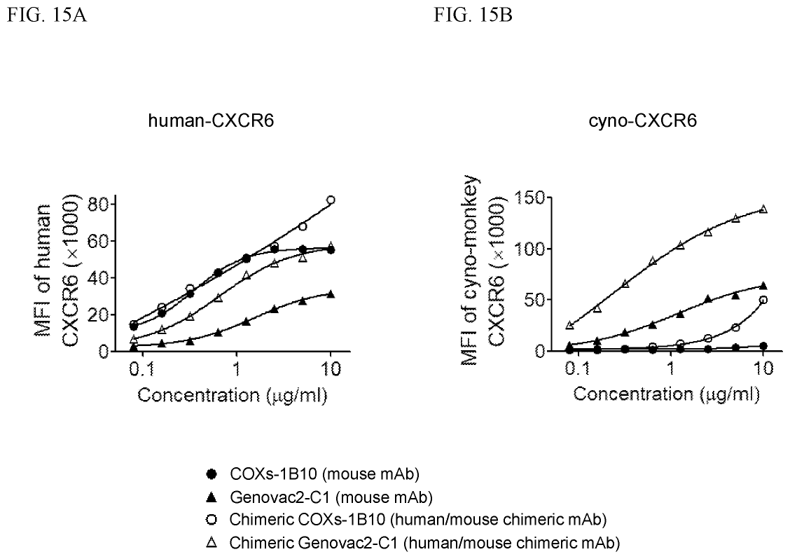

The experiments illustrated in FIGS. 15A-15B demonstrate that chimeric antibodies retain the binding capability and high affinity of full-length murine IgG1. COXs-1B10, the clone with the best affinity to human CXCR6 alone, and Genovac2-C1, the clone with the best affinities to human and cyno CXCR6, were expressed as human chimeric mAbs by grafting mouse-derived V_{H} and V_{L} into a human IgG1 antibody. Both mouse and human chimeric antibodies were tested for their binding to human and cyno CXCR6. As shown in FIGS. 15A and 15B, both clones maintained binding specificities and affinities. Chimeric Genovac2-C1 clone even displays improved properties compared to its mouse counterpart. Thus, the chimeric antibodies retain the binding capabilities and high affinities of the full-length murine IgG1.

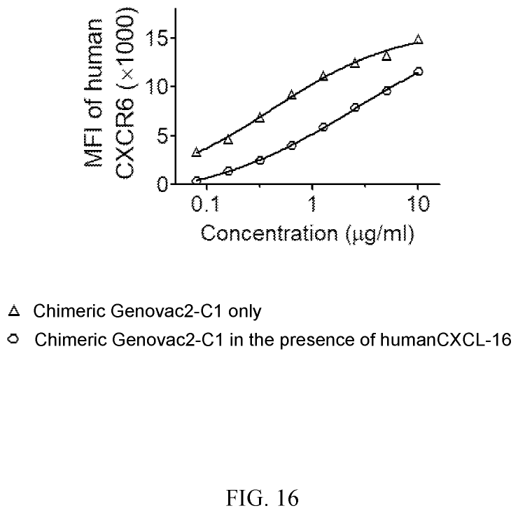

The experiments illustrated in FIG. 16 demonstrate that the human/mouse chimeric mAb retains the binding specificity and affinity in the presence of CXCL-16. Human CXCR6 over-expressing cells were pre-incubated with or without 1,500 ng/ml huCXCL-16 (Asn49-Pro137 of full length human CXCL-16, MW: 10 KD) for 5 min, then chimeric Genovac2-C1 was added and stained for additional 15 min at room temperature. After washing and staining with secondary antibody, cells were analyzed by flow cytometry. The ED₅₀ for huCXCL-16 binding to human CXCR6 is 10-50 ng/ml. Thus, 1,500 ng/ml huCXCL-16 is an overwhelming concentration for competing with chimeric Genovac2-C1. In the absence of huCXCL-16, the ED₅₀ for chimeric Genovac2-C1 binding to human CXCR6 was 0.5 μg/ml (FIG. 16). In the presence of huCXCL-16 (1,500 ng/ml), the ED₅₀ for chimeric Genovac2-C1 binding to human CXCR6 was 1.5 μg/ml (FIG. 16). Accordingly, the human/mouse chimeric mAb retains the binding specificity and affinity in the presence of CXCL-16.

### Example 7. Antibody-Dependent Cellular Cytotoxicity (ADCC) of Chimeric CXCR6 mAb

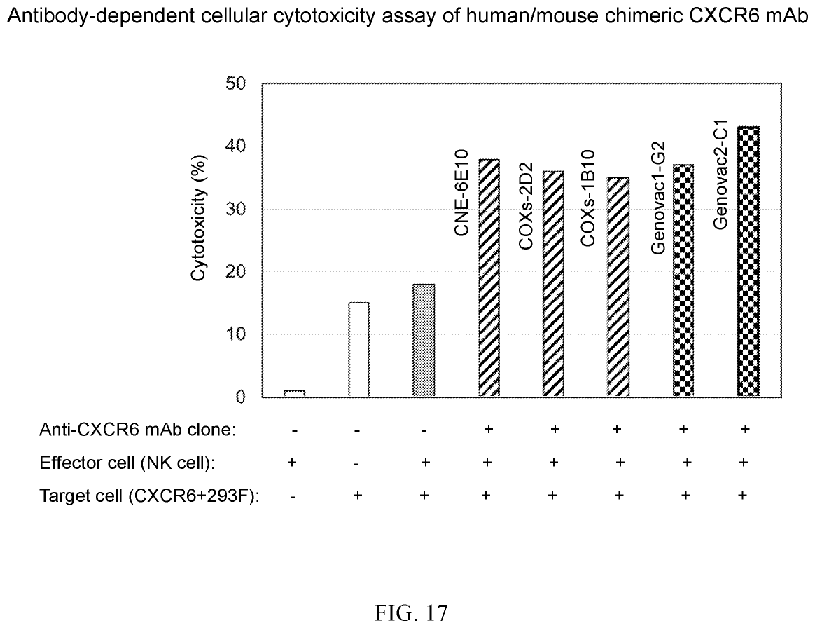

The experiments illustrated in FIG. 17 demonstrate that all tested antibodies can induce robust ADCC function in vitro, regardless the binding epitope (i.e., N-terminus of CXCR6, or extracellular loop (ECL). (N-terminus binders typically only recognize human CXCR6, while ECL-binders typically recognize both human and cyno-monkey CXCR6).

Clones CNE-6E10, COXs-2D2 and COXs-1B10 bind to human CXCR6, and clones Genovac1-G2 and Genovac2-C1 bind to both human and cyno monkey CXCR6. All clones were human/mouse chimeric antibodies with human IgG1 Fc.

The effector cells were isolated from peripheral blood mononuclear cells (PBMCs) prepared from fresh blood of a healthy human donor. Diluted with an equal volume of PBS, the blood was placed on top of Histopaque®-1077 density gradient cell separation medium (Sigma-Aldrich, St. Louis, MO) before being centrifuged at 400 g for 30 min (without break) at room temperature. After centrifugation, the monolayer was collected, washed, and resuspended in complete medium (RPMI-1640 containing 10% FBS). NK cells were isolated from the freshly prepared PBMCs using magnetic beads.

The target cells, CXCR6-overexpressing 293F (CXCR6⁺293F) cells, were seeded in a 96-well U-bottom plate for 15 min. Each well has either no mAb or 10 μg/ml of one of the different anti-CXCR6 mAb clones.

To measure spontaneous cell death, equal volumes of the NK cells were added into the wells of the 96-well plate. Each test well comprises co-cultured NK (effector) and CXCR6⁺293F (target) cells in the presence of an anti-CXCR6 mAb clone. A negative control well includes only effector cells, only target cells, or an effector and target cell co-culture in the absence of an anti-CXCR6 mAb clone. Each culture condition was duplicated. The 96-well plate was incubated in a cell-culture incubator at 37 C and 5% CO₂ for an additional 4 hours.

A standard curve was generated for quantifying lactate dehydrogenase (LDH) activities. Serially diluted target cells of known numbers were cultured in a complete medium at 37° C. and 5% CO₂ for 4 hours, in the presence of Triton X-100. The culture volume was the same as the ADCC reaction assay volume.

To measure LDH activities, the 96-well plate was centrifuged at the end of the cell culture process, and the supernatants (about 50 μl) were removed. The LDH activities were measured using an LDH assay kit, and the OD values were reported with a plate reader. Because LDH only leaks out from dead cells, LDH activities detected in the supernatants reflect cell death. The final death ratio of the cells from each testing well was normalized against the standard curve. The data (FIG. 17) demonstrates that all tested antibodies can induce robust ADCC function in vitro, regardless of the binding epitope.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

TABLE 1
CXCR6 Amino Acid Sequences

| | |
|---|---|
| SEQ ID | Amino Acid Sequence |

Human CXCR6

| | | |
|---|---|---|
| NO: 1 | Full length | MAEHDYHEDYGFSSFNDSSQEEHQDFLQFSKVFLPCMYLVVFVCGLVGNSLVLVISIFYHKLQSLTDV |
| | | FLVNLPLADLVFVCTLPFWAYAGIHEWVFGQVMCKSLLGIYTINFYTSMLILTCITVDRFIVVVKATKA |
| | | YNQQAKRMTWGKVTSLLIWVISLLVSLPQIIYGNVFNLDKLICGYHDEAISTVVLATQMTLGFFLPLLT |
| | | MIVCYSVIIKTLLHAGGFQKHRSLKIIFLVMAVFLLTQMPFNLMKFIRSTHWEYYAMTSFHYTIMVTEA |
| | | IAYLRACLNPVLYAFVSLKFRKNFWKLVKDIGCLPYLGVSHQWKSSEDNSKTFSASHNVEATSMFQL |
| NO: 2 | N-terminus | MAEHDYHEDYGFSSFNDSSQEEHQDFLQFSKV |
| NO: 3 | Extracellular | AGIHEWVFGQVMCKS |
| | Loop 1 | |
| NO: 4 | Extracellular | PQIIYGNVFNLDKLICGYHDEAI |
| | Loop 2 | |
| NO: 5 | Extracellular | YYAMTSFHYTIMVTEA |
| | Loop 3 | |

Cynomolgus monkey CXCR6

| | | |
|---|---|---|
| NO: 6 | Full length | MAEYDHYEDDGFLNSFNDSSQEEHQDFLQFRKVFLPCMYLVVFVCGLVGNSLVLVISIFYHKLQSLTD |
| | | VFLVNLPLADLVFVCTLPFWTYAGIHEWIFGQVMCKTLLGVYTINFYTSMLILTCITVDRFIVVVKATK |
| | | AYNQQAKRMTWGKVICLLIWVISLLVSLPQIIYGNVFNLDKLICGYHDEEISTVVLATQMTLGFFLPLL |
| | | AMIVCYSVIIKTLLHAGGFQKHRSLKIIFLVMAVFLLTQTPFNLVKLIRSTRWEYYAMTSFHYTIIVTE |
| | | AIAYLRACLNPVLYAFVSLKFRKNFWKLVKDIGCLPYLGVSHQWKSSEDNSKTFSASHNVEATSMFQL |
| NO: 7 | N-terminus | MAEYDHYEDDGFLNSFNDSSQEEHQDFLQFRKV |
| NO: 8 | Extracellular | AGIHEWIFGQVMCK |
| | Loop 1 | |
| NO: 9 | Extracellular | PQIIYGNVFNLDKLICGYHDEEI |
| | Loop 2 | |
| NO: 10 | Extracellular | YYAMTSFHYTIIVTEA |
| | Loop 3 | |

TABLE 2
V_{H} Amino Acid Sequences

| Name | SEQ ID | V_{H} Amino Acid Sequence |
|---|---|---|
| Genovac2-C1 | NO: 11 | DVKLVESGGGLVKPGGSLKLSCAASGFTFSSYTMSWVRQTPEKRLEWVATISSGGSYTYYPDSVKGRFTISRDNAKNT |
| | | LYLQMSSLKSEDTAMYYCTRGGGNYPFWFFDVWGAGTTVTVSS |
| Genovac1-G2 | NO: 12 | DVQLQESGPGLVKPSQSLSLTCSVTGYSITSGYYWNWIRQFPGNKLEWMGYIRYDGSNNYNPSLKNRISITRDTSKNQ |
| | | FFLKLNSVTTEDTATYYCARDGYSTYFDYWGQGTTLTVSS |
| Beacon2-G5 | NO: 13 | EVQLVESGGDLVKPGGSLKLSCAASGFTFSSYGMSWVRQTPDKRLEWVATISSRGSYTYYPDSVKGRLTISRENAKNT |
| | | LSLQMSSLKSEDTAMYYCARRVFPYWFFDVWGAGTTVTVSS |
| Beacon2-F1 | NO: 14 | EVQLVESGGDLVKPGGSLKLSCAASGFTFSSYGMSWVRQTPDKRLEWVATISSGGSYTYYPDSVKGRFPISRDNAKNT |
| | | LYQQMSSLNSEDTAMYYCARLYGNYPYFAMDYWGQGTSVTVSS |
| CNE-6E5-C1 | NO: 15 | QVTLKESGPGILQSSQILSLTCSFSGFSLITSGMGVSWIRKTSGKGLEWLAHIYWDDEKRYNPSLKSRLTISKGTSRN |
| | | QVFLRITSVDTTDTATYFCVRREDFGDGFDYWGQGTTLTVSP |
| CNGs-3B2 | NO: 16 | DVQLVESGGGLVQPGGSRTLSCAASGFTFSSFGMHWVRQAPEKGLEWVAYISSGSTTIYYADTVKGRFTISRDNPKNT |
| | | LFLQVTSLRSEDTAVYYCTRSHWPYWYFDVWGAGTTVTVSS |
| Beacon1-B2 | NO: 17 | EVQLQQSGXELVKPGASVKISCKASGYTFTDYYMNWVKLYHGKSLQWIGDINPNNGGTTYNQKFKGKATLTVDKSS |
| | | STAYMELRSLTSEDSAVYYCAKSRRNWGYAMDYWGQGTSVTVSS |
| CNGs-1B7, | NO: 18 | EVKLEESGGGLVRPGGSMKLSCVASGFTFSDYWMNWVRQSPEKGLEWLAQIRLNSGNYAARYAESVKGRFTISRDDS |
| Genovac2-H2 | | KNSVYLQMINLRAEDTGIYFCTSNAWLANWGQGTLVTVSA |
| CNE-6E10 | NO: 19 | EVQLVESGGGLVQPKGSLKLSCAASGFSFNTYAMNWVRQAPGKGLEWVARIRSKTNSYETYYADSVKDRFTISRDDS |
| | | ESMLYLQMNNLKTEDTAVYYCLSHGAFAYWGQGTLVTVSA |
| COXs-1B10 | NO: 20 | QVTLKESGPGILKASQTLSLTCSFSGFSLSTSGMGIGWIRQPSGKGPEWLAHIWWDDEKYYRPSLKSRLTVSKDTSRN |
| | | QVFLNITSVDTTDSASYYCVQRTPGRFAYWGQGTLVTVST |
| COXs-2D2 | NO: 21 | EVQLVESGGGLVQPKGSLKLSCTASGFSFDTYAMNWVRQAPGKGLEWIARIRSKSKNYATYYADSVKDRFTISRDDS |
| | | ANILYLQMNNLKTEDTAIYYCLRQGSFDYWGQGTTLTVSS |
| Beacon1-D10 | NO: 22 | EVQLMESGGGLVKPGGSRKLSCAASGFTFSDHGMHWVRQAPEKGLEWIAYINSGSSTIHYADSVKGRFTISRDNAKNT |
| | | LFLQMTSLRSEDTAMYYCVRPGFAYWGQGTLITVSA |
| Beacon1-G12, | NO: 23 | EIQLVQSGPELKKPGKTVKISCKASGYTFTTYGMSWVKQAPGKALKWMGWINTYSGVPTYADDFKGRFAFSLETSAS |
| COXs-4H5 | | TAYLQINNLKNEDTATYFCAIRRLSITLHGMDYWGQGTSVTVS |
| COXs-1B11 | NO: 24 | EVQLLEFGGGLVQPKGSLKLSCAASGFTFNTYAMNWVRQVQGKGLEWIARVRSKVKNYATYYADSVKDRFTISRDD |
| | | SESILYLQMNNLKTEDTGIYYCVRQGNFDVWGTGTSVTVSS |
| COXs-2D4 | NO: 25 | EVHLVESGGGLVQPKGSLKLSCTASGFSFDTYAMNWVRQAPGKGLEWIARIRSKSKNSATYYADSVKDRFTISRDDSA |
| | | NILYLQMNNLKTEDTAIYYCLRQGSFDYWGQGTTLTVSS |
| COXs-7A7 | NO: 26 | EVQLMESGGGLVKPGGSRKLSCAASGFTFSDYGMHWVRQAPEKGLEWIAYINSGSSTIHYADSVKGRFTISRDNAKNT |
| | | LFLQMTSLRSEDTAMYYCVRPGFAYWGQGTLITVSA |
| COXs-7E6 | NO: 27 | EVQLMESGGGLVKPGGSRKLSCAASGFTFSDHGMHWVRQAPEKGLEWIAYINSGSSTIHYADSVKGRFTISRDNAKNT |
| | | LFLHMTSLRSEDTAMYYCVRPGFAYWGQGTLITVSA |
| COXs-9D6 | NO: 28 | EVHLVESGGGLVQPKGSLKLSCTASGFSFDTYAMNWVRQAPGKGLEWVARIRSKSKNYATYYADSVTDRFTISRDDS |
| | | ANILYLQMNNLKTEDTAIYYCLSQGSFDYWGQGTTLTVSS |
| COXs-11E5 | NO: 29 | EVQLVESGGGLVQPKGSLKLSCTASGFSFDTYAMNWVRQAPGKGLEWVARIRSKSKNYATYYADSVKDRFTVSRDD |
| | | SANILYLQMNNLKTEDTAIYYCLRQGSFDYWGQGTTLTVSS |
| COXs-11H4 | NO: 30 | EVQLVESGGGLVQPKGSLKLSCAASGFTFNTYAMNWVRQVPGKGLEWIARMRSESKNFATYYADSVKDRFTISRDDS |
| | | ESILYLQMNNLKTEDTGMYYCVRQGNFDVWGTGTTVTVSS |
| COXs-12E7 | NO: 31 | EVHLVESGGGLVQPKGSLKLSCKASGFSFDTYAMNWVRQAPGKGLEWVARIRSKSKSYATYYADSVKDRFTISRDDS |
| | | ANILYLQMNNLKTEDTAIYYCLRQGSFDYWGQGTTLTVSS |
| CNE-7B1 | NO: 32 | QVQLQQSGPELVKPGTSVRISCKTSGYTFTSSYVHWVKRRPGQGLEWIGWIYPGNDYTKYNEKFKGKATLTADKSSST |
| | | AYMRLSSLASEDSAVYFCTRSYYGFDYWGQGTTLTVSS |
| CNF-2F8 | NO: 33 | EVQLVESGGGLVQPKGSLKLSCAASGFSFQTYAMNWVRQAPGKGLEWVARIRSKRNNYATYYADSVKDRFSISRDDS |
| | | ENMLYLQMNNLKTEDTAMYYCLSHGAFAYWGQGTLVTVSA |
| CNGs-1C1 | NO: 34 | EVQLVESGGGLVRPKGSLKLSCAASGFSFNAYAMNWVRQAPGKGLEWVARIRSKSNNYATYYADSVKDRFTISRDDS |
| | | ESMLYLQMNNLKTEDTAMYYCLSHGAFAYWGQGTLVTVSA |
| CNGs-2F11 | NO: 35 | EVQLQQSGPVLVKPGASVRMSCKASGYTFTEYYINWVKQGHGKSLEWIGIFNPYNGNTKYNQNFKGKATLTVDMSSS |
| | | TAYMELSSLTSEDSAVYYCSRPYSNFGQGTLVTVSA |
| CNE-6H7-G7 | NO: 36 | EVQLVESGGGLVQPKGSLKLSCAASGFSFNTYAMNWVRQAPGKGLEWVARIRSKSNNYGTYYADSVKDRFTISRDDS |
| | | ESMLYLKMNNLKTEDTAMYYCLSHGSSAYWGQGTLVTVSA |
| CNGs-1B7-E4 | NO: 37 | EVQLVESGGGLVQPRGSLKLSCAASGFTFNTYAMNWVRQAPGNGLEWVARIRSKSYNYATYHAGSVKDRFTISRDDS |
| | | QNMLYLQMNNLKTEDTAMYYCMRGQTYWGQGTLVTVSA |
| CNGs-1E1-G5 | NO: 38 | QVQLQQSGPELVKPGASVRISCKASGYTFTTSYIHWVKQRPGQGLEWIGWISPGNVNTRSNEKFKGKATLTADKSSNT |
| | | AYMQLSSLTSEDSAVYFCAKSYFGFDFWGQGTTLTVSS |
| CNGs-3G6-G5 | NO: 39 | EVQLQQSGPVLVKPGSSVKLSCKASGYTFTEYYINWVKQSHGKRLEWIGIFNPYNGNTKYNQKFKGKATLTVDKSSST |
| | | AYMELSSLTSEDSAVYYCSRPYSNFGQGTLVTVAA |
| CNGs-3H2-B7 | NO: 40 | EVQLVESGGGLVQPKGSLKLSCAASGVSFKTYAMTWVRQAPGKGLEWVARIRSKRYNYATYYADSVKDRFTISRDD |
| | | SESMLYLQMNNLKTEDTAMYYCMRQGAYWGQGTLVTVSA |
| Beacon2-A3 | NO: 41 | EVQIVESGGGLVQPKGSLKLSCAASGFSFNTYAMNWVRQAPGKGLEWVARIRTKRNNYVKFYADSVKDRFTISRDDS |
| | | ESMLYLQMNILKTEDTAMYYCVRQGAYWGQGTLVTVSA |
| Beacon2-A7 | NO: 42 | EMKLEESGGGLVQPGGSMKLSCVASGFTFSNYWMSWVRQSPEKGLQWVGQIRLKSDNYATHYAESVKGKFTISRDD |
| | | SKSSVYLQMNNLRAEDTGIYYCAPYSNWGYWGQGTTLTVSS |
| Beacon2-D5 | NO: 43 | EVQLVESGGGLVQPKGSLKLSCAASGFTFNTYAMNWVRQAPGKGLEWVARIRSKSNNYATYYADSVKDRFTISRDDS |
| | | QSMLYLQMNNLKTEDTAMYYCLSHGAFAYWGQGTLVTVSA |
| Beacon2-E5 | NO: 44 | EVQLVESGGGLVQPKGSLKLSCAASGFSFNTYAMNWVRQAPGKGLEWVARIRTKRNNYVKFYADSVKGRFTISRDD |
| | | SQSMLYLQMNNLKAEDTAIYYCVRQGAFWGQGTLVTVSA |
| Genovac1-D12 | NO: 45 | EVKLEESGGGLVQPGGSMKLSCVASGFTFSNYWMNWVRQSPEKGLKWVAQIRLKSDNYGTHYAESVKGRFTISRDD |
| | | SKGSVYLQMNNLRAEDTGIYYCTSNAWFAYWGQGTLVTVSA |
| Beacon1-A4 | NO: 46 | EVQLQQSGPVLVKPGASVKMSCKTSGYTFTDYYMNWVKQSHGKSLEWIGVIKPYSGRTSYDQKFKDKATLTVDKSS |
| | | STAYMELNSLTSEDSAVYYCAREGVSITTVGSSLYYYPMDYWGQGTSVTVSS |

TABLE 3
V_{L} Amino Acid Sequences

| Name | SEQ ID | V_{L} Amino Acid Sequence |
|---|---|---|
| Genovac2-C1 | NO: 47 | DIQMTQSPASLSASVGETVTITCRASENIYSYLAWYQQKQGKSPQLLVYNAKTLAEGVPSRFSGSGSGTQFSLKIKSLQP |
| | | EDFGSYYCQHHYGIPRTFGGGTKVETK |
| Genovac1-G2 | NO: 48 | DIQMTQSPASLSASVGETVTITCRASENIYSYLAWYQQKQGKSPQLLVYNAKTLAEGVPSRFSGSGSGTQFSLKINSLQP |
| | | EDFGSYYCQHHYGTPWTFGGGTKLEIK |
| Beacon2-G5 | NO: 49 | DIVMTQSHKFMSTSVGDRVSITCKASQDVSTAVAWYQQKSGQSPKLLIYSASYRYTGVPDRFTGSGSGTDFTFTISSVQ |
| | | AEDLAVYYCQQHYTTPYTFGGGTKLEIK |
| Beacon2-F1 | NO: 50 | DIVMTQSQKFMSTSVGDRVSITCKASQNVGTAVAWYQQKPGQSPKLLIFSASNRYTGVPDRFTGSGSGTDFTLTISNM |
| | | QSEDLADYFCQQYSSYPYTFGGGTKLEIK |
| CNE-6E5-C1 | NO: 51 | DVLMTQAPLSLPVSLGDQASISCRSSQTIVHSNGVTYLEWYLQKPGQSPKLLIYKVSNRFSGVPDRFSGSGSGTYFTLKI |
| | | SRVEAEDLGVYYCFQGSHVPPTFGGGTKLEIK |
| CNGs-3B2 | NO: 52 | DVQITQSPSYLAASPGETITINCRASKSISKYLAWYQEKPGKTNKLLIYSGSTLQSGNPSRFSGSGSGTDFTLTISRLEP |
| | | EDCAMYYCQQHNEFPWTFGGGTKLEIK |
| Beacon1-B2 | NO: 53 | DXVLTQSPASLAVSLGQRATISCRASESVSVRGTGLMHWYQQKPGYPPKLLIYAASNLESGVPARFSGRRSGTDFTLNI |
| | | HPVEEDDAATYYCQQSREYRTFGGGTKLEIK |
| CNGs-1B7 | NO: 54 | DIVMTQTAPSVPVTPGESVSISCRSSKSLLHSNGNTYLYWFLQRPGQSPQRLIYSVSNLASGVPDRFSGRGSGTDFTLTI |
| | | SRVEAEDVGVYYCMQSLEYPYTFGGGTKLEIK |
| CNE-6E10 | NO: 55 | DVVMTQTPLTLSVTIGQPASISCKSSQSLLDINGKTYLNWLLQRPGQSPKRLIYLVSKLDSGVPDRFTGSGSGTDFTLKI |
| | | SRVEAEDLGVYYCWQGTHFPQTFGGGTKLEIK |
| COXs-1B10 | NO: 56 | DIVMTQSPSSLAVSIGQKVIMSCKSSQSLLSSRTQKNYLAWYQQKPGQSPKLLVYFASIRESGVPDRFTGSGSGTDFTLT |
| | | ISTVQAEDLAVYYCQQYFLTPYTFGGGTKLEIK |
| COXs-2D2 | NO: 57 | DVVLTQTPLSLPVSLGDQASISCRSGQRIVHTDGNTYLYWYLQKPGQSPKLLIYRVSNRFSGVPDRFSGSGSGTDFTLKI |
| | | SRVEAEDMGIYYCFQGTHFPYTFGGGTKLEIK |
| Beacon1-D10 | NO: 58 | RRDDPRLHSPCLTVLEIQPPSSSDLIRTLCTMMETRIYIGPCRNQCQSPQLLIYRVSNRYSGVPDRFSGSGSGTDFTLKI |
| | | TRVEAEDMGVYYCFQATHDPFTFGGGTKLEIK |
| Beacon1-G12 | NO: 59 | DIQMTQSPASLSVSVGETVTITCRASENIYSNLAWYQQKQGKSPQLLVYGATNLADGVPSRFSGSGSGTQYSLKINSLQ |
| | | SEDFGSYYCQHFWGTPWTFGGGTKLEIK |
| COXs-1B11 | NO: 60 | DVVLTQTPLSLSVSLGDQASISCRSGQNIVHTDGNTYLYWSLQKPGQSPGLLIYKISNRFSGVPDRFSGSGSGTDFTLKI |
| | | SRVEAEDVGVYYCFQLTHFPYTFGGGTKLEIK |
| COXs-2D4 | NO: 61 | DVVMTQTPLSLPVSLGDQASISCRSSQRIVHTDGNTYLYWYLQKPGQSPKLLIYRVSNRFSGVPDRFSGSGSGTDFTLKI |
| | | TRVEAEDMGIYYCFQGTHFPYTFGGGTKLEIK |
| COXs-4H5 | NO: 62 | DVVMTQSPLSLPVSLGDQASIYCRSSQRIVHSDGNTYLYWYLQRPGQSPKLLIYRVSNRFSGVPDRFSGSGSGTDFTLKI |
| | | SRVEAEDMGIYYCFQGTHFPYTFGGGTKLEIK |
| COXs-7A7 | NO: 63 | DIVMTQTPLTLSTTVGQSAFISCRSSQSLLHRNGNTYLNWFLQRPGQSPQLLVYEIFERKSGVPDRFSGSGSGTDFTLKI |
| | | SRVEAEDLGVYYCMQATYEPLTFGAGTKLELK |
| COXs-7E6 | NO: 64 | DIVMTQTPLTLSITVGQSAFISCRSSQSLLHRNGNTYLNWFLQRPGQSPQLLVYEIFERKSGVPDRFSGSGSGTDFTLKI |
| | | SRVEAEDLGVYYCMQATYEPLTFGAGTKLELK |
| COXs-9D6, | NO: 65 | DVVMTQTPLSLPVSLGDQASISCRSSQRIVHTDGNTYLYWYLQKPGQSPKLLIYRVSNRFSGVPDRFSGSGSGTDFILKI |
| COXs-11E5 | | SRVEAEDMGIYYCFQGTHFPYTFGGGTKLEIK |
| COXs-11H4 | NO: 66 | DVVMTQAPLSLSVSLGDQASISCRSGQNIVHTDGNTYLYWSLQKPGQSPNLLIYKVSNRFSGVPDRFSGSGSGTDFTLK |
| | | INRVEAEDMGVYYCFQLTHVPYTFGGGTKLEIK |
| COXs-12E7 | NO: 67 | DVVMTQTPLSLPVSLGDQASIYCRSSQRIVHSDGNTYLYWYLQRPGQSPKLLIYRVSNRFSGVPDRFSGSGSGTDFTLKI |
| | | SRVEAEDMGIYYCFQGTHFPYTFGGGTKLEIK |
| CNE-7B1 | NO: 68 | DVLMTQTPLSLPVSLGDQASISCRSGQSIVRSRGNTYLEWYLQKPGQSPKLLIYKVSNRFSGVPDRFSGSGSGTDFTLKI |
| | | SRVEAEDLGVYYCFQGSHVPYTFGGGTKLEIK |
| CNF-2F8, | NO: 69 | DVVMTQTPLTLSVTIGQPASISCKSSQSLLDSDGKTYLNWLLQRPGQSPKRLIYLVSKLDSGVPDRFTGSGSGTDFTLKI |
| Beacon2-D5 | | SRVEAEDLGVYYCWQGTHFPQTFGGGTKLEIK |
| CNGs-1C1 | NO: 70 | DVVMTQTPLTLSVTIGQPASISCKSSQSLLYSDGKTYLNWLLQRPGQSPKRLIYLVSKLDSGVPDRFTGSGSGTDFTLKI |
| | | SRVEAEDLGVYYCWQATHFPQTFGGGTKLEIK |
| CNGs-2F11 | NO: 71 | DVLLTQIPLSLPVSLGDQASISCRSSQSIVHSNGNTYLEWYLQKPGQSPKLLIYKVSNRFSGVPDRFSGSGSGTDFTLKI |
| | | SRVGAEDLGVYYCFQGSHVPFTFGSGTKLEIK |
| CNE-6H7-G7 | NO: 72 | DVVLIQTPLTLSVTIGQPASISCRSSQSLLYSDGNTYLNWLLQRPGQSPKRLIYLVSELDSGVPDRFTGSGSGTDFTLKI |
| | | SRVEAEDLGVYYCWQGTHFPQTFGGGTKLEIK |
| CNGs-1B7-E4 | NO: 73 | DVVLTQTPLSLPVNIGDQASISCKSTKSLLNSDGFTYLDWHLQKPGQSPQLLIYLVSNRFSGVPDRFSGSGSGTDFTLKI |
| | | SRVEAEDLGVYYCFQSNYLPFTFGSGTKLEMK |
| CNGs-1E1-G5 | NO: 74 | DVLMTQTPLSLPVSLGDQASISCRSSQSIVHSNGNTYLEWYVQKPGQSPKLLIYKVSNRSSGVPDRFSGSGSGTDFTLKI |
| | | SRVEAEDLGVYYCFQGSHVPYTFGGGTKLEIK |
| CNGs-3G6-G5 | NO: 75 | DVLMTQTPLSLPVSLGDQASISCRSSQSLVHSNGNTFLEWFLQKPGQSPKLLIYKVSNRFSGVPDRFSGSGSGTDFTLKI |
| | | SRVEAEDLGVYYCFQGSHVPFTFGSGTKLEIK |
| CNGs-3H2-B7 | NO: 76 | DVVLTQTPLSLPVNIGDQASISCKSTKSLLNSDGFTYLDWYLQKPGQSPHLLIYLVSNRFSGVPDRFSGSGSGTDFTLKI |
| | | SRVEAEDLGVYYCFQSNYLPFTFGSGTKLEIK |
| Beacon2-A3 | NO: 77 | DVVLTQTPLSLPVNIGDQASISCKSTKSLLNSDGFTYLDWYLQKPGQSPQLLIYLVSNRFSGVPDRFSGSGSGTDFTLKI |
| | | SRVEAEDLGVYFCFQSNYLPYTFGGGTKLEIK |
| Beacon2-A7 | NO: 78 | DIVMTQAAPSIPVTPGGSVSISCRSTKSLLHTNGNTYLYWFLQRPGQSPQVLIYRMSNLASGVPDRFSGSGSGTAFTLRI |
| | | SRVEAEDVGVYYCMQYLEYPYTFGSGTKLEIK |
| Beacon2-E5 | NO: 79 | DVVLTQTPLSLPVNIGDQASISCKSTKSLLNSDGFTYLDWYLQKPGQSPQLLIYLVSNRFSGVPDRFSGSGSGTDFTLRI |
| | | SRVEAEDLGVYYCFQSNYLPYTFGGGTKLEIK |
| Genovac1- | NO: 80 | DIVMTQAAPSVPVTPGESVSISCRSSKSLLHSNGNTYLFWFLQRPGQSPQRLISYMSNLASGVPDRFSGRGSGTDFTLRI |
| D12 | | SRVEAEDVGVYYCLLSLEYPYTFGSGTRLEIT |
| Genovac2-H2 | NO: 81 | DIQMTQSPASLSVSVGETVTITCRASENIYNNLAWYQQKQGKSPQLLVYAATNLADGVPSRFSGSGSGTQYSLKINSLQ |
| | | SEDFGSYYCQHFWGTPWTFGGGTKLEIK |
| Beacon1-A4 | NO: 82 | DIVLTQSPATLSVTPGASVSLSCRASQSISNNLHWYQQKSHESLRLLIKYASQSVSGIPSRFSGSGSGTDFTLTINSVET |
| | | EDFGMYFCQQSNSWPRTFGGGTKLEIK |

TABLE 5
HCDR Amino Acid Sequences (IMGT)

| Name | SEQ ID | HCDR1 | SEQ ID | HCDR2 | SEQ ID | HCDR3 |
|---|---|---|---|---|---|---|
| Genovac2-C1 | NO: 83 | GFTFSSYT | NO: 84 | ISSGGSYT | NO: 85 | TRGGGNYPFWFFDV |
| Genovac1-G2 | NO: 86 | GYSITSGYY | NO: 87 | IRYDGSN | NO: 88 | ARDGYSTYFDY |
| Beacon2-G5 | NO: 89 | GFTFSSYG | NO: 90 | ISSRGSYT | NO: 91 | ARRVFPYWFFDV |
| Beacon2-F1 | NO: 89 | GFTFSSYG | NO: 84 | ISSGGSYT | NO: 92 | ARLYGNYPYFAMDY |
| CNE-6E5-C1 | NO: 93 | GFSLITSGMG | NO: 94 | IYWDDEK | NO: 95 | VRREDFGDGFDY |
| CNGs-3B2 | NO: 96 | GFTFSSFG | NO: 97 | ISSGSTTI | NO: 98 | TRSHWPYWYFDV |
| Beacon1-B2 | NO: 99 | GYTFTDYY | NO: 100 | INPNNGGT | NO: 101 | AKSRRNWGYAMDY |
| CNGs-1B7 | NO: 102 | GFTFSDYW | NO: 103 | IRLNSGNYAA | NO: 104 | TSNAWLAN |
| CNE-6E10 | NO: 105 | GFSFNTYA | NO: 106 | IRSKTNSYET | NO: 107 | LSHGAFAY |
| COXs-1B10 | NO: 108 | GFSLSTSGMG | NO: 109 | IWWDDEK | NO: 110 | VQRTPGRFAY |
| COXs-2D2 | NO: 111 | GFSFDTYA | NO: 112 | IRSKSKNYAT | NO: 113 | LRQGSFDY |
| Beacon1-D10 | NO: 114 | GFTFSDHG | NO: 115 | INSGSSTI | NO: 116 | VRPGFAY |
| Beacon1-G12 | NO: 117 | GYTFTTYG | NO: 118 | INTYSGVP | NO: 119 | AIRRLSITLHGMDY |
| COXs-1B11 | NO: 120 | GFTFNTYA | NO: 121 | VRSKVKNYAT | NO: 122 | VRQGNFDV |
| COXs-2D4 | NO: 111 | GFSFDTYA | NO: 123 | IRSKSKNSAT | NO: 113 | LRQGSFDY |
| COXs-4H5 | NO: 117 | GYTFTTYG | NO: 118 | INTYSGVP | NO: 119 | AIRRLSITLHGMDY |
| COXs-7A7 | NO: 124 | GFTFSDYG | NO: 115 | INSGSSTI | NO: 116 | VRPGFAY |
| COXs-7E6 | NO: 114 | GFTFSDHG | NO: 115 | INSGSSTI | NO: 116 | VRPGFAY |
| COXs-9D6 | NO: 111 | GFSFDTYA | NO: 112 | IRSKSKNYAT | NO: 125 | LSQGSFDY |
| COXs-11E5 | NO: 111 | GFSFDTYA | NO: 112 | IRSKSKNYAT | NO: 113 | LRQGSFDY |
| COXs-11H4 | NO: 120 | GFTFNTYA | NO: 126 | MRSESKNFAT | NO: 122 | VRQGNFDV |
| COXs-12E7 | NO: 111 | GFSFDTYA | NO: 127 | IRSKSKSYAT | NO: 113 | LRQGSFDY |
| CNE-7B1 | NO: 128 | GYTFTSSY | NO: 129 | IYPGNDYT | NO: 130 | TRSYYGFDY |
| CNF-2F8 | NO: 131 | GFSFQTYA | NO: 132 | IRSKRNNYAT | NO: 107 | LSHGAFAY |
| CNGs-1C1 | NO: 133 | GFSFNAYA | NO: 134 | IRSKSNNYAT | NO: 107 | LSHGAFAY |
| CNGs-2F11 | NO: 135 | GYTFTEYY | NO: 136 | FNPYNGNT | NO: 137 | SRPYSN |
| CNE-6H7-G7 | NO: 105 | GFSFNTYA | NO: 138 | IRSKSNNYGT | NO: 139 | LSHGSSAY |
| CNGs-1B7-E4 | NO: 120 | GFTFNTYA | NO: 140 | IRSKSYNYAT | NO: 141 | MRGQTY |
| CNGs-1E1-G5 | NO: 142 | GYTFTTSY | NO: 143 | ISPGNVNT | NO: 144 | AKSYFGFDF |
| CNGs-3G6-G5 | NO: 135 | GYTFTEYY | NO: 136 | FNPYNGNT | NO: 137 | SRPYSN |
| CNGs-3H2-B7 | NO: 145 | GVSFKTYA | NO: 146 | IRSKRYNYAT | NO: 147 | MRQGAY |
| Beacon2-A3 | NO: 105 | GFSFNTYA | NO: 148 | IRTKRNNYVK | NO: 149 | VRQGAY |
| Beacon2-A7 | NO: 150 | GFTFSNYW | NO: 151 | IRLKSDNYAT | NO: 152 | APYSNWGY |
| Beacon2-D5 | NO: 120 | GFTFNTYA | NO: 134 | IRSKSNNYAT | NO: 107 | LSHGAFAY |
| Beacon2-E5 | NO: 105 | GFSFNTYA | NO: 148 | IRTKRNNYVK | NO: 153 | VRQGAF |
| Genovac1-D12 | NO: 150 | GFTFSNYW | NO: 154 | IRLKSDNYGT | NO: 155 | TSNAWFAY |
| Genovac2-H2 | NO: 102 | GFTFSDYW | NO: 103 | IRLNSGNYAA | NO: 104 | TSNAWLAN |
| Beacon1-A4 | NO: 99 | GYTFTDYY | NO: 156 | IKPYSGRT | NO: 157 | AREGVSITTVGSSLY |
| | | | | | | YYPMDY |

TABLE 6
LCDR Amino Acid Sequences (IMGT)

| Name | SEQ ID | LCDR1 | SEQ ID | LCDR2 | SEQ ID | LCDR3 |
|---|---|---|---|---|---|---|
| Genovac2-C1 | NO: 158 | ENIYSY | NO: 159 | NAK | NO: 160 | QHHYGIPRT |
| Genovac1-G2 | NO: 158 | ENIYSY | NO: 159 | NAK | NO: 161 | QHHYGTPWT |
| Beacon2-G5 | NO: 162 | QDVSTA | NO: 163 | SAS | NO: 164 | QQHYTTPYT |
| Beacon2-F1 | NO: 165 | QNVGTA | NO: 163 | SAS | NO: 166 | QQYSSYPYT |
| CNE-6E5-C1 | NO: 167 | QTIVHSNGVTY | NO: 168 | KVS | NO: 169 | FQGSHVPPT |
| CNGs-3B2 | NO: 170 | KSISKY | NO: 171 | SGS | NO: 172 | QQHNEFPWT |
| Beacon1-B2 | NO: 173 | ESVSVRGTGL | NO: 174 | AAS | NO: 175 | QQSREYRT |
| CNGs-1B7 | NO: 176 | KSLLHSNGNTY | NO: 177 | SVS | NO: 178 | MQSLEYPYT |
| CNE-6E10 | NO: 179 | QSLLDINGKTY | NO: 180 | LVS | NO: 181 | WQGTHFPQT |
| COXs-1B10 | NO: 182 | QSLLSSRTQKNY | NO: 183 | FAS | NO: 184 | QQYFLTPYT |
| COXs-2D2 | NO: 185 | QRIVHTDGNTY | NO: 186 | RVS | NO: 187 | FQGTHFPYT |
| Beacon1-D10 | NO: 188 | RTLCTMMETRI | NO: 186 | RVS | NO: 189 | FQATHDPFT |
| Beacon1-G12 | NO: 190 | ENIYSN | NO: 191 | GAT | NO: 192 | QHFWGTPWT |
| COXs-1B11 | NO: 193 | QNIVHTDGNTY | NO: 194 | KIS | NO: 195 | FQLTHFPYT |
| COXs-2D4 | NO: 185 | QRIVHTDGNTY | NO: 186 | RVS | NO: 187 | FQGTHFPYT |
| COXs-4H5 | NO: 196 | QRIVHSDGNTY | NO: 186 | RVS | NO: 187 | FQGTHFPYT |
| COXs-7A7 | NO: 197 | QSLLHRNGNTY | NO: 198 | EIF | NO: 199 | MQATYEPLT |
| COXs-7E6 | NO: 197 | QSLLHRNGNTY | NO: 198 | EIF | NO: 199 | MQATYEPLT |
| COXs-9D6 | NO: 185 | QRIVHTDGNTY | NO: 186 | RVS | NO: 187 | FQGTHFPYT |
| COXs-11E5 | NO: 185 | QRIVHTDGNTY | NO: 186 | RVS | NO: 187 | FQGTHFPYT |
| COXs-11H4 | NO: 193 | QNIVHTDGNTY | NO: 168 | KVS | NO: 200 | FQLTHVPYT |
| COXs-12E7 | NO: 196 | QRIVHSDGNTY | NO: 186 | RVS | NO: 187 | FQGTHFPYT |
| CNE-7B1 | NO: 201 | QSIVRSRGNTY | NO: 168 | KVS | NO: 202 | FQGSHVPYT |
| CNF-2F8 | NO: 203 | QSLLDSDGKTY | NO: 180 | LVS | NO: 181 | WQGTHFPQT |
| CNGs-1C1 | NO: 204 | QSLLYSDGKTY | NO: 180 | LVS | NO: 205 | WQATHFPQT |
| CNGs-2F11 | NO: 206 | QSIVHSNGNTY | NO: 168 | KVS | NO: 207 | FQGSHVPFT |
| CNE-6H7-G7 | NO: 208 | QSLLYSDGNTY | NO: 180 | LVS | NO: 181 | WQGTHFPQT |
| CNGs-1B7-E4 | NO: 209 | KSLLNSDGFTY | NO: 180 | LVS | NO: 210 | FQSNYLPFT |
| CNGs-1E1-G5 | NO: 206 | QSIVHSNGNTY | NO: 168 | KVS | NO: 202 | FQGSHVPYT |
| CNGs-3G6-G5 | NO: 211 | QSLVHSNGNTF | NO: 168 | KVS | NO: 207 | FQGSHVPFT |
| CNGs-3H2-B7 | NO: 209 | KSLLNSDGFTY | NO: 180 | LVS | NO: 210 | FQSNYLPFT |
| Beacon2-A3 | NO: 209 | KSLLNSDGFTY | NO: 180 | LVS | NO: 212 | FQSNYLPYT |
| Beacon2-A7 | NO: 213 | KSLLHTNGNTY | NO: 214 | RMS | NO: 215 | MQYLEYPYT |
| Beacon2-D5 | NO: 203 | QSLLDSDGKTY | NO: 180 | LVS | NO: 181 | WQGTHFPQT |
| Beacon2-E5 | NO: 209 | KSLLNSDGFTY | NO: 180 | LVS | NO: 212 | FQSNYLPYT |
| Genovac1-D12 | NO: 176 | KSLLHSNGNTY | NO: 216 | YMS | NO: 217 | LLSLEYPYT |
| Genovac2-H2 | NO: 218 | ENIYNN | NO: 219 | AAT | NO: 192 | QHFWGTPWT |
| Beacon1-A4 | NO: 220 | QSISNN | NO: 221 | YAS | NO: 222 | QQSNSWPRT |

TABLE 7
HCDR Amino Acid Sequences (Kabat)

| Name | SEQ ID | HCDR1 | SEQ ID | HCDR2 | SEQ ID | HCDR3 |
|---|---|---|---|---|---|---|
| Genovac2-C1 | NO: 223 | GFTFSSYTMS | NO: 224 | TISSGGSYTYYPDSVKG | NO: 225 | GGGNYPFWFFDV |
| Genovac1-G2 | NO: 86 | GYSITSGYY | NO: 226 | YIRYDGSNNYNPSLKN | NO: 227 | DGYSTYFDY |
| Beacon2-G5 | NO: 228 | GFTFSSYGMS | NO: 229 | TISSRGSYTYYPDSVKG | NO: 230 | RVFPYWFFDV |
| Beacon2-F1 | NO: 228 | GFTFSSYGMS | NO: 224 | TISSGGSYTYYPDSVKG | NO: 231 | LYGNYPYFAMDY |
| CNE-6E5-C1 | NO: 232 | GFSLITSGMGVS | NO: 233 | HIYWDDEKRYNPSLKS | NO: 234 | REDFGDGFDY |
| CNGs-3B2 | NO: 235 | GFTFSSFGMH | NO: 236 | YISSGSTTIYYADTVKG | NO: 237 | SHWPYWYFDV |
| Beacon1-B2 | NO: 238 | GYTFTDYYMN | NO: 239 | DINPNNGGTTYNQKFKG | NO: 240 | SRRNWGYAMDY |
| CNGs-1B7 | NO: 241 | GFTFSDYWMN | NO: 242 | QIRLNSGNYAARYAESVKG | NO: 243 | NAWLAN |
| CNE-6E10 | NO: 244 | GFSFNTYAMN | NO: 245 | RIRSKTNSYETYYADSVKD | NO: 246 | HGAFAY |
| COXs-1B10 | NO: 247 | GFSLSTSGMGIG | NO: 248 | HIWWDDEKYYRPSLKS | NO: 249 | RTPGRFAY |
| COXs-2D2 | NO: 250 | GFSFDTYAMN | NO: 251 | RIRSKSKNYATYYADSVKD | NO: 252 | QGSFDY |
| Beacon1-D10 | NO: 253 | GFTFSDHGMH | NO: 254 | YINSGSSTIHYADSVKG | NO: 255 | PGFAY |
| Beacon1-G12 | NO: 256 | GYTFTTYGMS | NO: 257 | WINTYSGVPTYADDFKG | NO: 258 | RRLSITLHGMDY |
| COXs-1B11 | NO: 259 | GFTFNTYAMN | NO: 260 | RVRSKVKNYATYYADSVKD | NO: 261 | QGNFDV |
| COXs-2D4 | NO: 250 | GFSFDTYAMN | NO: 262 | RIRSKSKNSATYYADSVKD | NO: 252 | QGSFDY |
| COXs-4H5 | NO: 256 | GYTFTTYGMS | NO: 257 | WINTYSGVPTYADDFKG | NO: 258 | RRLSITLHGMDY |
| COXs-7A7 | NO: 263 | GFTFSDYGMH | NO: 254 | YINSGSSTIHYADSVKG | NO: 255 | PGFAY |
| COXs-7E6 | NO: 253 | GFTFSDHGMH | NO: 254 | YINSGSSTIHYADSVKG | NO: 255 | PGFAY |
| COXs-9D6 | NO: 250 | GFSFDTYAMN | NO: 264 | RIRSKSKNYATYYADSVTD | NO: 252 | QGSFDY |
| COXs-11E5 | NO: 250 | GFSFDTYAMN | NO: 251 | RIRSKSKNYATYYADSVKD | NO: 252 | QGSFDY |
| COXs-11H4 | NO: 259 | GFTFNTYAMN | NO: 265 | RMRSESKNFATYYADSVKD | NO: 261 | QGNFDV |
| COXs-12E7 | NO: 250 | GFSFDTYAMN | NO: 266 | RIRSKSKSYATYYADSVKD | NO: 252 | QGSFDY |
| CNE-7B1 | NO: 267 | GYTFTSSYVH | NO: 268 | WIYPGNDYTKYNEKFKG | NO: 269 | SYYGFDY |
| CNF-2F8 | NO: 270 | GFSFQTYAMN | NO: 271 | RIRSKRNNYATYYADSVKD | NO: 246 | HGAFAY |
| CNGs-1C1 | NO: 272 | GFSFNAYAMN | NO: 273 | RIRSKSNNYATYYADSVKD | NO: 246 | HGAFAY |
| CNGs-2F11 | NO: 274 | GYTFTEYYIN | NO: 275 | IFNPYNGNTKYNQNFKG | NO: 276 | PYSN |
| CNE-6H7-G7 | NO: 244 | GFSFNTYAMN | NO: 277 | RIRSKSNNYGTYYADSVKD | NO: 278 | HGSSAY |
| CNGs-1B7-E4 | NO: 259 | GFTFNTYAMN | NO: 279 | RIRSKSYNYATYHAGSVKD | NO: 280 | GQTY |
| CNGs-1E1-G5 | NO: 281 | GYTFTTSYIH | NO: 282 | WISPGNVNTRSNEKFKG | NO: 283 | SYFGFDF |
| CNGs-3G6-G5 | NO: 274 | GYTFTEYYIN | NO: 284 | IFNPYNGNTKYNQKFKG | NO: 276 | PYSN |
| CNGs-3H2-B7 | NO: 285 | GVSFKTYAMT | NO: 286 | RIRSKRYNYATYYADSVKD | NO: 287 | QGAY |
| Beacon2-A3 | NO: 244 | GFSFNTYAMN | NO: 288 | RIRTKRNNYVKFYADSVKD | NO: 287 | QGAY |
| Beacon2-A7 | NO: 289 | GFTFSNYWMS | NO: 290 | QIRLKSDNYATHYAESVKG | NO: 291 | YSNWGY |
| Beacon2-D5 | NO: 259 | GFTFNTYAMN | NO: 273 | RIRSKSNNYATYYADSVKD | NO: 246 | HGAFAY |
| Beacon2-E5 | NO: 244 | GFSFNTYAMN | NO: 292 | RIRTKRNNYVKFYADSVKG | NO: 293 | QGAF |
| Genovac1-D12 | NO: 294 | GFTFSNYWMN | NO: 295 | QIRLKSDNYGTHYAESVKG | NO: 296 | NAWFAY |
| Genovac2-H2 | NO: 241 | GFTFSDYWMN | NO: 242 | QIRLNSGNYAARYAESVKG | NO: 243 | NAWLAN |
| Beacon1-A4 | NO: 238 | GYTFTDYYMN | NO: 297 | VIKPYSGRTSYDQKFKD | NO: 298 | EGVSITTVGSSLYY |
| | | | | | | YPMDY |

TABLE 8
LCDR Amino Acid Sequences (Kabat)

| Name | SEQ ID | LCDR1 | SEQ ID | LCDR2 | SEQ ID | LCDR3 |
|---|---|---|---|---|---|---|
| Genovac2-C1 | NO: 299 | RASENIYSYLA | NO: 300 | NAKTLAE | NO: 160 | QHHYGIPRT |
| Genovac1-G2 | NO: 299 | RASENIYSYLA | NO: 300 | NAKTLAE | NO: 161 | QHHYGTPWT |
| Beacon2-G5 | NO: 301 | KASQDVSTAVA | NO: 302 | SASYRYT | NO: 164 | QQHYTTPYT |
| Beacon2-F1 | NO: 303 | KASQNVGTAVA | NO: 304 | SASNRYT | NO: 166 | QQYSSYPYT |
| CNE-6E5-C1 | NO: 305 | RSSQTIVHSNGVTYLE | NO: 306 | KVSNRFS | NO: 169 | FQGSHVPPT |
| CNGs-3B2 | NO: 307 | RASKSISKYLA | NO: 308 | SGSTLQS | NO: 172 | QQHNEFPWT |
| Beacon1-B2 | NO: 309 | RASESVSVRGTGLMH | NO: 310 | AASNLES | NO: 175 | QQSREYRT |
| CNGs-1B7 | NO: 311 | RSSKSLLHSNGNTYLY | NO: 312 | SVSNLAS | NO: 178 | MQSLEYPYT |
| CNE-6E10 | NO: 313 | KSSQSLLDINGKTYLN | NO: 314 | LVSKLDS | NO: 181 | WQGTHFPQT |
| COXs-1B10 | NO: 315 | KSSQSLLSSRTQKNYLA | NO: 316 | FASIRES | NO: 184 | QQYFLTPYT |
| COXs-2D2 | NO: 317 | RSGQRIVHTDGNTYLY | NO: 318 | RVSNRFS | NO: 187 | FQGTHFPYT |
| Beacon1-D10 | NO: 188 | RTLCTMMETRI | NO: 318 | RVSNRFS | NO: 189 | FQATHDPFT |
| Beacon1-G12 | NO: 319 | RASENIYSNLA | NO: 320 | GATNLAD | NO: 192 | QHFWGTPWT |
| COXs-1B11 | NO: 321 | RSGQNIVHTDGNTYLY | NO: 322 | KISNRFS | NO: 195 | FQLTHFPYT |
| COXs-2D4 | NO: 323 | RSSQRIVHTDGNTYLY | NO: 318 | RVSNRFS | NO: 187 | FQGTHFPYT |
| COXs-4H5 | NO: 324 | RSSQRIVHSDGNTYLY | NO: 318 | RVSNRFS | NO: 187 | FQGTHFPYT |
| COXs-7A7 | NO: 325 | RSSQSLLHRNGNTYLN | NO: 326 | EIFERKS | NO: 199 | MQATYEPLT |
| COXs-7E6 | NO: 325 | RSSQSLLHRNGNTYLN | NO: 326 | EIFERKS | NO: 199 | MQATYEPLT |
| COXs-9D6 | NO: 323 | RSSQRIVHTDGNTYLY | NO: 318 | RVSNRFS | NO: 187 | FQGTHFPYT |
| COXs-11E5 | NO: 323 | RSSQRIVHTDGNTYLY | NO: 318 | RVSNRFS | NO: 187 | FQGTHFPYT |
| COXs-11H4 | NO: 321 | RSGQNIVHTDGNTYLY | NO: 306 | KVSNRFS | NO: 200 | FQLTHVPYT |
| COXs-12E7 | NO: 324 | RSSQRIVHSDGNTYLY | NO: 318 | RVSNRFS | NO: 187 | FQGTHFPYT |
| CNE-7B1 | NO: 327 | RSGQSIVRSRGNTYLE | NO: 306 | KVSNRFS | NO: 202 | FQGSHVPYT |
| CNF-2F8 | NO: 328 | KSSQSLLDSDGKTYLN | NO: 314 | LVSKLDS | NO: 181 | WQGTHFPQT |
| CNGs-1C1 | NO: 329 | KSSQSLLYSDGKTYLN | NO: 314 | LVSKLDS | NO: 205 | WQATHFPQT |
| CNGs-2F11 | NO: 330 | RSSQSIVHSNGNTYLE | NO: 306 | KVSNRFS | NO: 207 | FQGSHVPFT |
| CNE-6H7-G7 | NO: 331 | RSSQSLLYSDGNTYLN | NO: 332 | LVSELDS | NO: 181 | WQGTHFPQT |
| CNGs-1B7-E4 | NO: 333 | KSTKSLLNSDGFTYLD | NO: 334 | LVSNRFS | NO: 210 | FQSNYLPFT |
| CNGs-1E1-G5 | NO: 330 | RSSQSIVHSNGNTYLE | NO: 335 | KVSNRSS | NO: 202 | FQGSHVPYT |
| CNGs-3G6-G5 | NO: 336 | RSSQSLVHSNGNTFLE | NO: 306 | KVSNRFS | NO: 207 | FQGSHVPFT |
| CNGs-3H2-B7 | NO: 333 | KSTKSLLNSDGFTYLD | NO: 334 | LVSNRFS | NO: 210 | FQSNYLPFT |
| Beacon2-A3 | NO: 333 | KSTKSLLNSDGFTYLD | NO: 334 | LVSNRFS | NO: 212 | FQSNYLPYT |
| Beacon2-A7 | NO: 337 | RSTKSLLHTNGNTYLY | NO: 338 | RMSNLAS | NO: 215 | MQYLEYPYT |
| Beacon2-D5 | NO: 328 | KSSQSLLDSDGKTYLN | NO: 314 | LVSKLDS | NO: 181 | WQGTHFPQT |
| Beacon2-E5 | NO: 333 | KSTKSLLNSDGFTYLD | NO: 334 | LVSNRFS | NO: 212 | FQSNYLPYT |
| Genovac1-D12 | NO: 339 | RSSKSLLHSNGNTYLF | NO: 340 | YMSNLAS | NO: 217 | LLSLEYPYT |
| Genovac2-H2 | NO: 341 | RASENIYNNLA | NO: 342 | AATNLAD | NO: 192 | QHFWGTPWT |
| Beacon1-A4 | NO: 343 | RASQSISNNLH | NO: 344 | YASQSVS | NO: 222 | QQSNSWPRT |

TABLE 9
V_{H} and V_{L} Amino Acid Sequences of Checkpoint Inhibitors

| SEQ ID | Amino Acid Sequence |
|---|---|
| NO: 349 | QVQLVQSGVEVKKPGASVKVSCKASGYTFTNYYMYWVRQAPGQGLEWMGGINPSNGGTNFN |
| | EKFKNRVTLTTDSSTTTAYMELKSLQFDDTAVYYCARRDYRFDMGFDYWGQGTTVTVSS |
| NO: 350 | EIVLTQSPATLSLSPGERATLSCRASKGVSTSGYSYLHWYQQKPGQAPRLLIYLASYLESGVPAR |
| | FSGSGSGTDFTLTISSLEPEDFAVYYCQHSRDLPLTFGGGTKVEIK |
| NO: 351 | QVQLVESGGGVVQPGRSLRLDCKASGITFSNSGMHWVRQAPGKGLEWVAVIWYDGSKRYYAD |
| | SVKGRFTISRDNSKNTLFLQMNSLRAEDTAVYYCATNDDYWGQGTLVTVSS |
| NO: 352 | EIVLTQSPATLSLSPGERATLSCRASQSVSSYLAWYQQKPGQAPRLLIYDASNRATGIPARFSGSG |
| | SGTDFTLTISSLEPEDFAVYYCQQSSNWPRTFGQGTKVEIK |
| NO: 353 | QVQLVQSGAEVKKPGSSVKVSCKASGGTFSSYAISWVRQAPGQGLEWMGGIIPIFDTANYAQKF |
| | QGRVTITADESTSTAYMELSSLRSEDTAVYYCARPGLAAAYDTGSLDYWGQGTLVTVSS |
| NO: 354 | EIVLTQSPATLSLSPGERATLSCRASQSVRSYLAWYQQKPGQAPRLLIYDASNRATGIPARFSGSG |
| | SGTDFTLTISSLEPEDFAVYYCQQRNYWPLTFGQGTKVEIK |
| NO: 355 | EVQLVESGGGLVQPGGSLRLSCAASGFAFSRYDMSWVRQAPGKGLESVAYISGGGANTYYLDN |
| | VKGRFTISRDNAKNSLYLQMNSLRAEDTAVYYCASPYLSYFDVWGQGTLVTVSS |
| NO: 356 | EIVMTQSPATLSVSPGERATLSCRASQSLSDYLHWYQQKPGQAPRLLIKSASQSISGIPARFSGSG |
| | SGTEFTLTISSLQSEDFAVYYCQNGHSFPYTFGQGTKLEIK |
| NO: 357 | EVQLVESGGGLVQPGGSLRLSCAASGFTFSRYWMSWVRQAPGKGLEWVANIKQDGSEKYYVD |
| | SVKGRFTISRDNAKNSLYLQMNSLRAEDTAVYYCAREGGWFGELAFDYWGQGTLVTVSS |
| NO: 358 | EIVLTQSPGTLSLSPGERATLSCRASQRVSSSYLAWYQQKPGQAPRLLIYDASSRATGIPDRFSGS |
| | GSGTDFTLTISRLEPEDFAVYYCQQYGSLPWTFGQGTKVEIK |
| NO: 359 | EVQLVESGGGLVQPGGSLRLSCAASGFTFSDSWIHWVRQAPGKGLEWVAWISPYGGSTYYADS |
| | VKGRFTISADTSKNTAYLQMNSLRAEDTAVYYCARRHWPGGFDYWGQGTLVTVSS |
| NO: 360 | DIQMTQSPSSLSASVGDRVTITCRASQDVSTAVAWYQQKPGKAPKLLIYSASFLYSGVPSRFSGS |
| | GSGTDFTLTISSLQPEDFATYYCQQYLYHPATFGQGTKVEIK |
| NO: 361 | EVQLLESGGGLVQPGGSLRLSCAASGFTFSSYIMMWVRQAPGKGLEWVSSIYPSGGITFYADTV |
| | KGRFTISRDNSKNTLYLQMNSLRAEDTAVYYCARIKLGTVTTVDYWGQGTLVTVSS |
| NO: 362 | QSALTQPASVSGSPGQSITISCTGTSSDVGGYNYVSWYQQHPGKAPKLMIYDVSNRPSGVSNRFS |
| | GSKSGNTASLTISGLQAEDEADYYCSSYTSSSTRVFGTGTKVTVL |
| NO: 363 | EVQLLESGGGLVQPGGSLRLSCAASGFTFSSYAMSWVRQAPGKGLEWVSAISGSGGSTYYADS |
| | VKGRFTISRDNSKNTLYLQMNSLRAEDTAVYYCAKSPYAPLDYWGQGTLVTVSS |
| NO: 364 | EIVLTQSPATLSLSPGERATLSCRASQSVNDYLAWYQQKPGQAPRLLIYDASNRATGIPARFSGS |
| | GSGTDFTLTISSLEPEDFAVYYCQQGGHAPITFGQGTKVEIK |
| NO: 365 | EVQLVQSGAEVKKPGESLKISCKGSGYSFTSYWMQWVRQMPGKGLEWMGAIYPGDGDIRYTQ |
| | NFKGQVTISADKSISTAYLQWSSLKASDTAMYYCARWEKSTTVVQRNYFDYWGQGTTVTVSS |
| NO: 366 | DIQMTQSPSSLSASVGDRVTITCKASENVGTFVSWYQQKPGKAPKLLIYGASNRYTGVPSRFSGS |
| | GSGTDFTLTISSLQPEDFATYYCGQSYSYPTFGQGTKLEIK |

What is claimed is:

1. A polypeptide that specifically binds a C-X-C motif chemokine receptor 6 (CXCR6), comprising heavy chain complementarity determining regions HCDR1, HCDR2 and HCDR3 that are at least 90% identical to the HCDR1, HCDR2 and HCDR3, respectively, of at least one immunoglobulin heavy chain variable region (V_{H}) set forth in SEQ ID NOs:11-46.

2. The polypeptide of claim 1, further comprising light chain complementarity determining regions LCDR1, LCDR2 and LCDR3 that are at least 90% identical to the LCDR1, LCDR2 and LCDR3, respectively, of at least one immunoglobulin light chain variable region (V_{L}) set forth in SEQ ID NOs:47-82.

3. The polypeptide of claim 2, comprising the HCDR1, HCDR2 and HCDR3, and LCDR1, LCDR2 and LCDR3, of an antibody comprising a V_{H} and a V_{L}, wherein the V_{H} and V_{L} of the antibody are selected from the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:14/SEQ ID NO:50;
SEQ ID NO:15/SEQ ID NO:51;
SEQ ID NO:16/SEQ ID NO:52;
SEQ ID NO:17/SEQ ID NO:53;
SEQ ID NO:18/SEQ ID NO:54;
SEQ ID NO:19/SEQ ID NO:55;
SEQ ID NO:20/SEQ ID NO:56;
SEQ ID NO:21/SEQ ID NO:57;
SEQ ID NO:22/SEQ ID NO:58;
SEQ ID NO:23/SEQ ID NO:59;
SEQ ID NO:24/SEQ ID NO:60;
SEQ ID NO:25/SEQ ID NO:61;
SEQ ID NO:23/SEQ ID NO:62;
SEQ ID NO:26/SEQ ID NO:63;
SEQ ID NO:27/SEQ ID NO:64;
SEQ ID NO:28/SEQ ID NO:65;
SEQ ID NO:29/SEQ ID NO:65;
SEQ ID NO:30/SEQ ID NO:66;
SEQ ID NO:31/SEQ ID NO:67;
SEQ ID NO:32/SEQ ID NO:68;
SEQ ID NO:33/SEQ ID NO:69;
SEQ ID NO:34/SEQ ID NO:70;
SEQ ID NO:35/SEQ ID NO:71;
SEQ ID NO:36/SEQ ID NO:72;
SEQ ID NO:37/SEQ ID NO:73;
SEQ ID NO:38/SEQ ID NO:74;
SEQ ID NO:39/SEQ ID NO:75;
SEQ ID NO:40/SEQ ID NO:76;
SEQ ID NO:41/SEQ ID NO:77;
SEQ ID NO:42/SEQ ID NO:78;
SEQ ID NO:43/SEQ ID NO:69;
SEQ ID NO:44/SEQ ID NO:79;
SEQ ID NO:45/SEQ ID NO:80;
SEQ ID NO:18/SEQ ID NO:81; and
SEQ ID NO:46/SEQ ID NO:82.

4. The polypeptide of claim 3, wherein the V_{H} and V_{L} of the antibody are selected from the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:17/SEQ ID NO:53;
SEQ ID NO:19/SEQ ID NO:55;
SEQ ID NO:20/SEQ ID NO:56; and
SEQ ID NO:21/SEQ ID NO:57.

5. The polypeptide of any one of claims 1-4, wherein the polypeptide is an immunoglobulin molecule that comprises a V_{H} and a V_{L}.

6. The polypeptide of claim 5, wherein:
a) the V_{L} of the polypeptide is at least 85% identical to at least one sequence set forth in SEQ ID NOs:47-82;
b) the V_{H} of the polypeptide is at least 85% identical to at least one sequence set forth in SEQ ID NOs: 11-46; or
c) both b) and c).

7. The polypeptide of claim 6, wherein the polypeptide comprises a V_{H}/V_{L} combination that is identical to any one of the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:14/SEQ ID NO:50;
SEQ ID NO:15/SEQ ID NO:51;
SEQ ID NO:16/SEQ ID NO:52;
SEQ ID NO:17/SEQ ID NO:53;
SEQ ID NO:18/SEQ ID NO:54;
SEQ ID NO:19/SEQ ID NO:55;
SEQ ID NO:20/SEQ ID NO:56;
SEQ ID NO:21/SEQ ID NO:57;
SEQ ID NO:22/SEQ ID NO:58;
SEQ ID NO:23/SEQ ID NO:59;
SEQ ID NO:24/SEQ ID NO:60;
SEQ ID NO:25/SEQ ID NO:61;
SEQ ID NO:23/SEQ ID NO:62;
SEQ ID NO:26/SEQ ID NO:63;
SEQ ID NO:27/SEQ ID NO:64;
SEQ ID NO:28/SEQ ID NO:65;
SEQ ID NO:29/SEQ ID NO:65;
SEQ ID NO:30/SEQ ID NO:66;
SEQ ID NO:31/SEQ ID NO:67;
SEQ ID NO:32/SEQ ID NO:68;
SEQ ID NO:33/SEQ ID NO:69;
SEQ ID NO:34/SEQ ID NO:70;
SEQ ID NO:35/SEQ ID NO:71;
SEQ ID NO:36/SEQ ID NO:72;
SEQ ID NO:37/SEQ ID NO:73;
SEQ ID NO:38/SEQ ID NO:74;
SEQ ID NO:39/SEQ ID NO:75;
SEQ ID NO:40/SEQ ID NO: 76;
SEQ ID NO:41/SEQ ID NO:77;
SEQ ID NO:42/SEQ ID NO:78;
SEQ ID NO:43/SEQ ID NO:69;
SEQ ID NO:44/SEQ ID NO:79;
SEQ ID NO:45/SEQ ID NO:80;
SEQ ID NO:18/SEQ ID NO:81; and
SEQ ID NO:46/SEQ ID NO:82.

8. The polypeptide of claim 7, wherein the polypeptide comprises a V_{H}/V_{L} combination that is identical to any one of the following V_{H}/V_{L} combinations:
SEQ ID NO:11/SEQ ID NO:47;
SEQ ID NO:12/SEQ ID NO:48;
SEQ ID NO:13/SEQ ID NO:49;
SEQ ID NO:17/SEQ ID NO:53;
SEQ ID NO: 19/SEQ ID NO:55;
SEQ ID NO:20/SEQ ID NO:56; and
SEQ ID NO:21/SEQ ID NO:57.

9. The polypeptide of any one of claims 1-8, wherein the polypeptide is an antibody or an antigen-binding fragment thereof.

10. The polypeptide of claim 9, wherein the antigen binding fragment is selected from a Fab, a F(ab′)₂, a Fab′, an scFv, or an Fv.

11. The polypeptide of claim 10, wherein the antigen binding fragment is an scFv.

12. The polypeptide of claim 9, further comprising:
a) an antibody heavy chain constant region sequence,
b) an antibody light chain constant region sequence, or
c) both a) and b).

13. The polypeptide of claim 12 wherein the antibody heavy chain constant region is a human IgG heavy chain constant region.

14. The polypeptide of claim 12 or 13, wherein the antibody heavy chain constant region is an IgG1 heavy chain constant region.

15. The polypeptide of any one of claims 12-14, wherein the antibody light chain constant region is a human constant region.

16. The polypeptide of any one of claims 12-15, wherein the antibody light chain constant region is a k light chain constant region.

17. The polypeptide of any one of claims 1-16, wherein the CXCR6 is a primate CXCR6.

18. The polypeptide of claims 1-17, wherein the primate CXCR6 is a human CXCR6 (SEQ ID NO:1).

19. The polypeptide of claim 17, wherein the primate CXCR6 is a cynomolgus monkey CXCR6 (SEQ ID NO:6).

20. The polypeptide of any one of claims 1-19, wherein the polypeptide specifically binds an epitope within the amino acid sequence set forth in SEQ ID NO:2.

21. The polypeptide of any one of claims 1-19, wherein the polypeptide specifically binds an epitope within the amino acid sequence set forth in SEQ ID NO:3, SEQ ID NO:4, SEQ ID NO:5, SEQ ID NO:8, SEQ ID NO:9, SEQ ID NO: 10, or a combination thereof.

22. The polypeptide of any one of claims 1-21, wherein the polypeptide is conjugated to a heterologous moiety.

23. The polypeptide of claim 22, wherein the heterologous moiety is a therapeutic agent, a diagnostic agent or a combination thereof.

24. A fusion protein comprising the polypeptide of any one of claims 1-23.

25. A polynucleotide comprising a sequence encoding the polypeptide of any one of claims 1-23 or the fusion protein of claim 24.

26. The polynucleotide of claim 25, wherein the polynucleotide is a linear deoxyribonucleic acid (DNA), a linear ribonucleic acid (RNA), a circular DNA or a circular RNA.

27. An expression vector comprising the polynucleotide of claim 25 or 26.

28. A host cell comprising the polynucleotide of claim 25 or 26, or the expression vector of claim 27.

29. A composition comprising the polypeptide of any one of claims 1-23, the fusion protein of claim 24, the polynucleotide of claim 25 or 26, the expression vector of claim 27, or the host cell of claim 28.

30. The composition of claim 29, further comprising one or more of a pharmaceutical excipient, a diluent, or a carrier.

31. A method of modulating a CXCR6-positive leukocyte, comprising contacting the CXCR6-positive leukocyte with the composition of claim 29 or 30.

32. A method of blocking chemotaxis of a CXCR6-positive leukocyte, comprising contacting the CXCR6-positive leukocyte with the composition of claim 29 or 30.

33. A method of inactivating a CXCR6-positive leukocyte, comprising contacting the CXCR6-positive leukocyte with the composition of claim 29 or 30.

34. A method of killing a CXCR6-positive leukocyte, comprising contacting the CXCR6-positive leukocyte with the composition of claim 29 or 30.

35. The method of any one of claims 31-34, wherein the CXCR6-positive leukocyte is a CD4 T cell, a CD8 T cell, a γδ T cell, a natural killer (NK) T cell, an NK cell or a neutrophil.

36. The method of any one of claims 31-35, comprising contacting the CXCR6-positive leukocyte with the composition of claim 29 or 30 for about 1 minute to about 10 days, wherein the composition comprises about 0.05-20 μg/ml of the polypeptide of any one of claims 1-23.

37. The method of any one of claims 34-36, wherein the method kills the CXCR6-positive leukocyte by antibody-dependent cellular cytotoxicity (ADCC), antibody-dependent cellular phagocytosis (ADCP), complement-dependent cytotoxicity (CDC), or a combination thereof.

38. A method of activating a CXCR6-positive leukocyte, comprising contacting the CXCR6-positive leukocyte with the composition of claim 29 or 30.

39. A method of maintaining the survival of a CXCR6-positive leukocyte, comprising contacting the CXCR6-positive leukocyte with the composition of claim 29 or 30.

40. The method of claim 38 or 39, wherein the CXCR6-positive leukocyte is a CD4 T cell, a CD8 T cell, a γδ T cell, an NK T cell, an NK cell or a neutrophil.

41. The method of any one of claims 38-40, comprising contacting the CXCR6-positive leukocyte with the composition of claim 29 or 30 for about 1 minute to about 10 days, wherein the composition comprises about 0.05-20 μg/ml of the polypeptide of any one of claims 1-23.

42. A method of depleting CXCR6-positive leukocytes in a subject in need thereof, comprising administering an effective amount of the composition of claim 29 or 30 to the subject.

43. A method of treating or preventing a disease in a subject in need thereof, comprising administering an effective amount of the composition of claim 29 or 30 to the subject.

44. The method of claim 42 or 43, wherein the subject has or is at risk of developing acute Graft versus Host Disease (aGvHD), allograft rejection, asthma, autoimmune hepatitis, autoimmune uveitis, contact dermatitis, Crohn's Disease, juvenile rheumatoid arthritis, multiple sclerosis, nonalcoholic steatohepatitis, psoriasis, psoriatic arthritis, rheumatoid arthritis, systemic lupus erythematosus, uveitis or ulcerative colitis.

45. The method of claim 44, wherein the subject has or is at risk of developing aGvHD, multiple sclerosis or rheumatoid arthritis.

46. The method of any one of claims 42-44, wherein the subject is a human.

47. The method of any one of claims 42-46, wherein the composition of claim 29 or 30 is administered intravenously.

48. The method of any one of claims 42-46, wherein the composition of claim 29 or 30 is administered subcutaneously.

49. The method of any one of claims 42-48, further comprising administering a therapeutically effective amount of an additional therapeutic agent to the subject.

50. The method of claim 49, wherein the additional therapeutic agent is a monoclonal antibody, a steroid, an immunosuppressant, or an agent targeting an IL-23/IL-17 axis.

51. The method of claim 50, wherein the additional therapeutic agent is a monoclonal antibody that specifically binds CD20, IL-6, IL-1β, IL-6 receptor, IL-12, IL-23p40, TNFα, or a combination thereof.

52. The method of claim 50, wherein the additional therapeutic agent is prednisone.

53. The method of claim 50, wherein the additional therapeutic agent is cyclophosphamide, fingolimod or methotrexate.

54. The method of claim 50, wherein the additional therapeutic agent is a Janus kinase (JAK) inhibitor.

55. The method of claim 54, wherein the JAK inhibitor is Ruxolitinib (INCB018424), Momelotinib, fedratinib (TG101348) or tofacitinib.

56. A method of activating CXCR6-positive cells in a subject in need thereof, comprising administering an effective amount of the composition of claim 29 or 30 to the subject.

57. The method of claim 56, wherein the subject has or is at risk of developing cancer.

58. The method of claim 57, wherein the cancer is breast cancer, colon cancer, gastric cancer, liver cancer, lung cancer, lymphoma, melanoma, non-small cell lung cancer or oesophageal cancer.

59. The method of claim 58, wherein the cancer is colon cancer.

60. The method of any one of claims 56-59, wherein the subject is a human.

61. The method of any one of claims 56-60, wherein the composition of claim 29 or 30 is administered intravenously.

62. The method of any one of claims 56-60, wherein the composition of claim 29 or 30 is administered subcutaneously.

63. The method of any one of claims 56-62, further comprising administering a therapeutically effective amount of an additional therapeutic agent to the subject.

64. The method of claim 63, wherein the additional therapeutic agent is a chemotherapeutic agent or an immune checkpoint inhibitor.

65. The method of claim 64, wherein the immune checkpoint inhibitor is an anti-PD-1 antibody or recombinant fusion protein, an anti-PD-L1 antibody or probody, an anti-PD-L2 antibody, an anti-LAG-3 antibody, an anti-TIM-3 antibody or an anti-CTLA-4 antibody.


SEQUENCE LISTING (366 sequences)

SEQ ID NO: 1 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 2 - protein, 32 aa, Homo sapiens
MAEHDYHEDYGFSSFNDSSQEEHQDFLQFSKV

SEQ ID NO: 3 - protein, 15 aa, Homo sapiens
AGIHEWVFGQVMCKS

SEQ ID NO: 4 - protein, 23 aa, Homo sapiens
PQIIYGNVFNLDKLICGYHDEAI

SEQ ID NO: 5 - protein, 16 aa, Homo sapiens
YYAMTSFHYTIMVTEA

SEQ ID NO: 6 - protein, 343 aa, Macaca fascicularis
MAEYDHYEDDGFLNSFNDSSQEEHQDFLQFRKVFLPCMYLVVFVCGLVGNSLVLVISIFYHKLQSLTDVFLVNLPLADLVFVCTLPFWTYAGIHEWIFGQVMCKTLLGVYTINFYTSMLILTCITVDRFIVVVKATKAYNQQAKRMTWGKVICLLIWVISLLVSLPQIIYGNVFNLDKLICGYHDEEISTVVLATQMTLGFFLPLLAMIVCYSVIIKTLLHAGGFQKHRSLKIIFLVMAVFLLTQTPFNLVKLIRSTRWEYYAMTSFHYTIIVTEAIAYLRACLNPVLYAFVSLKFRKNFWKLVKDIGCLPYLGVSHQWKSSEDNSKTFSASHNVEATSMFQL

SEQ ID NO: 7 - protein, 33 aa, Macaca fascicularis
MAEYDHYEDDGFLNSFNDSSQEEHQDFLQFRKV

SEQ ID NO: 8 - protein, 14 aa, Macaca fascicularis
AGIHEWIFGQVMCK

SEQ ID NO: 9 - protein, 23 aa, Macaca fascicularis
PQIIYGNVFNLDKLICGYHDEEI

SEQ ID NO: 10 - protein, 16 aa, Macaca fascicularis
YYAMTSFHYTIIVTEA

SEQ ID NO: 11 - protein, 121 aa, Artificial Sequence
  VH Genovac2-C1
DVKLVESGGGLVKPGGSLKLSCAASGFTFSSYTMSWVRQTPEKRLEWVATISSGGSYTYYPDSVKGRFTISRDNAKNTLYLQMSSLKSEDTAMYYCTRGGGNYPFWFFDVWGAGTTVTVSS

SEQ ID NO: 12 - protein, 118 aa, Artificial Sequence
  VH Genovac1-G2
DVQLQESGPGLVKPSQSLSLTCSVTGYSITSGYYWNWIRQFPGNKLEWMGYIRYDGSNNYNPSLKNRISITRDTSKNQFFLKLNSVTTEDTATYYCARDGYSTYFDYWGQGTTLTVSS

SEQ ID NO: 13 - protein, 119 aa, Artificial Sequence
  VH Beacon2-G5
EVQLVESGGDLVKPGGSLKLSCAASGFTFSSYGMSWVRQTPDKRLEWVATISSRGSYTYYPDSVKGRLTISRENAKNTLSLQMSSLKSEDTAMYYCARRVFPYWFFDVWGAGTTVTVSS

SEQ ID NO: 14 - protein, 121 aa, Artificial Sequence
  VH Beacon2-F1
EVQLVESGGDLVKPGGSLKLSCAASGFTFSSYGMSWVRQTPDKRLEWVATISSGGSYTYYPDSVKGRFPISRDNAKNTLYQQMSSLNSEDTAMYYCARLYGNYPYFAMDYWGQGTSVTVSS

SEQ ID NO: 15 - protein, 120 aa, Artificial Sequence
  VH CNE-6E5-C1
QVTLKESGPGILQSSQILSLTCSFSGFSLITSGMGVSWIRKTSGKGLEWLAHIYWDDEKRYNPSLKSRLTISKGTSRNQVFLRITSVDTTDTATYFCVRREDFGDGFDYWGQGTTLTVSP

SEQ ID NO: 16 - protein, 119 aa, Artificial Sequence
  VH CNGs-3B2
DVQLVESGGGLVQPGGSRTLSCAASGFTFSSFGMHWVRQAPEKGLEWVAYISSGSTTIYYADTVKGRFTISRDNPKNTLFLQVTSLRSEDTAVYYCTRSHWPYWYFDVWGAGTTVTVSS

SEQ ID NO: 17 - protein, 120 aa, Artificial Sequence
  VH Beacon1-B2
  misc_feature (9)..(9): Xaa can be any naturally occurring amino acid
EVQLQQSGXELVKPGASVKISCKASGYTFTDYYMNWVKLYHGKSLQWIGDINPNNGGTTYNQKFKGKATLTVDKSSSTAYMELRSLTSEDSAVYYCAKSRRNWGYAMDYWGQGTSVTVSS

SEQ ID NO: 18 - protein, 117 aa, Artificial Sequence
  VH CNGs-1B7, Genovac2-H2
EVKLEESGGGLVRPGGSMKLSCVASGFTFSDYWMNWVRQSPEKGLEWLAQIRLNSGNYAARYAESVKGRFTISRDDSKNSVYLQMINLRAEDTGIYFCTSNAWLANWGQGTLVTVSA

SEQ ID NO: 19 - protein, 117 aa, Artificial Sequence
  VH CNE-6E10
EVQLVESGGGLVQPKGSLKLSCAASGFSFNTYAMNWVRQAPGKGLEWVARIRSKTNSYETYYADSVKDRFTISRDDSESMLYLQMNNLKTEDTAVYYCLSHGAFAYWGQGTLVTVSA

SEQ ID NO: 20 - protein, 118 aa, Artificial Sequence
  VH COXs-1B10
QVTLKESGPGILKASQTLSLTCSFSGFSLSTSGMGIGWIRQPSGKGPEWLAHIWWDDEKYYRPSLKSRLTVSKDTSRNQVFLNITSVDTTDSASYYCVQRTPGRFAYWGQGTLVTVST

SEQ ID NO: 21 - protein, 117 aa, Artificial Sequence
  VH COXs-2D2
EVQLVESGGGLVQPKGSLKLSCTASGFSFDTYAMNWVRQAPGKGLEWIARIRSKSKNYATYYADSVKDRFTISRDDSANILYLQMNNLKTEDTAIYYCLRQGSFDYWGQGTTLTVSS

SEQ ID NO: 22 - protein, 114 aa, Artificial Sequence
  VH Beacon1-D10
EVQLMESGGGLVKPGGSRKLSCAASGFTFSDHGMHWVRQAPEKGLEWIAYINSGSSTIHYADSVKGRFTISRDNAKNTLFLQMTSLRSEDTAMYYCVRPGFAYWGQGTLITVSA

SEQ ID NO: 23 - protein, 120 aa, Artificial Sequence
  VH Beacon1-G12, COXs-4H5
EIQLVQSGPELKKPGKTVKISCKASGYTFTTYGMSWVKQAPGKALKWMGWINTYSGVPTYADDFKGRFAFSLETSASTAYLQINNLKNEDTATYFCAIRRLSITLHGMDYWGQGTSVTVS

SEQ ID NO: 24 - protein, 117 aa, Artificial Sequence
  VH COXs-1B11
EVQLLEFGGGLVQPKGSLKLSCAASGFTFNTYAMNWVRQVQGKGLEWIARVRSKVKNYATYYADSVKDRFTISRDDSESILYLQMNNLKTEDTGIYYCVRQGNFDVWGTGTSVTVSS

SEQ ID NO: 25 - protein, 117 aa, Artificial Sequence
  VH COXs-2D4
EVHLVESGGGLVQPKGSLKLSCTASGFSFDTYAMNWVRQAPGKGLEWIARIRSKSKNSATYYADSVKDRFTISRDDSANILYLQMNNLKTEDTAIYYCLRQGSFDYWGQGTTLTVSS

SEQ ID NO: 26 - protein, 114 aa, Artificial Sequence
  VH COXs-7A7
EVQLMESGGGLVKPGGSRKLSCAASGFTFSDYGMHWVRQAPEKGLEWIAYINSGSSTIHYADSVKGRFTISRDNAKNTLFLQMTSLRSEDTAMYYCVRPGFAYWGQGTLITVSA

SEQ ID NO: 27 - protein, 114 aa, Artificial Sequence
  VH COXs-7E6
EVQLMESGGGLVKPGGSRKLSCAASGFTFSDHGMHWVRQAPEKGLEWIAYINSGSSTIHYADSVKGRFTISRDNAKNTLFLHMTSLRSEDTAMYYCVRPGFAYWGQGTLITVSA

SEQ ID NO: 28 - protein, 117 aa, Artificial Sequence
  VH COXs-9D6
EVHLVESGGGLVQPKGSLKLSCTASGFSFDTYAMNWVRQAPGKGLEWVARIRSKSKNYATYYADSVTDRFTISRDDSANILYLQMNNLKTEDTAIYYCLSQGSFDYWGQGTTLTVSS

SEQ ID NO: 29 - protein, 117 aa, Artificial Sequence
  VH COXs-11E5
EVQLVESGGGLVQPKGSLKLSCTASGFSFDTYAMNWVRQAPGKGLEWVARIRSKSKNYATYYADSVKDRFTVSRDDSANILYLQMNNLKTEDTAIYYCLRQGSFDYWGQGTTLTVSS

SEQ ID NO: 30 - protein, 117 aa, Artificial Sequence
  VH COXs-11H4
EVQLVESGGGLVQPKGSLKLSCAASGFTFNTYAMNWVRQVPGKGLEWIARMRSESKNFATYYADSVKDRFTISRDDSESILYLQMNNLKTEDTGMYYCVRQGNFDVWGTGTTVTVSS

SEQ ID NO: 31 - protein, 117 aa, Artificial Sequence
  VH COXs-12E7
EVHLVESGGGLVQPKGSLKLSCKASGFSFDTYAMNWVRQAPGKGLEWVARIRSKSKSYATYYADSVKDRFTISRDDSANILYLQMNNLKTEDTAIYYCLRQGSFDYWGQGTTLTVSS

SEQ ID NO: 32 - protein, 116 aa, Artificial Sequence
  VH CNE-7B1
QVQLQQSGPELVKPGTSVRISCKTSGYTFTSSYVHWVKRRPGQGLEWIGWIYPGNDYTKYNEKFKGKATLTADKSSSTAYMRLSSLASEDSAVYFCTRSYYGFDYWGQGTTLTVSS

SEQ ID NO: 33 - protein, 117 aa, Artificial Sequence
  VH CNF-2F8
EVQLVESGGGLVQPKGSLKLSCAASGFSFQTYAMNWVRQAPGKGLEWVARIRSKRNNYATYYADSVKDRFSISRDDSENMLYLQMNNLKTEDTAMYYCLSHGAFAYWGQGTLVTVSA

SEQ ID NO: 34 - protein, 117 aa, Artificial Sequence
  VH CNGs-1C1
EVQLVESGGGLVRPKGSLKLSCAASGFSFNAYAMNWVRQAPGKGLEWVARIRSKSNNYATYYADSVKDRFTISRDDSESMLYLQMNNLKTEDTAMYYCLSHGAFAYWGQGTLVTVSA

SEQ ID NO: 35 - protein, 113 aa, Artificial Sequence
  VH CNGs-2F11
EVQLQQSGPVLVKPGASVRMSCKASGYTFTEYYINWVKQGHGKSLEWIGIFNPYNGNTKYNQNFKGKATLTVDMSSSTAYMELSSLTSEDSAVYYCSRPYSNFGQGTLVTVSA

SEQ ID NO: 36 - protein, 117 aa, Artificial Sequence
  VH CNE-6H7-G7
EVQLVESGGGLVQPKGSLKLSCAASGFSFNTYAMNWVRQAPGKGLEWVARIRSKSNNYGTYYADSVKDRFTISRDDSESMLYLKMNNLKTEDTAMYYCLSHGSSAYWGQGTLVTVSA

SEQ ID NO: 37 - protein, 115 aa, Artificial Sequence
  VH CNGs-1B7-E4
EVQLVESGGGLVQPRGSLKLSCAASGFTFNTYAMNWVRQAPGNGLEWVARIRSKSYNYATYHAGSVKDRFTISRDDSQNMLYLQMNNLKTEDTAMYYCMRGQTYWGQGTLVTVSA

SEQ ID NO: 38 - protein, 116 aa, Artificial Sequence
  VH CNGs-1E1-G5
QVQLQQSGPELVKPGASVRISCKASGYTFTTSYIHWVKQRPGQGLEWIGWISPGNVNTRSNEKFKGKATLTADKSSNTAYMQLSSLTSEDSAVYFCAKSYFGFDFWGQGTTLTVSS

SEQ ID NO: 39 - protein, 113 aa, Artificial Sequence
  VH CNGs-3G6-G5
EVQLQQSGPVLVKPGSSVKLSCKASGYTFTEYYINWVKQSHGKRLEWIGIFNPYNGNTKYNQKFKGKATLTVDKSSSTAYMELSSLTSEDSAVYYCSRPYSNFGQGTLVTVAA

SEQ ID NO: 40 - protein, 115 aa, Artificial Sequence
  VH CNGs-3H2-B7
EVQLVESGGGLVQPKGSLKLSCAASGVSFKTYAMTWVRQAPGKGLEWVARIRSKRYNYATYYADSVKDRFTISRDDSESMLYLQMNNLKTEDTAMYYCMRQGAYWGQGTLVTVSA

SEQ ID NO: 41 - protein, 115 aa, Artificial Sequence
  VH Beacon2-A3
EVQIVESGGGLVQPKGSLKLSCAASGFSFNTYAMNWVRQAPGKGLEWVARIRTKRNNYVKFYADSVKDRFTISRDDSESMLYLQMNILKTEDTAMYYCVRQGAYWGQGTLVTVSA

SEQ ID NO: 42 - protein, 117 aa, Artificial Sequence
  VH Beacon2-A7
EMKLEESGGGLVQPGGSMKLSCVASGFTFSNYWMSWVRQSPEKGLQWVGQIRLKSDNYATHYAESVKGKFTISRDDSKSSVYLQMNNLRAEDTGIYYCAPYSNWGYWGQGTTLTVSS

SEQ ID NO: 43 - protein, 117 aa, Artificial Sequence
  VH Beacon2-D5
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCLSHGAFAYWGQGTLVTVSA

SEQ ID NO: 44 - protein, 115 aa, Artificial Sequence
  VH Beacon2-E5
EVQLVESGGGLVQPKGSLKLSCAASGFSFNTYAMNWVRQAPGKGLEWVARIRTKRNNYVKFYADSVKGRFTISRDDSQSMLYLQMNNLKAEDTAIYYCVRQGAFWGQGTLVTVSA

SEQ ID NO: 45 - protein, 117 aa, Artificial Sequence
  VH Genovac1-D12
EVKLEESGGGLVQPGGSMKLSCVASGFTFSNYWMNWVRQSPEKGLKWVAQIRLKSDNYGTHYAESVKGRFTISRDDSKGSVYLQMNNLRAEDTGIYYCTSNAWFAYWGQGTLVTVSA

SEQ ID NO: 46 - protein, 128 aa, Artificial Sequence
  VH Beacon1-A4
EVQLQQSGPVLVKPGASVKMSCKTSGYTFTDYYMNWVKQSHGKSLEWIGVIKPYSGRTSYDQKFKDKATLTVDKSSSTAYMELNSLTSEDSAVYYCAREGVSITTVGSSLYYYPMDYWGQGTSVTVSS

SEQ ID NO: 47 - protein, 107 aa, Artificial Sequence
  VL Genovac2-C1
DIQMTQSPASLSASVGETVTITCRASENIYSYLAWYQQKQGKSPQLLVYNAKTLAEGVPSRFSGSGSGTQFSLKIKSLQPEDFGSYYCQHHYGIPRTFGGGTKVETK

SEQ ID NO: 48 - protein, 107 aa, Artificial Sequence
  VL Genovac1-G2
DIQMTQSPASLSASVGETVTITCRASENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 49 - protein, 107 aa, Artificial Sequence
  VL Beacon2-G5
DIVMTQSHKFMSTSVGDRVSITCKASQDVSTAVAWYQQKSGQSPKLLIYSASYRYTGVPDRFTGSGSGTDFTFTISSVQAEDLAVYYCQQHYTTPYTFGGGTKLEIK

SEQ ID NO: 50 - protein, 107 aa, Artificial Sequence
  VL Beacon2-F1
DIVMTQSQKFMSTSVGDRVSITCKASQNVGTAVAWYQQKPGQSPKLLIFSASNRYTGVPDRFTGSGSGTDFTLTISNMQSEDLADYFCQQYSSYPYTFGGGTKLEIK

SEQ ID NO: 51 - protein, 112 aa, Artificial Sequence
  VL CNE-6E5-C1
DVLMTQAPLSLPVSLGDQASISCRSSQTIVHSNGVTYLEWYLQKPGQSPKLLIYKVSNRFSGVPDRFSGSGSGTYFTLKISRVEAEDLGVYYCFQGSHVPPTFGGGTKLEIK

SEQ ID NO: 52 - protein, 107 aa, Artificial Sequence
  VL CNGs-3B2
DVQITQSPSYLAASPGETITINCRASKSISKYLAWYQEKPGKTNKLLIYSGSTLQSGNPSRFSGSGSGTDFTLTISRLEPEDCAMYYCQQHNEFPWTFGGGTKLEIK

SEQ ID NO: 53 - protein, 110 aa, Artificial Sequence
  VL Beacon1-B2
  misc_feature (2)..(2): Xaa can be any naturally occurring amino acid
DXVLTQSPASLAVSLGQRATISCRASESVSVRGTGLMHWYQQKPGYPPKLLIYAASNLESGVPARFSGRRSGTDFTLNIHPVEEDDAATYYCQQSREYRTFGGGTKLEIK

SEQ ID NO: 54 - protein, 112 aa, Artificial Sequence
  VL CNGs-1B7
DIVMTQTAPSVPVTPGESVSISCRSSKSLLHSNGNTYLYWFLQRPGQSPQRLIYSVSNLASGVPDRFSGRGSGTDFTLTISRVEAEDVGVYYCMQSLEYPYTFGGGTKLEIK

SEQ ID NO: 55 - protein, 112 aa, Artificial Sequence
  VL CNE-6E10
DVVMTQTPLTLSVTIGQPASISCKSSQSLLDINGKTYLNWLLQRPGQSPKRLIYLVSKLDSGVPDRFTGSGSGTDFTLKISRVEAEDLGVYYCWQGTHFPQTFGGGTKLEIK

SEQ ID NO: 56 - protein, 113 aa, Artificial Sequence
  VL COXs-1B10
DIVMTQSPSSLAVSIGQKVIMSCKSSQSLLSSRTQKNYLAWYQQKPGQSPKLLVYFASIRESGVPDRFTGSGSGTDFTLTISTVQAEDLAVYYCQQYFLTPYTFGGGTKLEIK

SEQ ID NO: 57 - protein, 112 aa, Artificial Sequence
  VL COXs-2D2
DVVLTQTPLSLPVSLGDQASISCRSGQRIVHTDGNTYLYWYLQKPGQSPKLLIYRVSNRFSGVPDRFSGSGSGTDFTLKISRVEAEDMGIYYCFQGTHFPYTFGGGTKLEIK

SEQ ID NO: 58 - protein, 112 aa, Artificial Sequence
  VL Beacon1-D10
RRDDPRLHSPCLTVLEIQPPSSSDLIRTLCTMMETRIYIGPCRNQCQSPQLLIYRVSNRYSGVPDRFSGSGSGTDFTLKITRVEAEDMGVYYCFQATHDPFTFGGGTKLEIK

SEQ ID NO: 59 - protein, 107 aa, Artificial Sequence
  VL Beacon1-G12
DIQMTQSPASLSVSVGETVTITCRASENIYSNLAWYQQKQGKSPQLLVYGATNLADGVPSRFSGSGSGTQYSLKINSLQSEDFGSYYCQHFWGTPWTFGGGTKLEIK

SEQ ID NO: 60 - protein, 112 aa, Artificial Sequence
  VL COXs-1B11
DVVLTQTPLSLSVSLGDQASISCRSGQNIVHTDGNTYLYWSLQKPGQSPGLLIYKISNRFSGVPDRFSGSGSGTDFTLKISRVEAEDVGVYYCFQLTHFPYTFGGGTKLEIK

SEQ ID NO: 61 - protein, 112 aa, Artificial Sequence
  VL COXs-2D4
DVVMTQTPLSLPVSLGDQASISCRSSQRIVHTDGNTYLYWYLQKPGQSPKLLIYRVSNRFSGVPDRFSGSGSGTDFTLKITRVEAEDMGIYYCFQGTHFPYTFGGGTKLEIK

SEQ ID NO: 62 - protein, 112 aa, Artificial Sequence
  VL COXs-4H5
DVVMTQSPLSLPVSLGDQASIYCRSSQRIVHSDGNTYLYWYLQRPGQSPKLLIYRVSNRFSGVPDRFSGSGSGTDFTLKISRVEAEDMGIYYCFQGTHFPYTFGGGTKLEIK

SEQ ID NO: 63 - protein, 112 aa, Artificial Sequence
  VL COXs-7A7
DIVMTQTPLTLSTTVGQSAFISCRSSQSLLHRNGNTYLNWFLQRPGQSPQLLVYEIFERKSGVPDRFSGSGSGTDFTLKISRVEAEDLGVYYCMQATYEPLTFGAGTKLELK

SEQ ID NO: 64 - protein, 112 aa, Artificial Sequence
  VL COXs-7E6
DIVMTQTPLTLSITVGQSAFISCRSSQSLLHRNGNTYLNWFLQRPGQSPQLLVYEIFERKSGVPDRFSGSGSGTDFTLKISRVEAEDLGVYYCMQATYEPLTFGAGTKLELK

SEQ ID NO: 65 - protein, 112 aa, Artificial Sequence
  VL COXs-9D6, COXs-11E5
DVVMTQTPLSLPVSLGDQASISCRSSQRIVHTDGNTYLYWYLQKPGQSPKLLIYRVSNRFSGVPDRFSGSGSGTDFILKISRVEAEDMGIYYCFQGTHFPYTFGGGTKLEIK

SEQ ID NO: 66 - protein, 112 aa, Artificial Sequence
  VL COXs-11H4
DVVMTQAPLSLSVSLGDQASISCRSGQNIVHTDGNTYLYWSLQKPGQSPNLLIYKVSNRFSGVPDRFSGSGSGTDFTLKINRVEAEDMGVYYCFQLTHVPYTFGGGTKLEIK

SEQ ID NO: 67 - protein, 112 aa, Artificial Sequence
  VL COXs-12E7
DVVMTQTPLSLPVSLGDQASIYCRSSQRIVHSDGNTYLYWYLQRPGQSPKLLIYRVSNRFSGVPDRFSGSGSGTDFTLKISRVEAEDMGIYYCFQGTHFPYTFGGGTKLEIK

SEQ ID NO: 68 - protein, 112 aa, Artificial Sequence
  VL CNE-7B1
DVLMTQTPLSLPVSLGDQASISCRSGQSIVRSRGNTYLEWYLQKPGQSPKLLIYKVSNRFSGVPDRFSGSGSGTDFTLKISRVEAEDLGVYYCFQGSHVPYTFGGGTKLEIK

SEQ ID NO: 69 - protein, 112 aa, Artificial Sequence
  VL CNF-2F8, Beacon2-D5
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 70 - protein, 112 aa, Artificial Sequence
  VL CNGs-1C1
DVVMTQTPLTLSVTIGQPASISCKSSQSLLYSDGKTYLNWLLQRPGQSPKRLIYLVSKLDSGVPDRFTGSGSGTDFTLKISRVEAEDLGVYYCWQATHFPQTFGGGTKLEIK

SEQ ID NO: 71 - protein, 112 aa, Artificial Sequence
  VL CNGs-2F11
DVLLTQIPLSLPVSLGDQASISCRSSQSIVHSNGNTYLEWYLQKPGQSPKLLIYKVSNRFSGVPDRFSGSGSGTDFTLKISRVGAEDLGVYYCFQGSHVPFTFGSGTKLEIK

SEQ ID NO: 72 - protein, 112 aa, Artificial Sequence
  VL CNE-6H7-G7
DVVLIQTPLTLSVTIGQPASISCRSSQSLLYSDGNTYLNWLLQRPGQSPKRLIYLVSELDSGVPDRFTGSGSGTDFTLKISRVEAEDLGVYYCWQGTHFPQTFGGGTKLEIK

SEQ ID NO: 73 - protein, 112 aa, Artificial Sequence
  VL CNGs-1B7-E4
DVVLTQTPLSLPVNIGDQASISCKSTKSLLNSDGFTYLDWHLQKPGQSPQLLIYLVSNRFSGVPDRFSGSGSGTDFTLKISRVEAEDLGVYYCFQSNYLPFTFGSGTKLEMK

SEQ ID NO: 74 - protein, 112 aa, Artificial Sequence
  VL CNGs-1E1-G5
DVLMTQTPLSLPVSLGDQASISCRSSQSIVHSNGNTYLEWYVQKPGQSPKLLIYKVSNRSSGVPDRFSGSGSGTDFTLKISRVEAEDLGVYYCFQGSHVPYTFGGGTKLEIK

SEQ ID NO: 75 - protein, 112 aa, Artificial Sequence
  VL CNGs-3G6-G5
DVLMTQTPLSLPVSLGDQASISCRSSQSLVHSNGNTFLEWFLQKPGQSPKLLIYKVSNRFSGVPDRFSGSGSGTDFTLKISRVEAEDLGVYYCFQGSHVPFTFGSGTKLEIK

SEQ ID NO: 76 - protein, 112 aa, Artificial Sequence
  VL CNGs-3H2-B7
DVVLTQTPLSLPVNIGDQASISCKSTKSLLNSDGFTYLDWYLQKPGQSPHLLIYLVSNRFSGVPDRFSGSGSGTDFTLKISRVEAEDLGVYYCFQSNYLPFTFGSGTKLEIK

SEQ ID NO: 77 - protein, 112 aa, Artificial Sequence
  VL Beacon2-A3
DVVLTQTPLSLPVNIGDQASISCKSTKSLLNSDGFTYLDWYLQKPGQSPQLLIYLVSNRFSGVPDRFSGSGSGTDFTLKISRVEAEDLGVYFCFQSNYLPYTFGGGTKLEIK

SEQ ID NO: 78 - protein, 112 aa, Artificial Sequence
  VL Beacon2-A7
DIVMTQAAPSIPVTPGGSVSISCRSTKSLLHTNGNTYLYWFLQRPGQSPQVLIYRMSNLASGVPDRFSGSGSGTAFTLRISRVEAEDVGVYYCMQYLEYPYTFGSGTKLEIK

SEQ ID NO: 79 - protein, 112 aa, Artificial Sequence
  VL Beacon2-E5
DVVLTQTPLSLPVNIGDQASISCKSTKSLLNSDGFTYLDWYLQKPGQSPQLLIYLVSNRFSGVPDRFSGSGSGTDFTLRISRVEAEDLGVYYCFQSNYLPYTFGGGTKLEIK

SEQ ID NO: 80 - protein, 112 aa, Artificial Sequence
  VL Genovac1-D12
DIVMTQAAPSVPVTPGESVSISCRSSKSLLHSNGNTYLFWFLQRPGQSPQRLISYMSNLASGVPDRFSGRGSGTDFTLRISRVEAEDVGVYYCLLSLEYPYTFGSGTRLEIT

SEQ ID NO: 81 - protein, 107 aa, Artificial Sequence
  VL Genovac2-H2
DIQMTQSPASLSVSVGETVTITCRASENIYNNLAWYQQKQGKSPQLLVYAATNLADGVPSRFSGSGSGTQYSLKINSLQSEDFGSYYCQHFWGTPWTFGGGTKLEIK

SEQ ID NO: 82 - protein, 107 aa, Artificial Sequence
  VL Beacon1-A4
DIVLTQSPATLSVTPGASVSLSCRASQSISNNLHWYQQKSHESLRLLIKYASQSVSGIPSRFSGSGSGTDFTLTINSVETEDFGMYFCQQSNSWPRTFGGGTKLEIK

SEQ ID NO: 83 - protein, 8 aa, Artificial Sequence
  HCDR1 Genovac2-C1
GFTFSSYT

SEQ ID NO: 84 - protein, 8 aa, Artificial Sequence
  HCDR2 Genovac2-C1
ISSGGSYT

SEQ ID NO: 85 - protein, 14 aa, Artificial Sequence
  HCDR3 Genovac2-C1
TRGGGNYPFWFFDV

SEQ ID NO: 86 - protein, 9 aa, Artificial Sequence
  HCDR 1 Genovac1-G2
GYSITSGYY

SEQ ID NO: 87 - protein, 7 aa, Artificial Sequence
  HCDR2 Genovac1-G2
IRYDGSN

SEQ ID NO: 88 - protein, 11 aa, Artificial Sequence
  HCDR3 Genovac1-G2
ARDGYSTYFDY

SEQ ID NO: 89 - protein, 8 aa, Artificial Sequence
  HCDR1 Beacon2-G5
GFTFSSYG

SEQ ID NO: 90 - protein, 8 aa, Artificial Sequence
  HCDR2 Beacon2-G5
ISSRGSYT

SEQ ID NO: 91 - protein, 12 aa, Artificial Sequence
  HCDR3 Beacon2-G5
ARRVFPYWFFDV

SEQ ID NO: 92 - protein, 14 aa, Artificial Sequence
  HCDR3 Beacon2-F1
ARLYGNYPYFAMDY

SEQ ID NO: 93 - protein, 10 aa, Artificial Sequence
  HCDR1 CNE-6E5-C1
GFSLITSGMG

SEQ ID NO: 94 - protein, 7 aa, Artificial Sequence
  HCDR2 CNE-6E5-C1
IYWDDEK

SEQ ID NO: 95 - protein, 12 aa, Artificial Sequence
  HCDR3 CNE-6E5-C1
VRREDFGDGFDY

SEQ ID NO: 96 - protein, 8 aa, Artificial Sequence
  HCDR1 CNGs-3B2
GFTFSSFG

SEQ ID NO: 97 - protein, 8 aa, Artificial Sequence
  HCDR2 CNGs-3B2
ISSGSTTI

SEQ ID NO: 98 - protein, 12 aa, Artificial Sequence
  HCDR3 CNGs-3B2
TRSHWPYWYFDV

SEQ ID NO: 99 - protein, 8 aa, Artificial Sequence
  HCDR1 Beacon1-B2
GYTFTDYY

SEQ ID NO: 100 - protein, 8 aa, Artificial Sequence
  HCDR2 Beacon1-B2
INPNNGGT

SEQ ID NO: 101 - protein, 13 aa, Artificial Sequence
  HCDR3 Beacon1-B2
AKSRRNWGYAMDY

SEQ ID NO: 102 - protein, 8 aa, Artificial Sequence
  HCDR1 CNGs-1B7
GFTFSDYW

SEQ ID NO: 103 - protein, 10 aa, Artificial Sequence
  HCDR2 CNGs-1B7
IRLNSGNYAA

SEQ ID NO: 104 - protein, 8 aa, Artificial Sequence
  HCDR3 CNGs-1B7
TSNAWLAN

SEQ ID NO: 105 - protein, 8 aa, Artificial Sequence
  HCDR1 CNE-6E10
GFSFNTYA

SEQ ID NO: 106 - protein, 10 aa, Artificial Sequence
  HCDR2 CNE-6E10
IRSKTNSYET

SEQ ID NO: 107 - protein, 8 aa, Artificial Sequence
  HCDR3 CNE-6E10
LSHGAFAY

SEQ ID NO: 108 - protein, 10 aa, Artificial Sequence
  HCDR1 COXs-1B10
GFSLSTSGMG

SEQ ID NO: 109 - protein, 7 aa, Artificial Sequence
  HCDR2 COXs-1B10
IWWDDEK

SEQ ID NO: 110 - protein, 10 aa, Artificial Sequence
  HCDR3 COXs-1B10
VQRTPGRFAY

SEQ ID NO: 111 - protein, 8 aa, Artificial Sequence
  HCDR1 COXs-2D2
GFSFDTYA

SEQ ID NO: 112 - protein, 10 aa, Artificial Sequence
  HCDR2 COXs-2D2
IRSKSKNYAT

SEQ ID NO: 113 - protein, 8 aa, Artificial Sequence
  HCDR3 COXs-2D2
LRQGSFDY

SEQ ID NO: 114 - protein, 8 aa, Artificial Sequence
  HCDR1 Beacon1-D10
GFTFSDHG

SEQ ID NO: 115 - protein, 8 aa, Artificial Sequence
  HCDR2 Beacon1-D10
INSGSSTI

SEQ ID NO: 116 - protein, 7 aa, Artificial Sequence
  HCDR3 Beacon1-D10
VRPGFAY

SEQ ID NO: 117 - protein, 8 aa, Artificial Sequence
  HCDR1 Beacon1-G12
GYTFTTYG

SEQ ID NO: 118 - protein, 8 aa, Artificial Sequence
  HCDR2 Beacon1-G12
INTYSGVP

SEQ ID NO: 119 - protein, 14 aa, Artificial Sequence
  HCDR3 Beacon1-G12
AIRRLSITLHGMDY

SEQ ID NO: 120 - protein, 8 aa, Artificial Sequence
  HCDR1 COXs-1B11
GFTFNTYA

SEQ ID NO: 121 - protein, 10 aa, Artificial Sequence
  HCDR2 COXs-1B11
VRSKVKNYAT

SEQ ID NO: 122 - protein, 8 aa, Artificial Sequence
  HCDR3 COXs-1B11
VRQGNFDV

SEQ ID NO: 123 - protein, 10 aa, Artificial Sequence
  HCDR2 COXs-2D4
IRSKSKNSAT

SEQ ID NO: 124 - protein, 8 aa, Artificial Sequence
  HCDR1 COXs-7A7
GFTFSDYG

SEQ ID NO: 125 - protein, 8 aa, Artificial Sequence
  HCDR3 COXs-9D6
LSQGSFDY

SEQ ID NO: 126 - protein, 10 aa, Artificial Sequence
  HCDR2 COXs-11H4
MRSESKNFAT

SEQ ID NO: 127 - protein, 10 aa, Artificial Sequence
  HCDR2 COXs-12E7
IRSKSKSYAT

SEQ ID NO: 128 - protein, 8 aa, Artificial Sequence
  HCDR1 CNE-7B1
GYTFTSSY

SEQ ID NO: 129 - protein, 8 aa, Artificial Sequence
  HCDR2 CNE-7B1
IYPGNDYT

SEQ ID NO: 130 - protein, 9 aa, Artificial Sequence
  HCDR3 CNE-7B1
TRSYYGFDY

SEQ ID NO: 131 - protein, 8 aa, Artificial Sequence
  HCDR1 CNF-2F8
GFSFQTYA

SEQ ID NO: 132 - protein, 10 aa, Artificial Sequence
  HCDR2 CNF-2F8
IRSKRNNYAT

SEQ ID NO: 133 - protein, 8 aa, Artificial Sequence
  HCDR1 CNGs-1C1
GFSFNAYA

SEQ ID NO: 134 - protein, 10 aa, Artificial Sequence
  HCDR2 CNGs-1C1
IRSKSNNYAT

SEQ ID NO: 135 - protein, 8 aa, Artificial Sequence
  HCDR1 CNGs-2F11
GYTFTEYY

SEQ ID NO: 136 - protein, 8 aa, Artificial Sequence
  HCDR2 CNGs-2F11
FNPYNGNT

SEQ ID NO: 137 - protein, 6 aa, Artificial Sequence
  HCDR3 CNGs-2F11
SRPYSN

SEQ ID NO: 138 - protein, 10 aa, Artificial Sequence
  HCDR2 CNE-6H7-G7
IRSKSNNYGT

SEQ ID NO: 139 - protein, 8 aa, Artificial Sequence
  HCDR3 CNE-6H7-G7
LSHGSSAY

SEQ ID NO: 140 - protein, 10 aa, Artificial Sequence
  HCDR2 CNGs-1B7-E4
IRSKSYNYAT

SEQ ID NO: 141 - protein, 6 aa, Artificial Sequence
  HCDR3 CNGs-1B7-E4
MRGQTY

SEQ ID NO: 142 - protein, 8 aa, Artificial Sequence
  HCDR1 CNGs-1E1-G5
GYTFTTSY

SEQ ID NO: 143 - protein, 8 aa, Artificial Sequence
  HCDR2 CNGs-1E1-G5
ISPGNVNT

SEQ ID NO: 144 - protein, 9 aa, Artificial Sequence
  HCDR3 CNGs-1E1-G5
AKSYFGFDF

SEQ ID NO: 145 - protein, 8 aa, Artificial Sequence
  HCDR1 CNGs-3H2-B7
GVSFKTYA

SEQ ID NO: 146 - protein, 10 aa, Artificial Sequence
  HCDR2 CNGs-3H2-B7
IRSKRYNYAT

SEQ ID NO: 147 - protein, 6 aa, Artificial Sequence
  HCDR3 CNGs-3H2-B7
MRQGAY

SEQ ID NO: 148 - protein, 10 aa, Artificial Sequence
  HCDR2 Beacon2-A3
IRTKRNNYVK

SEQ ID NO: 149 - protein, 6 aa, Artificial Sequence
  HCDR3 Beacon2-A3
VRQGAY

SEQ ID NO: 150 - protein, 8 aa, Artificial Sequence
  HCDR1 Beacon2-A7
GFTFSNYW

SEQ ID NO: 151 - protein, 10 aa, Artificial Sequence
  HCDR2 Beacon2-A7
IRLKSDNYAT

SEQ ID NO: 152 - protein, 8 aa, Artificial Sequence
  HCDR3 Beacon2-A7
APYSNWGY

SEQ ID NO: 153 - protein, 6 aa, Artificial Sequence
  HCDR3 Beacon2-E5
VRQGAF

SEQ ID NO: 154 - protein, 10 aa, Artificial Sequence
  HCDR2 Genovac1-D12
IRLKSDNYGT

SEQ ID NO: 155 - protein, 8 aa, Artificial Sequence
  HCDR3 Genovac1-D12
TSNAWFAY

SEQ ID NO: 156 - protein, 8 aa, Artificial Sequence
  HCDR2 Beacon1-A4
IKPYSGRT

SEQ ID NO: 157 - protein, 21 aa, Artificial Sequence
  HCDR3 Beacon1-A4
AREGVSITTVGSSLYYYPMDY

SEQ ID NO: 158 - protein, 6 aa, Artificial Sequence
  LCDR1 Genovac2-C1
ENIYSY

SEQ ID NO: 159 - protein, 3 aa, Artificial Sequence
  LCDR2 Genovac2-C1
NAK

SEQ ID NO: 160 - protein, 9 aa, Artificial Sequence
  LCDR3 Genovac2-C1
QHHYGIPRT

SEQ ID NO: 161 - protein, 9 aa, Artificial Sequence
  LCDR3 Genovac1-G2
QHHYGTPWT

SEQ ID NO: 162 - protein, 6 aa, Artificial Sequence
  LCDR1 Beacon2-G5
QDVSTA

SEQ ID NO: 163 - protein, 3 aa, Artificial Sequence
  LCDR2 Beacon2-G5
SAS

SEQ ID NO: 164 - protein, 9 aa, Artificial Sequence
  LCDR3 Beacon2-G5
QQHYTTPYT

SEQ ID NO: 165 - protein, 6 aa, Artificial Sequence
  LCDR1 Beacon2-F1
QNVGTA

SEQ ID NO: 166 - protein, 9 aa, Artificial Sequence
  LCDR3 Beacon2-F1
QQYSSYPYT

SEQ ID NO: 167 - protein, 11 aa, Artificial Sequence
  LCDR1 CNE-6E5-C1
QTIVHSNGVTY

SEQ ID NO: 168 - protein, 3 aa, Artificial Sequence
  LCDR2 CNE-6E5-C1
KVS

SEQ ID NO: 169 - protein, 9 aa, Artificial Sequence
  LCDR3 CNE-6E5-C1
FQGSHVPPT

SEQ ID NO: 170 - protein, 6 aa, Artificial Sequence
  LCDR1 CNGs-3B2
KSISKY

SEQ ID NO: 171 - protein, 3 aa, Artificial Sequence
  LCDR2 CNGs-3B2
SGS

SEQ ID NO: 172 - protein, 9 aa, Artificial Sequence
  LCDR2 CNGs-3B2
QQHNEFPWT

SEQ ID NO: 173 - protein, 10 aa, Artificial Sequence
  LCDR1 Beacon1-B2
ESVSVRGTGL

SEQ ID NO: 174 - protein, 3 aa, Artificial Sequence
  LCDR2 Beacon1-B2
AAS

SEQ ID NO: 175 - protein, 8 aa, Artificial Sequence
  LCDR3 Beacon1-B2
QQSREYRT

SEQ ID NO: 176 - protein, 11 aa, Artificial Sequence
  LCDR1 CNGs-1B7
KSLLHSNGNTY

SEQ ID NO: 177 - protein, 3 aa, Artificial Sequence
  LCDR2 CNGs-1B7
SVS

SEQ ID NO: 178 - protein, 9 aa, Artificial Sequence
  LCDR3 CNGs-1B7
MQSLEYPYT

SEQ ID NO: 179 - protein, 11 aa, Artificial Sequence
  LCDR1 CNE-6E10
QSLLDINGKTY

SEQ ID NO: 180 - protein, 3 aa, Artificial Sequence
  LCDR2 CNE-6E10
LVS

SEQ ID NO: 181 - protein, 9 aa, Artificial Sequence
  LCDR3 CNE-6E10
WQGTHFPQT

SEQ ID NO: 182 - protein, 12 aa, Artificial Sequence
  LCDR1 COXs-1B10
QSLLSSRTQKNY

SEQ ID NO: 183 - protein, 3 aa, Artificial Sequence
  LCDR2 COXs-1B10
FAS

SEQ ID NO: 184 - protein, 9 aa, Artificial Sequence
  LCDR3 COXs-1B10
QQYFLTPYT

SEQ ID NO: 185 - protein, 11 aa, Artificial Sequence
  LCDR1 COXs-2D2
QRIVHTDGNTY

SEQ ID NO: 186 - protein, 11 aa, Artificial Sequence
  LCDR2 COXs-2D2
QRIVHTDGNTY

SEQ ID NO: 187 - protein, 9 aa, Artificial Sequence
  LCDR3 COXs-2D2
FQGTHFPYT

SEQ ID NO: 188 - protein, 11 aa, Artificial Sequence
  LCDR1 Beacon1-D10
RTLCTMMETRI

SEQ ID NO: 189 - protein, 9 aa, Artificial Sequence
  LCDR3 Beacon1-D10
FQATHDPFT

SEQ ID NO: 190 - protein, 6 aa, Artificial Sequence
  LCDR1 Beacon1-G12
ENIYSN

SEQ ID NO: 191 - protein, 3 aa, Artificial Sequence
  LCDR2 Beacon1-G12
GAT

SEQ ID NO: 192 - protein, 9 aa, Artificial Sequence
  LCDR3 Beacon1-G12
QHFWGTPWT

SEQ ID NO: 193 - protein, 11 aa, Artificial Sequence
  LCDR1 COXs-1B11
QNIVHTDGNTY

SEQ ID NO: 194 - protein, 3 aa, Artificial Sequence
  LCDR2 COXs-1B11
KIS

SEQ ID NO: 195 - protein, 9 aa, Artificial Sequence
  LCDR3 COXs-1B11
FQLTHFPYT

SEQ ID NO: 196 - protein, 11 aa, Artificial Sequence
  LCDR1 COXs-4H5
QRIVHSDGNTY

SEQ ID NO: 197 - protein, 11 aa, Artificial Sequence
  LCDR1 COXs-7A7
QSLLHRNGNTY

SEQ ID NO: 198 - protein, 3 aa, Artificial Sequence
  LCDR2 COXs-7A7
EIF

SEQ ID NO: 199 - protein, 9 aa, Artificial Sequence
  LCDR3 COXs-7A7
MQATYEPLT

SEQ ID NO: 200 - protein, 9 aa, Artificial Sequence
  LCDR3 COXs-11H4
FQLTHVPYT

SEQ ID NO: 201 - protein, 11 aa, Artificial Sequence
  LCDR1 CNE-7B1
QSIVRSRGNTY

SEQ ID NO: 202 - protein, 9 aa, Artificial Sequence
  LCDR3 CNE-7B1
FQGSHVPYT

SEQ ID NO: 203 - protein, 11 aa, Artificial Sequence
  LCDR1 CNF-2F8
QSLLDSDGKTY

SEQ ID NO: 204 - protein, 11 aa, Artificial Sequence
  LCDR1 CNGs-1C1
QSLLYSDGKTY

SEQ ID NO: 205 - protein, 9 aa, Artificial Sequence
  LCDR3 CNGs-1C1
WQATHFPQT

SEQ ID NO: 206 - protein, 11 aa, Artificial Sequence
  LCDR1 CNGs-2F11
QSIVHSNGNTY

SEQ ID NO: 207 - protein, 9 aa, Artificial Sequence
  LCDR3 CNGs-2F11
FQGSHVPFT

SEQ ID NO: 208 - protein, 11 aa, Artificial Sequence
  LCDR1 CNE-6H7-G7
QSLLYSDGNTY

SEQ ID NO: 209 - protein, 11 aa, Artificial Sequence
  LCDR1 CNGs-1B7-E4
KSLLNSDGFTY

SEQ ID NO: 210 - protein, 9 aa, Artificial Sequence
  LCDR3 CNGs-1B7-E4
FQSNYLPFT

SEQ ID NO: 211 - protein, 11 aa, Artificial Sequence
  LCDR1 CNGs-3G6-G5
QSLVHSNGNTF

SEQ ID NO: 212 - protein, 9 aa, Artificial Sequence
  LCDR3 Beacon2-A3
FQSNYLPYT

SEQ ID NO: 213 - protein, 11 aa, Artificial Sequence
  LCDR1 Beacon2-A7
KSLLHTNGNTY

SEQ ID NO: 214 - protein, 3 aa, Artificial Sequence
  LCDR2 Beacon2-A7
RMS

SEQ ID NO: 215 - protein, 9 aa, Artificial Sequence
  LCDR3 Beacon2-A7
MQYLEYPYT

SEQ ID NO: 216 - protein, 3 aa, Artificial Sequence
  LCDR2 Genovac1-D12
YMS

SEQ ID NO: 217 - protein, 9 aa, Artificial Sequence
  LCDR3 Genovac1-D12
LLSLEYPYT

SEQ ID NO: 218 - protein, 6 aa, Artificial Sequence
  LCDR1 Genovac2-H2
ENIYNN

SEQ ID NO: 219 - protein, 3 aa, Artificial Sequence
  LCDR2 Genovac2-H2
AAT

SEQ ID NO: 220 - protein, 6 aa, Artificial Sequence
  LCDR1 Beacon1-A4
QSISNN

SEQ ID NO: 221 - protein, 3 aa, Artificial Sequence
  LCDR2 Beacon1-A4
YAS

SEQ ID NO: 222 - protein, 9 aa, Artificial Sequence
  LCDR3 Beacon1-A4
QQSNSWPRT

SEQ ID NO: 223 - protein, 10 aa, Artificial Sequence
  HCDR1 Genovac2-C1
GFTFSSYTMS

SEQ ID NO: 224 - protein, 17 aa, Artificial Sequence
  HCDR2 Genovac2-C1
TISSGGSYTYYPDSVKG

SEQ ID NO: 225 - protein, 12 aa, Artificial Sequence
  HCDR3 Genovac2-C1
GGGNYPFWFFDV

SEQ ID NO: 226 - protein, 16 aa, Artificial Sequence
  HCDR2 Genovac1-G2
YIRYDGSNNYNPSLKN

SEQ ID NO: 227 - protein, 9 aa, Artificial Sequence
  HCDR3 Genovac1-G2
DGYSTYFDY

SEQ ID NO: 228 - protein, 10 aa, Artificial Sequence
  HCDR1 Beacon2-G5
GFTFSSYGMS

SEQ ID NO: 229 - protein, 17 aa, Artificial Sequence
  HCDR2 Beacon2-G5
TISSRGSYTYYPDSVKG

SEQ ID NO: 230 - protein, 10 aa, Artificial Sequence
  HCDR3 Beacon2-G5
RVFPYWFFDV

SEQ ID NO: 231 - protein, 12 aa, Artificial Sequence
  HCDR3 Beacon2-F1
LYGNYPYFAMDY

SEQ ID NO: 232 - protein, 12 aa, Artificial Sequence
  HCDR1 CNE-6E5-C1
GFSLITSGMGVS

SEQ ID NO: 233 - protein, 16 aa, Artificial Sequence
  HCDR2 CNE-6E5-C1
HIYWDDEKRYNPSLKS

SEQ ID NO: 234 - protein, 10 aa, Artificial Sequence
  HCDR3 CNE-6E5-C1
REDFGDGFDY

SEQ ID NO: 235 - protein, 10 aa, Artificial Sequence
  HCDR1 CNGs-3B2
GFTFSSFGMH

SEQ ID NO: 236 - protein, 17 aa, Artificial Sequence
  HCDR2 CNGs-3B2
YISSGSTTIYYADTVKG

SEQ ID NO: 237 - protein, 10 aa, Artificial Sequence
  HCDR3 CNGs-3B2
SHWPYWYFDV

SEQ ID NO: 238 - protein, 10 aa, Artificial Sequence
  HCDR1 Beacon1-B2
GYTFTDYYMN

SEQ ID NO: 239 - protein, 17 aa, Artificial Sequence
  HCDR2 Beacon1-B2
DINPNNGGTTYNQKFKG

SEQ ID NO: 240 - protein, 11 aa, Artificial Sequence
  HCDR3 Beacon1-B2
SRRNWGYAMDY

SEQ ID NO: 241 - protein, 10 aa, Artificial Sequence
  HCDR1 CNGs-1B7
GFTFSDYWMN

SEQ ID NO: 242 - protein, 19 aa, Artificial Sequence
  HCDR2 CNGs-1B7
QIRLNSGNYAARYAESVKG

SEQ ID NO: 243 - protein, 6 aa, Artificial Sequence
  HCDR3 CNGs-1B7
NAWLAN

SEQ ID NO: 244 - protein, 10 aa, Artificial Sequence
  HCDR1 CNE-6E10
GFSFNTYAMN

SEQ ID NO: 245 - protein, 19 aa, Artificial Sequence
  HCDR2 CNE-6E10
RIRSKTNSYETYYADSVKD

SEQ ID NO: 246 - protein, 6 aa, Artificial Sequence
  HCDR3 CNE-6E10
HGAFAY

SEQ ID NO: 247 - protein, 12 aa, Artificial Sequence
  HCDR1 COXs-1B10
GFSLSTSGMGIG

SEQ ID NO: 248 - protein, 16 aa, Artificial Sequence
  HCDR2 COXs-1B10
HIWWDDEKYYRPSLKS

SEQ ID NO: 249 - protein, 8 aa, Artificial Sequence
  HCDR3 COXs-1B10
RTPGRFAY

SEQ ID NO: 250 - protein, 10 aa, Artificial Sequence
  HCDR1 COXs-2D2
GFSFDTYAMN

SEQ ID NO: 251 - protein, 19 aa, Artificial Sequence
  HCDR2 COXs-2D2
RIRSKSKNYATYYADSVKD

SEQ ID NO: 252 - protein, 6 aa, Artificial Sequence
  HCDR3 COXs-2D2
QGSFDY

SEQ ID NO: 253 - protein, 10 aa, Artificial Sequence
  HCDR1 Beacon1-D10
GFTFSDHGMH

SEQ ID NO: 254 - protein, 17 aa, Artificial Sequence
  HCDR2 Beacon1-D10
YINSGSSTIHYADSVKG

SEQ ID NO: 255 - protein, 5 aa, Artificial Sequence
  HCDR3 Beacon1-D10
PGFAY

SEQ ID NO: 256 - protein, 10 aa, Artificial Sequence
  HCDR1 Beacon1-G12
GYTFTTYGMS

SEQ ID NO: 257 - protein, 17 aa, Artificial Sequence
  HCDR2 Beacon1-G12
WINTYSGVPTYADDFKG

SEQ ID NO: 258 - protein, 12 aa, Artificial Sequence
  HCDR3 Beacon1-G12
RRLSITLHGMDY

SEQ ID NO: 259 - protein, 10 aa, Artificial Sequence
  HCDR1 COXs-1B11
GFTFNTYAMN

SEQ ID NO: 260 - protein, 19 aa, Artificial Sequence
  HCDR2 COXs-1B11
RVRSKVKNYATYYADSVKD

SEQ ID NO: 261 - protein, 6 aa, Artificial Sequence
  HCDR3 COXs-1B11
QGNFDV

SEQ ID NO: 262 - protein, 19 aa, Artificial Sequence
  HCDR2 COXs-2D4
RIRSKSKNSATYYADSVKD

SEQ ID NO: 263 - protein, 10 aa, Artificial Sequence
  HCDR1 COXs-7A7
GFTFSDYGMH

SEQ ID NO: 264 - protein, 19 aa, Artificial Sequence
  HCDR2 COXs-9D6
RIRSKSKNYATYYADSVTD

SEQ ID NO: 265 - protein, 19 aa, Artificial Sequence
  HCDR2 COXs-11H4
RMRSESKNFATYYADSVKD

SEQ ID NO: 266 - protein, 19 aa, Artificial Sequence
  HCDR2 COXs-12E7
RIRSKSKSYATYYADSVKD

SEQ ID NO: 267 - protein, 10 aa, Artificial Sequence
  HCDR1 CNE-7B1
GYTFTSSYVH

SEQ ID NO: 268 - protein, 17 aa, Artificial Sequence
  HCDR2 CNE-7B1
WIYPGNDYTKYNEKFKG

SEQ ID NO: 269 - protein, 7 aa, Artificial Sequence
  HCDR3 CNE-7B1
SYYGFDY

SEQ ID NO: 270 - protein, 10 aa, Artificial Sequence
  HCDR1 CNF-2F8
GFSFQTYAMN

SEQ ID NO: 271 - protein, 19 aa, Artificial Sequence
  HCDR2 CNF-2F8
RIRSKRNNYATYYADSVKD

SEQ ID NO: 272 - protein, 10 aa, Artificial Sequence
  HCDR1 CNGs-1C1
GFSFNAYAMN

SEQ ID NO: 273 - protein, 19 aa, Artificial Sequence
  HCDR2 CNGs-1C1
RIRSKSNNYATYYADSVKD

SEQ ID NO: 274 - protein, 10 aa, Artificial Sequence
  HCDR1 CNGs-2F11
GYTFTEYYIN

SEQ ID NO: 275 - protein, 17 aa, Artificial Sequence
  HCDR2 CNGs-2F11
IFNPYNGNTKYNQNFKG

SEQ ID NO: 276 - protein, 4 aa, Artificial Sequence
  HCDR3 CNGs-2F11
PYSN

SEQ ID NO: 277 - protein, 19 aa, Artificial Sequence
  HCDR2 CNE-6H7-G7
RIRSKSNNYGTYYADSVKD

SEQ ID NO: 278 - protein, 6 aa, Artificial Sequence
  HCDR3 CNE-6H7-G7
HGSSAY

SEQ ID NO: 279 - protein, 19 aa, Artificial Sequence
  HCDR2 CNGs-1B7-E4
RIRSKSYNYATYHAGSVKD

SEQ ID NO: 280 - protein, 4 aa, Artificial Sequence
  HCDR3 CNGs-1B7-E4
GQTY

SEQ ID NO: 281 - protein, 10 aa, Artificial Sequence
  HCDR1 CNGs-1E1-G5
GYTFTTSYIH

SEQ ID NO: 282 - protein, 17 aa, Artificial Sequence
  HCDR2 CNGs-1E1-G5
WISPGNVNTRSNEKFKG

SEQ ID NO: 283 - protein, 7 aa, Artificial Sequence
  HCDR3 CNGs-1E1-G5
SYFGFDF

SEQ ID NO: 284 - protein, 17 aa, Artificial Sequence
  HCDR2 CNGs-3G6-G5
IFNPYNGNTKYNQKFKG

SEQ ID NO: 285 - protein, 10 aa, Artificial Sequence
  HCDR1 CNGs-3H2-B
GVSFKTYAMT

SEQ ID NO: 286 - protein, 19 aa, Artificial Sequence
  HCDR2 CNGs-3H2-B7
RIRSKRYNYATYYADSVKD

SEQ ID NO: 287 - protein, 4 aa, Artificial Sequence
  HCDR3 CNGs-3H2-B7
QGAY

SEQ ID NO: 288 - protein, 19 aa, Artificial Sequence
  HCDR2 Beacon2-A3
RIRTKRNNYVKFYADSVKD

SEQ ID NO: 289 - protein, 10 aa, Artificial Sequence
  HCDR1Beacon2-A7
GFTFSNYWMS

SEQ ID NO: 290 - protein, 19 aa, Artificial Sequence
  HCDR2 Beacon2-A7
QIRLKSDNYATHYAESVKG

SEQ ID NO: 291 - protein, 6 aa, Artificial Sequence
  HCDR3 Beacon2-A7
YSNWGY

SEQ ID NO: 292 - protein, 19 aa, Artificial Sequence
  HCDR2 Beacon2-E5
RIRTKRNNYVKFYADSVKG

SEQ ID NO: 293 - protein, 4 aa, Artificial Sequence
  HCDR3 Beacon2-E5
QGAF

SEQ ID NO: 294 - protein, 10 aa, Artificial Sequence
  HCDR1 Genovac1-D12
GFTFSNYWMN

SEQ ID NO: 295 - protein, 19 aa, Artificial Sequence
  HCDR2 Genovac1-D12
QIRLKSDNYGTHYAESVKG

SEQ ID NO: 296 - protein, 6 aa, Artificial Sequence
  HCDR3 Genovac1-D12
NAWFAY

SEQ ID NO: 297 - protein, 17 aa, Artificial Sequence
  HCDR2 Beacon1-A4
VIKPYSGRTSYDQKFKD

SEQ ID NO: 298 - protein, 19 aa, Artificial Sequence
  HCDR3 Beacon1-A4
EGVSITTVGSSLYYYPMDY

SEQ ID NO: 299 - protein, 11 aa, Artificial Sequence
  LCDR1 Genovac2-C1
RASENIYSYLA

SEQ ID NO: 300 - protein, 7 aa, Artificial Sequence
  LCDR2 Genovac2-C1
NAKTLAE

SEQ ID NO: 301 - protein, 11 aa, Artificial Sequence
  LCDR1 Beacon2-G5
KASQDVSTAVA

SEQ ID NO: 302 - protein, 7 aa, Artificial Sequence
  LCDR2 Beacon2-G5
SASYRYT

SEQ ID NO: 303 - protein, 11 aa, Artificial Sequence
  LCDR1 Beacon2-F1
KASQNVGTAVA

SEQ ID NO: 304 - protein, 7 aa, Artificial Sequence
  LCDR2 Beacon2-F1
SASNRYT

SEQ ID NO: 305 - protein, 16 aa, Artificial Sequence
  LCDR1 CNE-6E5-C1
RSSQTIVHSNGVTYLE

SEQ ID NO: 306 - protein, 7 aa, Artificial Sequence
  LCDR2 CNE-6E5-C1
KVSNRFS

SEQ ID NO: 307 - protein, 11 aa, Artificial Sequence
  LCDR1 CNGs-3B2
RASKSISKYLA

SEQ ID NO: 308 - protein, 7 aa, Artificial Sequence
  LCDR2 CNGs-3B2
SGSTLQS

SEQ ID NO: 309 - protein, 15 aa, Artificial Sequence
  LCDR1 Beacon1-B2
RASESVSVRGTGLMH

SEQ ID NO: 310 - protein, 7 aa, Artificial Sequence
  LCDR2 Beacon1-B2
AASNLES

SEQ ID NO: 311 - protein, 16 aa, Artificial Sequence
  LCDR1 CNGs-1B7
RSSKSLLHSNGNTYLY

SEQ ID NO: 312 - protein, 7 aa, Artificial Sequence
  LCDR2 CNGs-1B7
SVSNLAS

SEQ ID NO: 313 - protein, 16 aa, Artificial Sequence
  LCDR1 CNE-6E10
KSSQSLLDINGKTYLN

SEQ ID NO: 314 - protein, 7 aa, Artificial Sequence
  LCDR2 CNE-6E10
LVSKLDS

SEQ ID NO: 315 - protein, 17 aa, Artificial Sequence
  LCDR1 COXs-1B10
KSSQSLLSSRTQKNYLA

SEQ ID NO: 316 - protein, 7 aa, Artificial Sequence
  LCDR2 COXs-1B10
FASIRES

SEQ ID NO: 317 - protein, 16 aa, Artificial Sequence
  LCDR1 COXs-2D2
RSGQRIVHTDGNTYLY

SEQ ID NO: 318 - protein, 7 aa, Artificial Sequence
  LCDR2 COXs-2D2
RVSNRFS

SEQ ID NO: 319 - protein, 11 aa, Artificial Sequence
  LCDR1 Beacon1-G12
RASENIYSNLA

SEQ ID NO: 320 - protein, 7 aa, Artificial Sequence
  LCDR2 Beacon1-G12
GATNLAD

SEQ ID NO: 321 - protein, 16 aa, Artificial Sequence
  LCDR1 COXs-1B11
RSGQNIVHTDGNTYLY

SEQ ID NO: 322 - protein, 7 aa, Artificial Sequence
  LCDR2 COXs-1B11
KISNRFS

SEQ ID NO: 323 - protein, 16 aa, Artificial Sequence
  LCDR1 COXs-2D4
RSSQRIVHTDGNTYLY

SEQ ID NO: 324 - protein, 16 aa, Artificial Sequence
  LCDR1 COXs-4H5
RSSQRIVHSDGNTYLY

SEQ ID NO: 325 - protein, 16 aa, Artificial Sequence
  LCDR1 COXs-7A7
RSSQSLLHRNGNTYLN

SEQ ID NO: 326 - protein, 7 aa, Artificial Sequence
  LCDR2 COXs-7A7
EIFERKS

SEQ ID NO: 327 - protein, 16 aa, Artificial Sequence
  LCDR1 CNE-7B1
RSGQSIVRSRGNTYLE

SEQ ID NO: 328 - protein, 16 aa, Artificial Sequence
  LCDR1 CNF-2F8
KSSQSLLDSDGKTYLN

SEQ ID NO: 329 - protein, 16 aa, Artificial Sequence
  LCDR1 CNGs-1C1
KSSQSLLYSDGKTYLN

SEQ ID NO: 330 - protein, 16 aa, Artificial Sequence
  LCDR1 CNGs-2F11
RSSQSIVHSNGNTYLE

SEQ ID NO: 331 - protein, 16 aa, Artificial Sequence
  LCDR1 CNE-6H7-G7
RSSQSLLYSDGNTYLN

SEQ ID NO: 332 - protein, 7 aa, Artificial Sequence
  LCDR2 CNE-6H7-G7
LVSELDS

SEQ ID NO: 333 - protein, 16 aa, Artificial Sequence
  LCDR1 CNGs-1B7-E4
KSTKSLLNSDGFTYLD

SEQ ID NO: 334 - protein, 7 aa, Artificial Sequence
  LCDR2 CNGs-1B7-E4
LVSNRFS

SEQ ID NO: 335 - protein, 7 aa, Artificial Sequence
  LCDR2 CNGs-1E1-G5
KVSNRSS

SEQ ID NO: 336 - protein, 16 aa, Artificial Sequence
  LCDR1 CNGs-3G6-G5
RSSQSLVHSNGNTFLE

SEQ ID NO: 337 - protein, 16 aa, Artificial Sequence
  LCDR1 Beacon2-A7
RSTKSLLHTNGNTYLY

SEQ ID NO: 338 - protein, 7 aa, Artificial Sequence
  LCDR2 Beacon2-A7
RMSNLAS

SEQ ID NO: 339 - protein, 16 aa, Artificial Sequence
  LCDR1 Genovac1-D12
RSSKSLLHSNGNTYLF

SEQ ID NO: 340 - protein, 7 aa, Artificial Sequence
  LCDR2 Genovac1-D12
YMSNLAS

SEQ ID NO: 341 - protein, 11 aa, Artificial Sequence
  LCDR1 Genovac2-H2
RASENIYNNLA

SEQ ID NO: 342 - protein, 7 aa, Artificial Sequence
  LCDR2 Genovac2-H2
AATNLAD

SEQ ID NO: 343 - protein, 11 aa, Artificial Sequence
  LCDR1 Beacon1-A4
RASQSISNNLH

SEQ ID NO: 344 - protein, 7 aa, Artificial Sequence
  LCDR2 Beacon1-A4
YASQSVS

SEQ ID NO: 345 - protein, 330 aa, Homo sapiens [sequence omitted]

SEQ ID NO: 346 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 107 aa, Artificial Sequence [sequence omitted]

SEQ ID NO: 348 - protein, 106 aa, Artificial Sequence
  antibody light chain constant region sequence
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 349 - protein, 120 aa, Artificial Sequence [sequence omitted]

SEQ ID NO: 350 - protein, 111 aa, Artificial Sequence [sequence omitted]

SEQ ID NO: 351 - protein, 113 aa, Artificial Sequence [sequence omitted]

SEQ ID NO: 352 - protein, 107 aa, Artificial Sequence [sequence omitted]

SEQ ID NO: 353 - protein, 123 aa, Artificial Sequence
  VH and VL Amino Acid Sequences of Checkpoint Inhibitor
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 354 - protein, 107 aa, Artificial Sequence
  VH and VL Amino Acid Sequences of Checkpoint Inhibitor
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 355 - protein, 116 aa, Artificial Sequence
  VH and VL Amino Acid Sequences of Checkpoint Inhibitor
VQLVESGGGLVQPGGSLRLSCAASGFAFSRYDMSWVRQAPGKGLESVAYISGGGANTYYLDNVKGRFTISRDNAKNSLYLQMNSLRAEDTAVYYCASPYLSYFDVWGQGTLVTVSS

SEQ ID NO: 356 - protein, 107 aa, Artificial Sequence
  VH and VL Amino Acid Sequences of Checkpoint Inhibitor
EIVMTQSPATLSVSPGERATLSCRASQSLSDYLHWYQQKPGQAPRLLIKSASQSISGIPARFSGSGSGTEFTLTISSLQSEDFAVYYCQNGHSFPYTFGQGTKLEIK

SEQ ID NO: 357 - protein, 121 aa, Artificial Sequence
  VH and VL Amino Acid Sequences of Checkpoint Inhibitor
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 358 - protein, 108 aa, Artificial Sequence
  VH and VL Amino Acid Sequences of Checkpoint Inhibitor
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 359 - protein, 118 aa, Artificial Sequence [sequence omitted]

SEQ ID NO: 360 - protein, 107 aa, Artificial Sequence [sequence omitted]

SEQ ID NO: 361 - protein, 120 aa, Artificial Sequence
  VH and VL Amino Acid Sequences of Checkpoint Inhibitor
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 362 - protein, 110 aa, Artificial Sequence
  VH and VL Amino Acid Sequences of Checkpoint Inhibitor
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 363 - protein, 117 aa, Artificial Sequence
  VH and VL Amino Acid Sequences of Checkpoint Inhibitor
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSS

SEQ ID NO: 364 - protein, 107 aa, Artificial Sequence
  VH and VL Amino Acid Sequences of Checkpoint Inhibitor
EIVLTQSPATLSLSPGERATLSCRASQSVNDYLAWYQQKPGQAPRLLIYDASNRATGIPARFSGSGSGTDFTLTISSLEPEDFAVYYCQQGGHAPITFGQGTKVEIK

SEQ ID NO: 365 - protein, 124 aa, Artificial Sequence
  VH and VL Amino Acid Sequences of Checkpoint Inhibitor
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSS

SEQ ID NO: 366 - protein, 106 aa, Artificial Sequence
  VH and VL Amino Acid Sequences of Checkpoint Inhibitor
DIQMTQSPSSLSASVGDRVTITCKASENVGTFVSWYQQKPGKAPKLLIYGASNRYTGVPSRFSGSGSGTDFTLTISSLQPEDFATYYCGQSYSYPTFGQGTKLEIK